SEQUENCE LISTING (83 sequences)

SEQ ID NO: 1 - protein, 97 aa, Synthetic construct
  source 1..97: note = Neo-2/15 (without 3 N-terminal amino acids; -3 AA )
KIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 2 - protein, 100 aa, Synthetic construct
  source 1..100: note = Neo-2/15
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 3 - protein, 97 aa, Synthetic construct
  source 1..97: note = Neo_2/15_ D15S_Q95E (-3 AA)
KIQLHAEHALYSALMILNIVKTNSPPAEEKLEDYAFNFELILEEIARLFESGDQKDEAEKAKRMKEWMKRIKTTASEDEQEEMANAIITILESWIFS

SEQ ID NO: 4 - protein, 100 aa, Synthetic construct
  source 1..100: note = Neo_2/15_D15S_Q95E
PKKKIQLHAEHALYSALMILNIVKTNSPPAEEKLEDYAFNFELILEEIARLFESGDQKDEAEKAKRMKEWMKRIKTTASEDEQEEMANAIITILESWIFS

SEQ ID NO: 5 - protein, 100 aa, Synthetic construct
  source 1..100: note = Neo_2/15_D15S_Q95E_H11F
PKKKIQLHAEFALYSALMILNIVKTNSPPAEEKLEDYAFNFELILEEIARLFESGDQKDEAEKAKRMKEWMKRIKTTASEDEQEEMANAIITILESWIFS

SEQ ID NO: 6 - protein, 97 aa, Synthetic construct
  source 1..97: note = Neo_2/15_Q95E_N40S_I44S (-3AA)
KIQLHAEHALYDALMILNIVKTNSPPAEEKLEDYAFSFELSLEEIARLFESGDQKDEAEKAKRMKEWMKRIKTTASEDEQEEMANAIITILESWIFS

SEQ ID NO: 7 - protein, 100 aa, Synthetic construct
  source 1..100: note = Neo_2/15_Q95E_N40S_I44S
PKKKIQLHAEHALYDALMILNIVKTNSPPAEEKLEDYAFSFELSLEEIARLFESGDQKDEAEKAKRMKEWMKRIKTTASEDEQEEMANAIITILESWIFS

SEQ ID NO: 8 - protein, 100 aa, Synthetic construct
  source 1..100: note = Neo_2/15_Q95E_N40A_I44A
PKKKIQLHAEHALYDALMILNIVKTNSPPAEEKLEDYAFAFELALEEIARLFESGDQKDEAEKAKRMKEWMKRIKTTASEDEQEEMANAIITILESWIFS

SEQ ID NO: 9 - protein, 100 aa, Synthetic construct
  source 1..100: note = Neo_2/15_Q95E_N40S_I44A
PKKKIQLHAEHALYDALMILNIVKTNSPPAEEKLEDYAFSFELALEEIARLFESGDQKDEAEKAKRMKEWMKRIKTTASEDEQEEMANAIITILESWIFS

SEQ ID NO: 10 - protein, 100 aa, Synthetic construct
  source 1..100: note = Neo_2/15_Q95E_N40A_I44S
PKKKIQLHAEHALYDALMILNIVKTNSPPAEEKLEDYAFAFELSLEEIARLFESGDQKDEAEKAKRMKEWMKRIKTTASEDEQEEMANAIITILESWIFS

SEQ ID NO: 11 - protein, 100 aa, Synthetic construct
  source 1..100: note = Neo_2/15_ Q95E_N40S_I44S_H11F
PKKKIQLHAEFALYDALMILNIVKTNSPPAEEKLEDYAFSFELSLEEIARLFESGDQKDEAEKAKRMKEWMKRIKTTASEDEQEEMANAIITILESWIFS

SEQ ID NO: 12 - protein, 100 aa, Synthetic construct
  source 1..100: note = Neo_2/15_ Q95E_N40S_I44S_Y14K
PKKKIQLHAEHALKDALMILNIVKTNSPPAEEKLEDYAFSFELSLEEIARLFESGDQKDEAEKAKRMKEWMKRIKTTASEDEQEEMANAIITILESWIFS

SEQ ID NO: 13 - protein, 100 aa, Synthetic construct
  source 1..100: note = Neo_2/15_D15S_Q95E_Y14K
PKKKIQLHAEHALKSALMILNIVKTNSPPAEEKLEDYAFNFELILEEIARLFESGDQKDEAEKAKRMKEWMKRIKTTASEDEQEEMANAIITILESWIFS

SEQ ID NO: 14 - protein, 97 aa, Synthetic construct
  source 1..97: note = Neo_2/15_Q95H_D15A (-3 AA)
KIQLHAEHALYAALMILNIVKTNSPPAEEKLEDYAFNFELILEEIARLFESGDQKDEAEKAKRMKEWMKRIKTTASEDEQEEMANAIITILHSWIFS

SEQ ID NO: 15 - protein, 100 aa, Synthetic construct
  source 1..100: note = Neo_2/15_Q95H_D15A
PKKKIQLHAEHALYAALMILNIVKTNSPPAEEKLEDYAFNFELILEEIARLFESGDQKDEAEKAKRMKEWMKRIKTTASEDEQEEMANAIITILHSWIFS

SEQ ID NO: 16 - protein, 97 aa, Synthetic construct
  source 1..97: note = Neo_2/15_D15A_Q95A (-3 AA)
KIQLHAEHALYAALMILNIVKTNSPPAEEKLEDYAFNFELILEEIARLFESGDQKDEAEKAKRMKEWMKRIKTTASEDEQEEMANAIITILASWIFS

SEQ ID NO: 17 - protein, 100 aa, Synthetic construct
  source 1..100: note = Neo_2/15_D15A_Q95A
PKKKIQLHAEHALYAALMILNIVKTNSPPAEEKLEDYAFNFELILEEIARLFESGDQKDEAEKAKRMKEWMKRIKTTASEDEQEEMANAIITILASWIFS

SEQ ID NO: 18 - protein, 100 aa, Synthetic construct
  source 1..100: note = Neo_2/15_D15S_Y14K
PKKKIQLHAEHALKSALMILNIVKTNSPPAEEKLEDYAFNFELILEEIARLFESGDQKDEAEKAKRMKEWMKRIKTTASEDEQEEMANAIITILQSWIFS

SEQ ID NO: 19 - protein, 100 aa, Synthetic construct
  source 1..100: note = Neo_2/15_ N40S_I44S_H11F
PKKKIQLHAEFALYDALMILNIVKTNSPPAEEKLEDYAFSFELSLEEIARLFESGDQKDEAEKAKRMKEWMKRIKTTASEDEQEEMANAIITILQSWIFS

SEQ ID NO: 20 - protein, 97 aa, Synthetic construct
  source 1..97: note = Neo_2/15_ N40S_I44S_H11F (-3 AA)
KIQLHAEFALYDALMILNIVKTNSPPAEEKLEDYAFSFELSLEEIARLFESGDQKDEAEKAKRMKEWMKRIKTTASEDEQEEMANAIITILQSWIFS

SEQ ID NO: 21 - protein, 100 aa, Synthetic construct
  source 1..100: note = Neo_2/15_ N40S_I44S_D15S
PKKKIQLHAEHALYSALMILNIVKTNSPPAEEKLEDYAFSFELSLEEIARLFESGDQKDEAEKAKRMKEWMKRIKTTASEDEQEEMANAIITILQSWIFS

SEQ ID NO: 22 - protein, 100 aa, Synthetic construct
  source 1..100: note = Neo_2/15_ N40S_I44S_Y14K_D15S
PKKKIQLHAEHALKSALMILNIVKTNSPPAEEKLEDYAFSFELSLEEIARLFESGDQKDEAEKAKRMKEWMKRIKTTASEDEQEEMANAIITILQSWIFS

SEQ ID NO: 23 - protein, 100 aa, Synthetic construct
  source 1..100: note = Neo_2/15_ N40S_D15S
PKKKIQLHAEHALYSALMILNIVKTNSPPAEEKLEDYAFSFELILEEIARLFESGDQKDEAEKAKRMKEWMKRIKTTASEDEQEEMANAIITILQSWIFS

SEQ ID NO: 24 - protein, 100 aa, Synthetic construct
  source 1..100: note = Neo_2/15_ H8R_D15S
PKKKIQLRAEHALYSALMILNIVKTNSPPAEEKLEDYAFNFELILEEIARLFESGDQKDEAEKAKRMKEWMKRIKTTASEDEQEEMANAIITILQSWIFS

SEQ ID NO: 25 - protein, 100 aa, Synthetic construct
  source 1..100: note = Neo_2/15_ H11F_D15S
PKKKIQLHAEFALYSALMILNIVKTNSPPAEEKLEDYAFNFELILEEIARLFESGDQKDEAEKAKRMKEWMKRIKTTASEDEQEEMANAIITILQSWIFS

SEQ ID NO: 26 - protein, 116 aa, Synthetic construct
  source 1..116: note = Daclizumab heavy chain variable region (VH)
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 27 - protein, 5 aa, Synthetic construct
  source 1..5: note = Daclizumab CDR-H1
SYRMH

SEQ ID NO: 28 - protein, 17 aa, Synthetic construct
  source 1..17: note = Daclizumab CDR-H2
YINPSTGYTEYNQKFKD

SEQ ID NO: 29 - protein, 7 aa, Synthetic construct
  source 1..7: note = Daclizumab CDR-H3
GGGVFDY

SEQ ID NO: 30 - protein, 10 aa, Synthetic construct
  source 1..10: note = Daclizumab CDR-L1
SASSSISYMH

SEQ ID NO: 31 - protein, 7 aa, Synthetic construct
  source 1..7: note = Daclizumab CDR-L2
TTSNLAS

SEQ ID NO: 32 - protein, 9 aa, Synthetic construct
  source 1..9: note = Daclizumab CDR-L3
HQRSTYPLT

SEQ ID NO: 33 - protein, 30 aa, Synthetic construct
  source 1..30: note = Daclizumab FR-H1
QVQLVQSGAEVKKPGSSVKVSCKASGYTFT

SEQ ID NO: 34 - protein, 14 aa, Synthetic construct
  source 1..14: note = Daclizumab FR-H2
WVRQAPGQGLEWIG

SEQ ID NO: 35 - protein, 17 aa, Synthetic construct
  source 1..17: note = Daclizumab FR-H3
YINPSTGYTEYNQKFKD

SEQ ID NO: 36 - protein, 11 aa, Synthetic construct
  source 1..11: note = Daclizumab FR-H4
WGQGTLVTVSS

SEQ ID NO: 37 - protein, 22 aa, Synthetic construct
  source 1..22: note = Daclizumab FR-L1
DIQMTQSPSTLSAVGDRVTITC

SEQ ID NO: 38 - protein, 15 aa, Synthetic construct
  source 1..15: note = Daclizumab FR-L2
WYQQKPGKAPKLLIY

SEQ ID NO: 39 - protein, 32 aa, Synthetic construct
  source 1..32: note = Daclizumab FR-L3
GVPARFSGSGSGTEFTLTISSLQPDDFATYYC

SEQ ID NO: 40 - protein, 10 aa, Synthetic construct
  source 1..10: note = Daclizumab FR-L4
FGQGTKVEVK

SEQ ID NO: 41 - protein, 106 aa, Synthetic construct
  source 1..106: note = Daclizumab light chain variable region (VL)
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 42 - protein, 5 aa, Synthetic construct
  source 1..5: note = Daclizumab CDR-H1 variant 1
GYRMH

SEQ ID NO: 43 - protein, 5 aa, Synthetic construct
  source 1..5: note = Daclizumab CDR-H1 variant 2
SYRMH

SEQ ID NO: 44 - protein, 17 aa, Synthetic construct
  source 1..17: note = Daclizumab CDR-H2 variant
WINPNSGGTNYAQKFQG

SEQ ID NO: 45 - protein, 10 aa, Synthetic construct
  source 1..10: note = Daclizumab CDR-L1 variant
RASQSVSYLH

SEQ ID NO: 46 - protein, 7 aa, Synthetic construct
  source 1..7: note = Daclizumab CDR-L2 variant
TTSNRAT

SEQ ID NO: 47 - protein, 127 aa, Synthetic construct
  source 1..127: note = HUMAX-TAC heavy chain variable region (VH)
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 48 - protein, 119 aa, Synthetic construct
  source 1..119: note = HUMAX-TAC light chain variable region (VL)
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 49 - protein, 20 aa, Synthetic construct
  source 1..20: note = GS linker
GGGGSGGGGSGGGGSGGGGS

SEQ ID NO: 50 - protein, 15 aa, Synthetic construct
  source 1..15: note = GS linker
GGGGSGGGGSGGGGS

SEQ ID NO: 51 - protein, 5 aa, Synthetic construct
  source 1..5: note = GS linker
GGGGS

SEQ ID NO: 52 - protein, 6 aa, Synthetic construct
  source 1..6: note = GS linker
GGGGGG

SEQ ID NO: 53 - protein, 8 aa, Synthetic construct
  source 1..8: note = GS linker
GGGGGGGG

SEQ ID NO: 54 - protein, 4 aa, Synthetic construct
  source 1..4: note = Rigid linker
EAAK

SEQ ID NO: 55 - protein, 8 aa, Synthetic construct
  source 1..8: note = Rigid linker
EAAKEAAK

SEQ ID NO: 56 - protein, 12 aa, Synthetic construct
  source 1..12: note = Rigid linker
EAAKEAAKEAAK

SEQ ID NO: 57 - protein, 15 aa, Synthetic construct
  source 1..15: note = Rigid linker
EAAAKEAAAKEAAAK

SEQ ID NO: 58 - protein, 46 aa, Synthetic construct
  source 1..46: note = Rigid linker
AEAAAKEAAAKEAAAKEAAAKALEAEAAAKEAAAKEAAAKEAAAKA

SEQ ID NO: 59 - protein, 12 aa, Synthetic construct
  source 1..12: note = Rigid linker
AEAAAKEAAAKA

SEQ ID NO: 60 - protein, 5 aa, Synthetic construct
  source 1..5: note = Rigid linker
PAPAP

SEQ ID NO: 61 - protein, 19 aa, Synthetic construct
  VARIANT 12: note = X is serine or alanine
  source 1..19: note = Neo-2/15 mutant D1
KIQLHAEHALYXALMILNI

SEQ ID NO: 62 - protein, 19 aa, Synthetic construct
  source 1..19: note = Neo-2/15 mutant D1
KIQLHAEHALYDALMILNI

SEQ ID NO: 63 - protein, 19 aa, Synthetic construct
  source 1..19: note = Neo-2/15 mutant D2
KDEAEKAKRMKEWMKRIKT

SEQ ID NO: 64 - protein, 22 aa, Synthetic construct
  source 1..22: note = Neo-2/15 mutant D3
LEDYAFNFELILEEIARLFESG

SEQ ID NO: 65 - protein, 22 aa, Synthetic construct
  VARIANT 7: note = X is serine or alanine
  VARIANT 11: note = X is serine or alanine
  source 1..22: note = Neo-2/15 mutant D3
LEDYAFXFELXLEEIARLFESG

SEQ ID NO: 66 - protein, 21 aa, Synthetic construct
  VARIANT 16: note = X is glutamic acid, alanine, or histidine
  source 1..21: note = Neo-2/15 mutant D4
EDEQEEMANAIITILXSWIFS

SEQ ID NO: 67 - protein, 10 aa, Synthetic construct
  source 1..10: note = Linker 1
VKTNSPPAEE

SEQ ID NO: 68 - skipped

SEQ ID NO: 69 - protein, 561 aa, Synthetic construct
  source 1..561: note = Daclizumab (anti-CD25) VH x huIgG1 (G1m17 LALA-GA) x (G4S)3 #1 x Neo215_D15S_Q95E
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPKKKIQLHAEHALYSALMILNIVKTNSPPAEEKLEDYAFNFELILEEIARLFESGDQKDEAEKAKRMKEWMKRIKTTASEDEQEEMANAIITILESWIFS

SEQ ID NO: 70 - protein, 213 aa, Synthetic construct
  source 1..213: note = Daclizumab (anti-CD25) VL x kappa (huIGKC)
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 330 aa, Synthetic construct
  source 1..330: note = Human IgG1 (LALAGA)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 107 aa, Synthetic construct [sequence omitted]

SEQ ID NO: 73 - protein, 446 aa, Synthetic construct
  source 1..446: note = Reduced/no effector function Daclizumab heavy chain (IgG1 LALAGA)
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 563 aa, Synthetic construct
  source 1..563: note = Neo215_N40S_I44S_Q95E x (G4S)3 #1 x reduced/no effector function daclizumab (anti-CD25) VH x huIgG1 (G1m17 LALA-GA)
GSPKKKIQLHAEHALYDALMILNIVKTNSPPAEEKLEDYAFSFELSLEEIARLFESGDQKDEAEKAKRMKEWMKRIKTTASEDEQEEMANAIITILESWIFSGGGGSGGGGSGGGGSQVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 100 aa, Synthetic construct
  source 1..100: note = Neo_2/15_D15S_Q95K
PKKKIQLHAEHALYSALMILNIVKTNSPPAEEKLEDYAFNFELILEEIARLFESGDQKDEAEKAKRMKEWMKRIKTTASEDEQEEMANAIITILKSWIFS

SEQ ID NO: 76 - protein, 100 aa, Synthetic construct
  source 1..100: note = Neo_2/15_D15S_Q95T
PKKKIQLHAEHALYSALMILNIVKTNSPPAEEKLEDYAFNFELILEEIARLFESGDQKDEAEKAKRMKEWMKRIKTTASEDEQEEMANAIITILTSWIFS

SEQ ID NO: 77 - protein, 100 aa, Synthetic construct
  source 1..100: note = Neo_2/15_D15S_Q95Y
PKKKIQLHAEHALYSALMILNIVKTNSPPAEEKLEDYAFNFELILEEIARLFESGDQKDEAEKAKRMKEWMKRIKTTASEDEQEEMANAIITILYSWIFS

SEQ ID NO: 78 - protein, 100 aa, Synthetic construct
  source 1..100: note = Neo_2/15_ N40G_I44S_Q95E
PKKKIQLHAEHALYDALMILNIVKTNSPPAEEKLEDYAFGFELSLEEIARLFESGDQKDEAEKAKRMKEWMKRIKTTASEDEQEEMANAIITILESWIFS

SEQ ID NO: 79 - protein, 100 aa, Synthetic construct
  source 1..100: note = Neo_2/15_ N40S_I44T_Q95E
PKKKIQLHAEHALYDALMILNIVKTNSPPAEEKLEDYAFSFELTLEEIARLFESGDQKDEAEKAKRMKEWMKRIKTTASEDEQEEMANAIITILESWIFS

SEQ ID NO: 80 - protein, 100 aa, Synthetic construct
  source 1..100: note = Neo_2/15_ N40S_I44Y_Q95E
PKKKIQLHAEHALYDALMILNIVKTNSPPAEEKLEDYAFSFELYLEEIARLFESGDQKDEAEKAKRMKEWMKRIKTTASEDEQEEMANAIITILESWIFS

SEQ ID NO: 81 - protein, 97 aa, Synthetic construct
  source 1..97: note = Neo_2/15_ N40S_I44Y_Q95E (-3AA)
KIQLHAEHALYDALMILNIVKTNSPPAEEKLEDYAFSFELYLEEIARLFESGDQKDEAEKAKRMKEWMKRIKTTASEDEQEEMANAIITILESWIFS

SEQ ID NO: 82 - protein, 100 aa, Synthetic construct
  source 1..100: note = Neo_2/15_ N40S_I44N_Q95E
PKKKIQLHAEHALYDALMILNIVKTNSPPAEEKLEDYAFSFELNLEEIARLFESGDQKDEAEKAKRMKEWMKRIKTTASEDEQEEMANAIITILESWIFS

SEQ ID NO: 83 - protein, 68 aa, Synthetic construct
  source 1..68: note = Rigid linker
  REGION 21..68: note = Amino acids may be present or absent in "AP" pairs
APAPAPAPAPAPAPAPAPAPAPAPAPAPAPAPAPAPAPAPAPAPAPAPAPAPAPAPAPAPAPAPAPAP

(19) United States
(12) Patent Application Publication
(10) Pub. No.: US 2025/0361308 A1
(43) Pub. Date: Nov. 27, 2025

(54) NEO-2/15 Variants and Uses Thereof for Preferentially Stimulating T-Regulatory Cells

(71) Applicant: Neurogene Inc., New York, NY (US)

(72) Inventors: Alex C. Chen, New York, NY (US); Alfredo Quijano Rubio, New York, NY (US); Ryan Swanson, New York, NY (US); Matthew James Walker, New York, NY (US)

(73) Assignee: Neurogene Inc., New York, NY (US)

(21) Appl. No.: 18/873,939

(22) PCT Filed: Jun. 30, 2023
(86) PCT No.: PCT/US2023/069457

§ 371 Date: Dec. 11, 2024

Related U.S. Application Data
(60) Provisional application No. 63/357,844, filed on Jul. 1, 2022.
(60) Provisional application No. 63/395,437, filed on Aug. 5, 2022.

(51) Int. Cl. C07K 16/28; A61K 38/00; A61P 37/06; C07K 14/55

(52) U.S. Cl. CPC C07K 16/2866; A61P 37/06; C07K 14/55; C07K 16/2896; A61K 38/00; C07K 2317/52; C07K 2317/565; C07K 2317/622; C07K 2317/92; C07K 2319/00

(57) ABSTRACT

Provided herein are polypeptides comprising a Neo-2/15 mutant, polypeptides comprising a Neo-2/15 mutant and an agent for targeting T-regulatory cells, and methods of their use for treating disease. Related nucleic acids, vectors, host cells, pharmaceutical compositions and kits are also providing. Methods of making the polypeptides, as well as methods of treating a subject in need thereof are also provided, as well as CD25 antigen binding proteins.

### CROSS-REFERENCE TO RELATED APPLICATIONS

This application claims the benefit of priority of U.S. Provisional Application Nos. 63/357,844, filed Jul. 1, 2022, and 63/395,437, filed Aug. 5, 2022, each of which is incorporated by reference herein in its entirety for any purpose.

### SEQUENCE LISTING

This application contains a Sequence Listing that has been submitted electronically in XML format and is hereby incorporated by reference in its entirety. Said XML copy, created on Jun. 21, 2023, is named “2023-06-21_01267-0005-00PCT_ST26” and is 95,337 bytes in size.

### BACKGROUND

Regulatory T cells (Tregs) are crucial for maintaining immune tolerance and their dysfunction contributes to inflammatory and autoimmune conditions. IL-2 is a pleiotropic cytokine that activates both immunosuppressive Tregs and inflammatory cells, including NK cells, cytotoxic T cells, and helper T cells. Although low dose IL-2 therapy effectively expands Tregs, this intervention is limited by coincident stimulation of conventional T cells and natural killer cells. New therapies for expanding Treg cells without concomitant stimulation of T cells and natural killer cells are needed.

### SUMMARY

Provided herein are polypeptides comprising a Neo-2/15 mutant, polypeptides comprising a Neo-2/15 mutant and an agent for targeting T-regulatory cells, and methods of their use for treating disease.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises a a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H), and at least one additional substitution selected from:
a) a serine or alanine in place of aspartic acid at position 15 (D15S or D15A);
b) an alanine or serine or glycine in place of asparagine at position 40 (N40A or N40S or N40G); and
c) an alanine or serine or asparagine or threonine or tyrosine in place of isoleucine at position 44 (I44A or I44S or I44N or I44T or I44Y);
   wherein the first amino acid of SEQ ID NO:2 is designated position 1, and the first amino acid of SEQ ID NO: 1 is designated position 4.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises a glutamic acid, an alanine, or a histidine in place of glutamine at position 95 (Q95E or Q95A or Q95H), and at least one additional substitution selected from:
a) a serine or alanine in place of aspartic acid at position 15 (D15S or D15A);
b) an alanine or serine in place of asparagine at position 40 (N40A or N40S); and
c) an alanine or serine in place of isoleucine at position 44 (I44A or I44S);
   wherein the first amino acid of SEQ ID NO:2 is designated position 1, and the first amino acid of SEQ ID NO: 1 is designated position 4.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises a serine or an alanine in place of aspartic acid at position 15 (D15S or D15A), and at least one additional substitution selected from:
a) a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H);
b) an arginine in place of histidine at position 8 (H8R);
c) a phenylalanine in place of histidine at position 11 (H11F);
d) a lysine in place of tyrosine at position 14 (Y14K);
e) an alanine or serine in place of asparagine at position 40 (N40A or N40S); and
f) an alanine or serine or tyrosine in place of isoleucine at position 44 (I44A or I44S or I44Y),
   wherein the first amino acid of SEQ ID NO: 1 is designated position 4 and the first amino acid of SEQ ID NO:2 is designated position 1.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises a serine or an alanine in place of aspartic acid at position 15 (D15S or D15A), and at least one additional substitution selected from:
a) a glutamic acid, alanine or histidine in place of glutamine at position 95 (Q95E, Q95A or Q95H);
b) an arginine in place of histidine at position 8 (H8R);
c) a phenylalanine in place of histidine at position 11 (H11F);
d) a lysine in place of tyrosine at position 14 (Y14K);
e) an alanine or serine in place of asparagine at position 40 (N40A or N40S); and
f) an alanine or serine in place of isoleucine at position 44 (I44A or I44S),
   wherein the first amino acid of SEQ ID NO: 1 is designated position 4 and the first amino acid of SEQ ID NO:2 is designated position 1.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises:
a) a phenylalanine in place of histidine at position 11 (H11F);
b) an alanine or serine in place of asparagine at position 40 (N40A or N40S); and
c) an alanine or serine or tyrosine in place of isoleucine at position 44 (I44A or I44S or I44Y),
   wherein the first amino acid of SEQ ID NO:1 is designated position 4 and the first amino acid of SEQ ID NO:2 is designated position 1.

In some embodiments, a polypeptide is provided comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises:
a) a phenylalanine in place of histidine at position 11 (H11F);
b) an alanine or serine in place of asparagine at position 40 (N40A or N40S); and
c) an alanine or serine in place of isoleucine at position 44 (I44A or I44S),
   wherein the first amino acid of SEQ ID NO: 1 is designated position 4 and the first amino acid of SEQ ID NO:2 is designated position 1.

In some embodiments, a polypeptide is provided comprising a Neo-2/15 mutant, wherein the Neo-2/15 mutant comprises domains D1, D2, D3, and D4; wherein:
(a) D1 comprises the amino acid sequence: KIQLHAEHALYX₁₅ALMILNI (SEQ ID NO: 61) and D3 comprises the amino acid sequence LEDYAFNFELILEEIARLFESG (SEQ ID NO:64); or
(b) D1 comprises the amino acid sequence: KIQLHAEHALYDALMILNI (SEQ ID NO: 62 and D3 comprises the amino acid sequence LEDYAFX₄₀FELX₄₄LEEIARLFESG (SEQ ID NO:65); and
D2 comprises an amino acid sequence at least 8 amino acids in length;
D4 comprises the amino acid sequence EDEQEEMANAIITILX₉₅SWIFS (SEQ ID NO: 66); wherein:
(i) D1, D2, D3 and D4 may be in any order in the Neo-2/15 mutant;
(ii) amino acid linkers may be present between any of the domains (“domain linkers”);
(iii) X₁₅ is serine or alanine; X₉₅ is glutamic acid, alanine, or histidine; X₄₀ is serine or alanine; and X₄₄ is serine or alanine or wherein X₁₅ is serine or alanine; X₉₅ is glutamic acid, lysine, arginine, threonine, serine, tyrosine, alanine, or histidine; X₄₀ is serine or alanine or glycine; and X₄₄ is serine or alanine or asparagine or threonine or tyrosine;
(iv) wherein the Neo-2/15 mutant contains a total of no more than ten, no more than nine, no more than eight, no more than seven, no more than six, no more than five, no more than four, no more than three, no more than two, no more than one, or zero substitutions at amino acid positions not designated as X.

### BRIEF DESCRIPTION OF THE FIGURES

FIGS. 1A-B provides a graph of the attenuation of STAT5 signaling for the Neo-2/15 mutants, Neo-2/15_D15S_Q95E and Neo-2/15_D15A_Q95A. The percentage of CD8+ cells (triangles), CD4+ cells (squares) and Treg cells (circles) that demonstrate STAT5 phosphorylation after stimulation with Neo-2/15_D15S_Q95E (solid line, filled shapes) and Neo-2/15 (dotted line, open shapes) is presented in FIG. 1A and the percentage of CD8+ (triangles), CD4+ (squares) and Treg cells (circles) that demonstrate STAT5 phosphorylation after stimulation with titrations of Neo-2/15_D15A_Q95A (solid line, filled shapes) and Neo-2/15 (dotted line, open shapes) is presented in FIG. 1B.

FIGS. 2A-C provide a representation of fusion protein formats. In FIG. 2A, a Neo-2/15 mutant and scFv are both placed on opposite termini of the Fc. In FIG. 2B, a Neo-2/15 mutant is linked to the N terminus of a heavy chain of an antibody. In FIG. 2C, a Neo-2/15 mutant is linked to the C-terminus of the heavy chain of an antibody.

FIGS. 3A-F provide a graph of STAT5 signaling for Neo-2/15 and Neo-2/15 mutants fused to an anti-CD25 scFv. The percentage of all T cells (squares), CD8+ cells (triangles), CD4+ cells (squares) and Treg cells (circles) that demonstrate STAT5 phosphorylation after stimulation with titrations of scFv fusion proteins (solid line, filled shapes) and human IL-2 (dotted line, open shapes) is provided. FIG. 3A provides Neo-2/15 fused to anti-CD25 ScFV; FIG. 3B provides Neo-2/15_D15S_Q95E fused to anti-CD25 ScFV; FIG. 3C provides Neo-2/15_D15A_Q95A fused to anti-CD25 ScFV; FIG. 3D provides Neo-2/15_D15A_Q95H fused to anti-CD25 ScFV; FIG. 3E provides Neo-2/15_L13R_D15A_L17E_Q95H fused to anti-CD25 ScFV; and FIG. 3F provides Neo-2/15_L13R_L17E_N40A_Q95H fused to anti-CD25 ScFV.

FIGS. 4A-B provide a graph of STAT5 signaling for Neo-2/15 fused to anti-CD25 ScFV and Neo-2/15_D15S_Q95E fused to anti-CD25 ScFV in a second patient sample. The percentage of all T cells (circles), CD8+ cells (upright triangles), CD4+ cells (delta triangle) and Treg cells (squares) that demonstrate STAT5 phosphorylation after stimulation with titrations of the scFv fusion proteins (solid line, filled shapes) and human IL-2 control at 1 nM (dotted line) is provided. FIG. 4A provides Neo-2/15 fused to anti-CD25 ScFV and FIG. 4B provides Neo-2/15_D15S_Q95E fused to anti-CD25 ScFv.

FIGS. 5A-B provide a graph of STAT5 signaling for the Neo-2/15 mutant D15S_Q95E unfused (5A) and fused to an anti-CD25 ScFv (5B). The percentage of CD8+ cells (triangles), CD4+ cells (squares) and Treg cells (circles) that demonstrate STAT5 phosphorylation after stimulation with titrations of the test articles is provided.

FIGS. 6A-C provide a graph of STAT5 signaling for the Neo-2/15 mutant D15S_Q95E fused to an anti-CD25 ScFv (6A); fused to a full length antibody at the C terminus (6B); and fused to a full length antibody at the N terminus (6C). The percentage of all T cells (diamonds), CD8+ cells (triangles), CD4+ cells (squares) and Treg cells (circles) that demonstrate STAT5 phosphorylation after stimulation with titrations of the test articles is provided.

FIGS. 7A-D provide a graph of STAT5 signaling for Neo-2/15 mutants fused to the C-terminus of the heavy chain of an anti-CD25 antibody (as shown in FIG. 2C). The percentage of CD8+ cells (triangles), CD4+ cells (squares) and Treg cells (circles) that demonstrate STAT5 phosphorylation after stimulation with titrations of scFv fusion proteins (solid line, filled shapes) and human IL-2 (dotted line, open shapes) is provided. FIG. 7A provides a fusion protein with Neo-2/15_H11F_N40S_I44S; 7B provides a fusion protein with Neo-2/15_Y14K_D15S; 7C provides a fusion protein with Neo-2/15_N40S_Q95E; and 7D provides a fusion protein with Neo-2/15_D15S_Q95E.

FIGS. 8A-D provide a graph of STAT5 signaling for Neo-2/15 mutants fused to the C-terminus of the heavy chain of an anti-CD25 antibody (as shown in FIG. 2C). The percentage of CD8+ cells (triangles), CD4+ cells (squares) and Treg cells (circles) that demonstrate STAT5 phosphorylation after stimulation with titrations of scFv fusion proteins (solid line, filled shapes) and human IL-2 (dotted line, open shapes) is provided. This experiment repeats the experiment of FIG. 7 with a different patient sample. FIG. 7A provides a fusion protein with Neo-2/15_H11F_N40S_I44S; 7B provides a fusion protein with Neo-2/15_Y14K_D15S; 7C provides a fusion protein with Neo-2/15_N40S_Q95E; and 7D provides a fusion protein with Neo-2/15_D15S_Q95E.

FIGS. 9A-B provide a graph of STAT5 signaling for Neo-2/15_N40S_I44S_Q95E fused to the N-terminus of the heavy chain of an anti-CD25 antibody (as shown in FIG. 2C) for two different patient samples (9A, 9B). The percentage of CD8+ (triangles), CD4+ (squares) and Treg cells (circles) that demonstrate STAT5 phosphorylation after stimulation with titrations of scFv fusion proteins (solid line, filled shapes) and human IL-2 (dotted line, open shapes) is provided.

FIGS. 10A-E provide graph showing Treg expansion in an in vivo mice study. The mice were engrafted with human CD34+ hematopoietic stem cells and have >25% human CD45+ cells in peripheral blood. At Days −1 and 13, mice were injected intraperitoneally with 5 mg of human IgG. On Days 0, 7, 14, and 21, mice were injected intraperitoneally with 15-45 ug of test articles. On Days −1, 6, 13, and 20, 100 μL of whole blood was collected by retro-orbital bleed in K2EDTA. FIGS. 10A and 10B demonstrate the expansion of Tregs over time measured as a ratio of CD4+ cells with stimulation by test articles at the indicated dose level. FIG. 10C demonstrates the expansion of NK cells over time with stimulation by test articles at the indicated dose level. FIGS. 10D and 10E demonstrate the Treg Foxp3 MFI over time with stimulation by test articles at the indicated dose level. The test articles are vehicle control (open circle), distal fused Treg agonist (TRA; open squares), and proximal fused TRA. The distal fused TRA is Neo-2/15_D15S_Q95E fused to the C-terminus of the heavy chain of anti-CD25 antibody. The proximal fused TRA is Neo-2/15_N40S_I44S_Q95E fused to the N-terminus of the heavy chain of an anti-CD25 antibody.

FIGS. 11A-G provide a graph of STAT5 signaling for the C-terminally fused Neo-2/15 fusion proteins D15S_Q95K (11A); D15S_Q95T (11B); D15S_Q95Y (11C); N40G_I44S_Q95E (11D); N40S_I44T_Q95E (11E); N40S_I44Y_Q95E (11F); and N40S_I44N_Q95E (11G). The pSTAT5 mean fluorescence index (MFI) of CD8+ cells (circles), CD4+ cells (squares) and Treg cells (triangles) is provided.

### DETAILED DESCRIPTION

Neo-2/15 is a computationally designed, hyperstable IL-2Rα-independent agonist of the IL-2 and IL-15 receptors, which share IL-2R-beta (IL-2Rβ or CD122) and IL-2R-gamma (IL-2Rγ or CD132) signaling subunits. Neo-2/15 has been assigned CAS registry number 2407798-79-0. NL-201 was developed from Neo-2/15 by introducing a cysteine residue at position 62 for site-specific conjugation of an unbranched 40 kDa polyethylene glycol (PEG) molecule and is being developed as a potent activator of CD8+ T cells, CD4+ T cells, and natural killer (NK) cells for cancer immunotherapy.

The present inventors have surprisingly discovered that certain Neo-2/15 mutants, when targeted to T-regulatory cells (Tregs), are able to preferentially stimulate and expand Tregs as compared to non Tregs. In particular, by mutating select amino acids of Neo-2/15 to attenuate its binding to the medium affinity IL-2 receptor, IL-2Rβγ_{c}, and attaching the resultant Neo-2/15 mutant to an agent that targets Tregs, the present inventors have created potent selective T-regulatory cell agonists, also referred to herein as TRAs. Unlike other Treg agonists based on IL-2, these TRAs are comprised of at least two distinct components, the Treg targeting domain and the attenuated Neo-2/15 mutant. By decoupling the Treg binding site from the IL-2Rβ and IL-2γ_{c} binding sites, orientation and proximity to the binding site can be manipulated and optimized.

### Regulatory T Cells (Tregs)

Regulatory T-cells (Tregs) are naturally occurring CD4+CD25+FOXP3+ T lymphocytes that comprise about 5-10% of the circulating CD4+ T cell population, act to dominantly suppress autoreactive lymphocytes, and control innate and adaptive immune responses. Tregs achieve this suppression, at least in part, by inhibiting the proliferation, expansion, and effector activity of T effector cells (Teffs). Whereas Foxp3 is the accepted marker of Treg cells, it is difficult to use the Foxp3 marker to isolate cells for functional studies. Cells that are CD4(+)CD25(+)CD127(low/−) have been shown to express the highest level of Foxp3 and have the strongest correlation with CD4(+)CD25(+)Foxp3(+) T cells (See Yu et al., Inflammation, 2021 December; 35(6):1773-80). The present inventors use CD4(+)CD25(+)CD127(low/−) as identifying characteristics for Tregs. Teffs are conventional T cells that have effector functions (e.g., cytokine secretion, cytotoxic activity, and the like) to increase immune responses by virtue of their expression of one or more T cell receptors. Teffs for the purposes of the present invention are defined as CD4+ and CD8+ T cells that are not Tregs.

Increasing the number of Tregs, increasing Treg activity, and/or decreasing Treg cell death (e.g., apoptosis) is known to be useful for suppressing unwanted immune reactions associated with a range of immune disorders and inflammation. Treatments with a Treg agonist will ideally preferentially enhance Tregs with minimal or no activation of Teffs or other cells that may worsen inflammation. The examples and teachings provided herein demonstrate the surprising and unexpected result that polypeptides comprising targeted Neo-2/15 mutants can be used to selectively and potently activate Tregs over Teffs, which demonstrates that the polypeptides can be used to treat or ameliorate diseases and conditions that would benefit from the suppression of immune response, such as autoimmune disease and disease and conditions associated with inflammation.

Treg agonists (TRAs)

Provided herein, inter alia, are TRAs that preferentially stimulate Treg cells. As used herein “preferentially stimulates T regulatory cells” means that the TRAs promote the proliferation, survival, activation and/or function of Tregs over non Tregs. In some aspects, the TRAs preferentially stimulate Tregs relative to Teffs or NK cells. In some embodiments, a TRA comprises at least one Neo-2/15 mutant provided herein attached to a targeting agent that binds an antigen on the surface of Treg cells.

Methods of measuring the ability to preferentially stimulate Tregs can be measured by flow cytometry of peripheral blood leukocytes, in which there is an observed increase in the percentage of Tregs among total CD4+ T cells, an increase in percentage of Tregs among total CD8+ T cells, an increase in percentage of Tregs relative to NK cells, and/or a greater increase in the expression level of CD25 on the surface of Tregs relative to the increase of CD25 expression on other T cells.

In some embodiments, TRAs that preferentially stimulate Treg cells increase the percentage of Tregs among total CD4+ T cells in a subject or a peripheral blood sample by at least 10%, at least 20%, at least 30%, at least 40%, at least 50%, at least 60%, at least 70%, at least 80%, at least 90%, at least 100%, at least 150%, at least 200%, or at least 300%. In some aspects, potent Treg expansion is measured by at least 2 times higher percentage of Tregs in the total population of CD4+ cells when treated with a TRA as compared to a vehicle control.

In some embodiments, methods of identifying TRAs of the present invention include measuring the ability of the TRA to promote or stimulate STAT5 phosphorylation in Tregs as compared to Teffs. Exemplary TRAs will have a significantly reduced ability to promote or stimulate STAT5 phosphorylation in Teffs as compared to Neo-2/15 and/or IL-2 while maintaining the ability to promote or stimulate STAT5 phosphorylation in Tregs.

In some embodiments, exemplary TRAs of the present invention do not substantially activate or induce proliferation of Teffs. In some aspects, activation or proliferation of Teffs is measured using a STAT5 assay as described herein. In some embodiments, TRAs preferably have a maximal signaling in Teffs cells that is less than 50%, less than 40%, less than 30%, or less than 20% of the maximal signaling resulting from IL-2 stimulation (e.g., at concentrations of up to 10 nM). Maximal signaling in Tregs, however, should preferably be at least 40%, at least 50%, at least 60%, or at least 70% of the maximal signaling resulting from IL-2 stimulations, with higher maximal Treg signaling indicating a more potent TRA. In some preferred embodiments, the EC50 of the Treg % pSTAT5+ curve should be below 10 nM, below 1 nM, below 500 pM, below 250 pM, or even below 100 pM, with a lower Treg EC50 also indicating a more potent TRA. In some embodiments, the EC50 of the Treg % pSTAT5+ curve for a TRA provided herein is below 1 nM, with a maximal signaling in Tregs of greater than 50% of the maximal signaling resulting from IL-2 stimulation.

### Polypeptides

The present invention provides, inter alia, Neo-2/15 mutants. Exemplary Neo-2/15 mutants have attenuated binding to IL-2Rβγ_{c} as compared to Neo-2/15. Such Neo-2/15 mutants are optionally linked (e.g., by fusion or attached by chemical/enzymatic conjugation) to a targeting agent that binds an antigen on the surface of Treg cells. An exemplary antigen for use in the present invention is CD25 or CD39. In exemplary embodiments of the present invention, polypeptides comprising Neo-2/15 mutants that are linked to a targeting agent that binds an antigen on the surface of Treg cells are Treg agonists (TRAs). As used herein, when two polypeptides are “fused” it is meant that they are produced (e.g., translated) as a single contiguous polypeptide.

In some embodiments, Neo-2/15 mutants comprise an amino acid sequence having at least 80%, at least 85%, at least 90%, at least 95%, identity to Neo-2/15 and have one or more amino acid substitutions that reduce binding to IL-2Rβ and/or one or more amino acid substitutions that reduces binding to IL-2Rγ_{c}. In some embodiments, Neo-2/15 mutants comprise an amino acid sequence having at least 80%, at least 85%, at least 90%, at least 95%, identity to Neo-2/15 and one to five amino acid substitutions that reduce binding to IL-2Rβγ_{c} as compared to Neo-2/15.

In some embodiments, polypeptides of the present invention comprise a Neo-2/15 mutant, wherein the Neo-2/15 mutant comprises an amino acid sequence at least 79%, at least 80%, at least 85%, at least 90%, at least 95%, at least 96%, at least 97%, or at least 98% identical to SEQ ID NO: 1 (truncated Neo-2/15 without its 3 N-terminal amino acids) or SEQ ID NO:2 (Neo-2/15). Such Neo-2/15 mutants comprise 2-20, 2-15, 2-10, 2-5, 2-4 or 2-3 amino acid substitutions as compared to Neo-2/15. The amino acid sequences of exemplary Neo-2/15 mutants are set forth in SEQ ID NO:3-25 and 75-82. Particularly preferred is the amino acid sequence set forth in SEQ ID NO:3 or 4 comprising a Q95E, D15S substitution or SEQ ID NO:6 or 7 comprising a Q95E, N40S, and I44s substitution or SEQ ID NO:80 or 81 comprising a N40S, I44Y, and Q95E substitution. Although it may be advantageous to reduce the number of further mutations, the invention includes Neo-2/15 mutants having truncations or additional insertions, deletions, or substitutions in addition to those described herein, provided that said Neo-2/15 mutants, when targeted to Treg cells (e.g., by fusion to an anti-CD25 antibody such as in the manner described herein) maintain the activity of preferentially stimulating Tregs. To the extent that there are additions or deletions in the Neo-2/15 sequence, such additions or deletions are preferably not within the D1, D3, or D4 domains of the Neo-2/15 mutants (i.e., not within amino acids 4-22, 34-55, and 80-100, numbered according to SEQ ID NO:2). In some embodiments, to the extent that there are additional substitutions in Neo-2/15 (substitutions other than those at positions 8, 11, 14, 15, 40, 44, or 95, numbered according to SEQ ID NO:2), there are not more than 1-10, not more than 1-5, or not more than 1-3 such mutations within the amino acid sequences of the D1, D3 or D4 domains.

Included in the present invention are polypeptides comprising a Neo-2/15 mutant, wherein the Neo-2/15 mutant comprises an amino acid sequence at least 80%, at least 85%, or at least 90%, at least 91%, at least 92%, at least 93%, at least 94%, at least 95%, at least 96%, at least 97% or at least 98% identical to SEQ ID NO: 1 or SEQ ID NO:2 wherein the Neo-2/15 mutant comprises a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H), and at least one additional substitution selected from:
a) a serine or alanine in place of aspartic acid at position 15 (D15S or D15A);
b) an alanine or serine or glycine in place of asparagine at position 40 (N40A or N40S or N40G); and
c) an alanine or serine or asparagine or threonine or tyrosine in place of isoleucine at position 44 (I44A or I44S or I44N or I44T or I44Y);
   wherein the first amino acid of SEQ ID NO: 1 is designated position 4 and the first amino acid of SEQ ID NO:2 is designated position 1.

Included in the present invention are polypeptides comprising a Neo-2/15 mutant, wherein the Neo-2/15 mutant comprises an amino acid sequence at least 80%, at least 85%, or at least 90%, at least 91%, at least 92%, at least 93%, at least 94%, at least 95%, at least 96%, at least 97% or at least 98% identical to SEQ ID NO: 1 or SEQ ID NO:2 wherein the Neo-2/15 mutant comprises a glutamic acid, an alanine, or a histidine in place of glutamine at position 95 (Q95E or Q95A or Q95H), and at least one additional substitution selected from:
a) a serine or alanine in place of aspartic acid at position 15 (D15S or D15A);
b) an alanine or serine in place of asparagine at position 40 (N40A or N40S); and
c) an alanine or serine in place of isoleucine at position 44 (I44A or I44S)
   wherein the first amino acid of SEQ ID NO: 1 is designated position 4 and the first amino acid of SEQ ID NO:2 is designated position 1.

In some embodiments, the Neo2/15 mutant comprises a set of substitutions selected from Q95E and D15S; Q95E and D15A; Q95A and D15S; Q95A and D15A; Q95H and D15S; Q95H and D15A; Q95E, N40S AND I44S; Q95E, N40A AND I44A; Q95E, N40S AND I44A; Q95E, N40A AND I44S; Q95A, N40S AND I44S; Q95A, N40A AND I44A; Q95A, N40S AND I44A; Q95A, N40A and I44S; Q95H, N40S AND I44S; Q95H, N40A AND I44A; Q95H, N40S AND I44A; Q95H, N40A AND I44S; D15S and Q95K; D15S and Q95T; D15S and Q95Y; N40G, I44S, and Q95E; N40S, I44T, and Q95E; N40S, I44Y, and Q95E; and N40S, I44N, and Q95E.

In some such aspects, an additional 1-18, 1-10, 1-9, 1-8, 1-7, 1-6, 1-5, 1-4, 1-3, or 1-2 substitutions are made in addition to those noted above. In some aspects, no other substitutions are made. Exemplary additional substituents include, for example, a phenylalanine in place of histidine at position 11 (H11F), a lysine in place of tyrosine at position 14 (Y14K), an arginine in place of histidine at position 8 (H8R). In some embodiments, position 8, 11, and 14 are not substituted. In some aspects, the Neo-2/15 mutant comprises an alanine in place of aspartic acid at position 15. In some such aspects, the Neo-2/15 mutant comprises an arginine in place of leucine at position 13, and/or a glutamic acid in place of leucine at position 17. In some embodiments, if the Neo-2/15 mutant comprises a serine or alanine in place of aspartic acid at position 15, it does not comprise (i) a serine or alanine substitution in place of asparagine at position 40 or it does not comprise (ii) a serine or alanine substitution in place of isoleucine at position 44.

In some embodiments, if the Neo-2/15 mutant comprises a serine or alanine in place of aspartic acid at position 15, it does not comprise (i) a serine or alanine or glycine substitution in place of asparagine at position 40 and/or it does not comprise (ii) a serine or alanine or asparagine or threonine or tyrosine substitution in place of isoleucine at position 44. In some embodiments, if the Neo-2/15 mutant comprises a serine or alanine in place of aspartic acid at position 15, it does not comprise (i) a substitution at position 40 or (ii) a substitution at position 44. In some embodiments, if the Neo-2/15 mutant comprises a serine or alanine in place of aspartic acid at position 15, it does not comprise (i) a serine or alanine substitution in place of asparagine at position 40 and it does not comprise (ii) a serine or alanine substitution in place of isoleucine at position 44. In some embodiments, if the Neo-2/15 mutant comprises a serine or alanine in place of aspartic acid at position 15, it does not comprise (i) a substitution at position 40 and (ii) a substitution at position 44. In some embodiments, wherein the Neo-2/15 mutant comprises an alanine or serine in place of asparagine at position 40 and/or an alanine or serine in place of isoleucine at position 44, it comprises an acidic amino acid at position 15.

In some embodiments, the Neo-2/15 mutant comprises an alanine or serine or asparagine or threonine or tyrosine in place of isoleucine at position 44 (I44A or I44S or I44N or I44T or I44Y). In some embodiments, the Neo-2/15 mutant comprises an alanine or serine or glycine in place of asparagine at position 40 (N40A or N40S or N40G). In some embodiments, the Neo-2/15 mutant comprises a set of substitutions selected from Q95E and D15S; Q95E, D15S, and H11F; Q95E, N40S, and I44S; Q95E, N40S, I44S, and H11F; Q95E, N40S, I44S, Q95H, D15A, L17E, and L13R; and Y14K; Q95E, D15S, Y14K; Q95H and D15A; and Q95A and D15A. In some aspects, the Neo-2/15 mutant comprises additional substituents to the ones noted herein at positions 8, 11, 14, 15, 40, 44 and 95. In some aspects, the Neo-2/15 mutant does not comprise additional substituents to the ones noted herein at positions 8, 11, 14, 15, 40, 44 and 95.

Included in the present invention are polypeptides comprising a Neo-2/15 mutant wherein the polypeptide comprises an amino acid sequence at least 80%, at least 85%, or at least 90%, at least 91%, at least 92%, at least 93%, at least 94%, at least 95%, at least 96%, at least 97% or at least 98% identical to SEQ ID NO: 1 or SEQ ID NO:2, wherein the Neo-2/15 mutant comprises a serine or an alanine in place of aspartic acid at position 15 (D15S or D15A), and at least one additional substitution selected from:
a) a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H);
b) an arginine in place of histidine at position 8 (H8R);
c) a phenylalanine in place of histidine at position 11 (H11F);
d) a lysine in place of tyrosine at position 14 (Y14K);
e) an alanine or serine in place of asparagine at position 40 (N40A or N40S); and
f) an alanine or serine or tyrosine in place of isoleucine at position 44 (I44A or I44S or I44Y),
   wherein the first amino acid of SEQ ID NO: 1 is designated position 4 and the first amino acid of SEQ ID NO:2 is designated position 1. In some such aspects, an additional 1-18, 1-10, 1-9, 1-8, 1-7, 1-6, 1-5, 1-4, 1-3, or 1-2 substitutions are made in addition to those noted above. In some aspects, no other substitutions are made. In some such exemplary embodiments, if the Neo-2/15 mutant comprises a substitution at position 95, it does not comprise a substitution at position 40 and/or 44. In some such exemplary embodiments, if the Neo-2/15 comprises a substitution at position 40 or 44, it does not comprise a substitution at position 95, 11, or 8. In some such exemplary embodiments, if the Neo-2/15 mutant comprises a serine or alanine in place of aspartic acid at position 15, it does not comprise a substitution at position 14. In some exemplary embodiments, if the polypeptide comprises a serine or alanine in place of aspartic acid at position 15, it does not comprise a lysine substitution in place of tyrosine at position 14. In some such exemplary embodiments, if the polypeptide comprises a substitution at position 95, it does not comprise a substitution at position 8. In some such exemplary embodiments, if the polypeptide comprises a glutamic acid in place of glutamine at position 15, it does not comprise an arginine substitution in place of histidine at position 8.

Included in the present invention are polypeptides comprising a Neo-2/15 mutant wherein the polypeptide comprises an amino acid sequence at least 80%, at least 85%, or at least 90%, at least 91%, at least 92%, at least 93%, at least 94%, at least 95%, at least 96%, at least 97% or at least 98% identical to SEQ ID NO: 1 or SEQ ID NO:2, wherein the Neo-2/15 mutant comprises a serine or an alanine in place of aspartic acid at position 15 (D15S or D15A), and at least one additional substitution selected from:
g) a glutamic acid, alanine or histidine in place of glutamine at position 95 (Q95E, Q95A or Q95H);
h) an arginine in place of histidine at position 8 (H8R);
i) a phenylalanine in place of histidine at position 11 (H11F);
j) a lysine in place of tyrosine at position 14 (Y14K);
k) an alanine or serine in place of asparagine at position 40 (N40A or N40S); and 1) an alanine or serine in place of isoleucine at position 44 (I44A or I44S),
   wherein the first amino acid of SEQ ID NO: 1 is designated position 4 and the first amino acid of SEQ ID NO:2 is designated position 1. The polypeptide can comprise a set of substitutions selected from, for example, D15S and Y14K; N40S, I44S, and D15S; N40S, I44S, Y14K, and D15S; N40S and D15S; H8R and D15S; and H11F and D15S. In some such aspects, an additional 1-18, 1-10, 1-9, 1-8, 1-7, 1-6, 1-5, 1-4, 1-3, or 1-2 substitutions are made in addition to those noted above. In some aspects, no other substitutions are made. In some such exemplary embodiments, if the Neo-2/15 mutant comprises a substitution at position 95, it does not comprise a substitution at position 40 and/or 44. In some such exemplary embodiments, if the Neo-2/15 comprises a substitution at position 40 or 44, it does not comprise a substitution at position 95, 11, or 8. In some such exemplary embodiments, if the Neo-2/15 mutant comprises a serine or alanine in place of aspartic acid at position 15, it does not comprise a substitution at position 14. In some exemplary embodiments, if the polypeptide comprises a serine or alanine in place of aspartic acid at position 15, it does not comprise a lysine substitution in place of tyrosine at position 14. In some such exemplary embodiments, if the polypeptide comprises a substitution at position 95, it does not comprise a substitution at position 8. In some such exemplary embodiments, if the polypeptide comprises a glutamic acid in place of glutamine at position 15, it does not comprise an arginine substitution in place of histidine at position 8.

Included in the present invention are polypeptides comprising a Neo-2/15 mutant wherein the polypeptide comprises an amino acid sequence at least 80%, at least 85%, or at least 90%, at least 91%, at least 92%, at least 93%, at least 94%, at least 95%, at least 96%, at least 97% or at least 98% identical to SEQ ID NO: 1 or SEQ ID NO:2, wherein the polypeptide comprises:
a) a phenylalanine in place of histidine at position 11 (H11F);
b) an alanine or serine in place of asparagine at position 40 (N40A or N40S); and
c) an alanine or serine or tyrosine in place of isoleucine at position 44 (I44A or I44S or I44Y),
   wherein the first amino acid of SEQ ID NO: 1 is designated position 4 and the first amino acid of SEQ ID NO:2 is designated position 1. The Neo-2/15 mutant can comprise a set of substitutions selected from N40S, I44S, and H11F. In some aspects, the Neo-2/15 mutant comprises additional substituents to the ones noted herein at positions 11, 40 and 44. In some such embodiments, the Neo-2/15 mutant does not comprise a substitution at 1, 2, 3 or all 4 of positions 8, 14, 15, and 95, and any combinations thereof. In some aspects the polypeptide comprises a phenylalanine in place of histidine at position 11 (H11F); an alanine or serine in place of asparagine at position 40 (N40A or N40S); and an alanine or serine in place of isoleucine at position 44 (I44A or I44S).

In some exemplary embodiments, substitutions don't occur at 1, 2, 3, 4, 5, 6, 7, 8, 9, 10, 11, 12, 13, 14, 15, 16, 17, 18, 19, 20, 21, 22 or all 23 of positions 6, 7, 8, 10, 11, 13, 14, 17, 18, 33, 36, 37, 39, 43, 47, 84, 85, 88, 91, 92, 96, 98, or 99; and any combinations thereof. In some exemplary embodiments, substitutions don't occur at 1, 2, 3, 4, 5, 6, 7, 8, 9, 10, 11, 12, 13, 14, 15, 16, 17, 18, 19, or all 20 of positions 6, 7, 10, 13, 17, 18, 33, 36, 37, 39, 43, 47, 84, 85, 88, 91, 92, 96, 98, or 99. In some exemplary embodiments, substitutions don't occur at 1, 2, 3, 4, or all 4 of positions 91, 92, 96, and 99. In some exemplary embodiments, substitutions don't occur at 1, 2, 3, 4, or all 5 of positions 17, 91, 92, 96, and 99. In some exemplary embodiments, to the extent that a Neo-2/15 mutant has a substitution at a position other than positions 8, 11, 14, 15, 40, 44, or 95, it is a substitution that does not substantially interfere with binding of the Neo-2/15 mutant to IL-2Rγ_{c}.

In some exemplary embodiments, the Neo-2/15 mutant comprises three amino acids N-terminal and attached to the amino acid at position 4, wherein the amino acids are proline-lysine-lysine.

Neo-2/15 comprises 4 helical domains in the order D1-D3-D2-D4. D1 is from about amino acids 1-22 of Neo-2/15; D3 is from about amino acid 33-55 of Neo-2/15; D2 is from about amino acid 58-76 of Neo-2/15; and D4 is from about amino acid 80-100 of Neo-2/15. Although the Neo-2/15 mutants of the examples have the same domain order as Neo-2/15, the present invention also includes Neo-2/15 mutants that have undergone a re-ordering of the helical domains. The skilled artisan will understand that the domains can be re-ordered and still result in a similarly attenuated polypeptide that can be linked to a Treg targeting domain. In some embodiments, the domains are reordered by circular permutation, which creates a new N- and C-terminus. Re-ordering domains by circular permutation results in an order of domains including D4-D1-D2-D3, D3-D4-D1-D2, and D2-D3-D4-D1. The linkers between the domains may be altered to accommodate the re-ordering. Exemplary Neo-2/15 mutants comprise amino acid linkers between one or more of the domains. Such linkers are generally not involved in binding to IL-2Rβγ_{c} and function to connect the four domains. There is great variability permitted in the length of the linker and the identity of the linker amino acids. In various embodiments, the linkers can be of any length. In some aspects, the linkers are from 1 to 100 amino acids in length, such as 1-100, 1-90, 1-80, 1-70, 1-60, 1-50, 1-40, 1-30, 1-20, 1-10, 2-10 or 1-5 amino acids in length. The skilled practitioner can use the teachings in the art (see, for example, Silva et al., Nature, 2019 January; 565(7738):186-191) in combination with the teachings of the present specification to shift the domains and/or to construct linkers for connecting the domains while maintaining the desirable properties of the polypeptides. Included are embodiments wherein the order of the domains is D1-D3-D2-D4, wherein there is a first linker between domains D1 and D3, a second linker between domains D3 and D2, and a third linker between D2 and D4. In some aspects, the first linker is 10 amino acids in length, the second linker is 2 amino acids in length, and the third linker is 3 amino acids in length. An exemplary sequence for the first linker is VKTNSPPAEE (SEQ ID NO:67). An exemplary sequence for the second linker is DQ and an exemplary sequence for the third linker is TAS (SEQ ID NO: 68).

In some embodiments, a polypeptide of the present invention comprises a Neo-2/15 mutant, wherein the Neo-2/15 mutant comprises domains D1, D2, D3, and D4; wherein:
(a) D1 comprises the amino acid sequence: KIQLHAEHALYX₁₅ALMILNI (SEQ ID NO: 61) and D3 comprises the amino acid sequence LEDYAFNFELILEEIARLFESG (SEQ ID NO:64); or
(b) D1 comprises the amino acid sequence: KIQLHAEHALYDALMILNI (SEQ ID NO: 62) and D3 comprises the amino acid sequence LEDYAFX₄₀FELX₄₄LEEIARLFESG (SEQ ID NO:65); and
D2 comprises an amino acid sequence at least 8 amino acids in length;
D4 comprises the amino acid sequence EDEQEEMANAIITILX₉₅SWIFS (SEQ ID NO: 66); wherein:
(i) D1, D2, D3 and D4 may be in any order in the polypeptide;
(ii) amino acid linkers may be present between any of the domains (“domain linkers”);
(iii) X₁₅ is serine or alanine; X₉₅ is glutamic acid, alanine, or histidine; X₄₀ is serine or alanine; and X₄₄ is serine or alanine or X₁₅ is serine or alanine; X₉₅ is glutamic acid, lysine, arginine, threonine, serine, tyrosine, alanine, or histidine; X₄₀ is serine or alanine or glycine; and X₄₄ is serine or alanine or asparagine or threonine or tyrosine; wherein the polypeptide contains a total of no more than ten, no more than nine, no more than eight, no more than seven, no more than six, no more than five, no more than four, no more than three, no more than two, no more than one, or zero substitutions at amino acid positions not designated as X. In some embodiments, X₄₀ is serine or alanine; and X₄₄ is serine or alanine or tyrosine. In some embodiments, X₉₅ is glutamic acid, alanine, or histidine; X₄₀ is serine or alanine; and X₄₄ is serine or alanine.

In some embodiments D1 comprises the amino acid sequence: KIQLHAEHALYX₁₅ALMILNI (SEQ ID NO: 61) and D3 comprises the amino acid sequence LEDYAFNFELILEEIARLFESG (SEQ ID NO:64) and the histidine at position 8 of SEQ ID NO:61 is substituted. In some such embodiments, the substituent is phenylalanine.

In some embodiments, D1 comprises the amino acid sequence: KIQLHAEHALYX₁₅ALMILNI (SEQ ID NO: 61) and D3 comprises the amino acid sequence LEDYAFNFELILEEIARLFESG (SEQ ID NO:64) and the histidine of position 5, the histidine of position 8 and the tyrosine at position 11 of SEQ ID NO:61 and the asparagine at position 7 and the isoleucine at position 11 of SEQ ID NO:64 are not substituted.

In some embodiments, D1 comprises the amino acid sequence: KIQLHAEHALYDALMILNI (SEQ ID NO:62) and D3 comprises the amino acid sequence LEDYAFX₄₀FELX₄₄LEEIARLFESG (SEQ ID NO:65). In some such embodiments, at least one or both of X₄₀ and X₄₄ are serine. In some embodiments, the histidine at position 8 of SEQ ID NO: 62 is optionally substituted. The optional substituent can be, for example, phenylalanine. In some embodiments, the tyrosine at position 11 of SEQ ID NO:62 is optionally substituted. The optional substituent can be for example, lysine. In some embodiments, the histidine of position 5, the histidine of position 8 and the tyrosine at position 11 of SEQ ID NO:62 are not substituted. In some embodiments, the aspartic acid at position 12 of SEQ ID NO:62 is not substituted.

In some embodiments, D1 comprises the amino acid sequence set forth in SEQ ID NO: 61 and D3 comprises the amino acid sequence set forth in SEQ ID NO:64 and the Neo-2/15 mutant does not comprise a substitution at the glutamine at position 3, the leucine at position 4, the glutamic acid at position 7, the leucine at position 10, and the methionine at position 15 of SEQ ID NO:61 and the aspartic acid at position 3, the tyrosine at position 4, the phenylalanine at position 6, the leucine at position 10, and the glutamic acid at position 14 of SEQ ID NO:64. In some embodiments, the Neo-2/15 mutant also does not comprise a substitution at the leucine at position 14 of SEQ ID NO:61.

In some embodiments, D1 comprises the amino acid sequence set forth in SEQ ID NO: 62 and D3 comprises the amino acid sequence set forth in SEQ ID NO:65 and the polypeptide does not comprise a substitution at the glutamine of position 3, the leucine at position 4, the glutamic acid at position 7, the leucine at position 10, and the methionine at position 15 of SEQ ID NO:62 and the aspartic acid at position 3, the tyrosine at position 4, the phenylalanine at position 6, the leucine at position 10, and the glutamic acid at position 14 of SEQ ID NO:65. In some embodiments, the Neo-2/15 mutant also does not comprise a substitution at the leucine at position 14 of SEQ ID NO:62.

Included in the present invention are Neo-2/15 mutants wherein the glutamic acid at position 5, the glutamic acid at position 6, the asparagine at position 9, the isoleucine at position 12, the threonine at position 13, the serine at position 17, the isoleucine at 19 and the phenylalanine at position 20 of SEQ ID NO: 66 are not substituted. Included in the present invention are Neo-2/15 mutants wherein the leucine at position 14 of SEQ ID NO: 61 or 62 and at the phenylalanine at position 20 of SEQ ID NO:66 are not substituted. Included in the present invention are Neo-2/15 mutants wherein the leucine at position 14 of SEQ ID NO:61 or 62 and the isoleucine at position 12, the threonine at position 13, the serine at position 17, the isoleucine at position 19 and the phenylalanine at position 20 of SEQ ID NO: 66 are not substituted.

As used herein, a “position” in a SEQ ID NO refers to the sequential position in the amino acid sequence identified by the SEQ ID NO, including any X residues, unless indicated otherwise. For example, position 14 of SEQ ID NO:65, which has the sequence LEDYAFX₄₀FELX₄₄LEEIARLFESG is glutamic acid (underlined). Notwithstanding the foregoing, for certain amino acid sequences (such as SEQ ID NOs: 1, 3, 6, 14, 16, 20 and 81), the N-terminal amino acid may be designated as position 4. For those amino acid sequences where the N-terminal amino acid is designated as position 4, sequential numbering commences at 4. For example, position 95 of SEQ ID NO:3, which has the sequence KIQLHAEHALYSALMILNIVKINSPPAEEKLEDYAFNFELILEEIARLFESGDQKDEAEKAKRMKEW MKRIKTTASEDEQEEMANAIITILESWIFS, and wherein the first amino acid is designated as position 4, is glutamic acid (underlined). Sequential numbering starts at position 4 for certain sequences solely for position numbering consistency between sequences.

In any of these embodiments, D2 can comprise an amino acid sequence having at least at least 60%, at least 70%, at least 80%, at least 90%, or 100% identity to KDEAEKAKRMKEWMKRIKT (SEQ ID NO:63).

In any of these embodiments, the domain linkers can be, independently, 1-100, 1-90, 1-80, 1-70, 1-60, 1-50, 1-40, 1-30, 1-20, 1-10, or 2-10 amino acids in length.

In some embodiments, the order of the four domains is D1-D3-D2-D4, D4-D1-D3-D2, D2-D4-D1-D3, or D3-D2-D4-D1.

In some embodiments, the Neo-2/15 mutant comprises an amino acid sequence that is at least 90%, at least 91%, at least 92%, at least 93%, at least 94% at least 95%, at least 96% or at least 97% at least 98%, at least 99%, or 100% identical to an amino acid sequence selected from SEQ ID NO:3, 6, 14, 16, or 20. In some such aspects, D1 comprises three amino acids N-terminal and attached to the amino acid at position 1 of either SEQ ID NO:61 or 62. In some such aspects, the three amino acids are proline-lysine-lysine.

In some embodiments, the Neo-2/15 mutant comprises an amino acid sequence that is at least 90%, at least 95%, or 100% identical to an amino acid sequence selected from SEQ ID NOs: 2-25 or 75-82.

In some embodiments, the present invention provides a polypeptide comprising a Neo-2/15 mutant, wherein the Neo-2/15 mutant binds to IL-2Rβγ with an affinity that is at least 10 fold, or at least 100 fold, or at least 500 fold, or at least 1000 fold, or at least 10,000 fold attenuated as compared to Neo-2/15. In some embodiments, the Neo-2/15 mutant binds to IL-2Rβγ with an affinity that is at least 10 fold, or at least 100 fold attenuated as compared to Neo-2/15, but not more than 500 fold attenuated as compared to Neo-2/15. In some embodiments, the Neo-2/15 mutant binds to IL-2Rβγ with an affinity that is at least 100 fold or at least 500 fold attenuated as compared to Neo-2/15, but not more than 1000 fold attenuated as compared to Neo-2/15. In some embodiments, the Neo-2/15 mutant binds to IL-2Rβγ with an affinity that is at least 100 fold or at least 500 fold or at least 1000 attenuated as compared to Neo-2/15, but not more than 10,000 fold attenuated as compared to Neo-2/15.

In some embodiments, the present invention provides a polypeptide comprising a Neo-2/15 mutant, wherein the Neo-2/15 mutant binds to IL-2Rβγ with an affinity that is at least 10 fold, or at least 100 fold, or at least 500 fold, or at least 1000 fold, or at least 10,000 fold attenuated as compared to IL-2. In some embodiments, the Neo-2/15 mutant binds to IL-2Rβγ with an affinity that is at least 10 fold, or at least 100 fold attenuated as compared to IL-2, but not more than 500 fold attenuated as compared to IL-2. In some embodiments, the Neo-2/15 mutant binds to IL-2Rβγ with an affinity that is at least 100 fold or at least 500 fold attenuated as compared to IL-2, but not more than 1000 fold attenuated as compared to IL-2. In some embodiments, the Neo-2/15 mutant binds to IL-2Rβγ with an affinity that is at least 100 fold or at least 500 fold or at least 1000 attenuated as compared to IL-2, but not more than 10,000 fold attenuated as compared to IL-2.

In some embodiments, the present invention provides a polypeptide comprising a Neo-2/15 mutant, wherein the Neo-2/15 mutant binds IL-2Rβγ with an affinity that is within 2-fold, 3-fold, 4-fold, or 5-fold of the affinity of a Neo-2/15 mutant consisting of the sequence set forth in SEQ ID NO:4 for IL-2Rβγ. In some embodiments, the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, binds IL-2Rβγ with an affinity that is within 2-fold, 3-fold, 4-fold or 5-fold of the affinity of a reference polypeptide for IL-2Rβγ, wherein the reference polypeptide comprises a Neo-2/15 mutant fused to the C-terminus of an antibody, wherein the reference polypeptide comprises the amino acid sequences of SEQ ID NOs: 69 and 70.

In some embodiments, the present invention provides a polypeptide comprising a Neo-2/15 mutant, wherein the Neo-2/15 mutant stimulates STAT5 phosphorylation in Treg cells with an EC50 that is substantially the same or less than a Neo-2/15 mutant consisting of the sequence set forth in SEQ ID NO:4. In some embodiments, the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with an EC50 that is substantially the same or less than a reference polypeptide comprising a Neo-2/15 mutant fused to the C-terminus of an antibody, wherein the reference polypeptide comprises the amino acid sequences of SEQ ID NOs: 69 and 70.

In some embodiments, the present invention provides a polypeptide comprising a Neo-2/15 mutant, wherein the Neo-2/15 mutant stimulates STAT5 phosphorylation in Treg cells with a maximal signaling value that is substantially the same or greater than a Neo-2/15 mutant consisting of the sequence set forth in SEQ ID NO:4. In some embodiments, the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with a maximal signaling value that is substantially the same or greater than a reference polypeptide comprising a Neo-2/15 mutant fused to the C-terminus of an antibody, wherein the reference polypeptide comprises the amino acid sequences of SEQ ID NOs: 69 and 70.

In some embodiments, the present invention provides a polypeptide comprising a Neo-2/15 mutant, wherein the Neo-2/15 mutant stimulates STAT5 phosphorylation in Treg cells with a maximal signaling value that is at least 50%, at least 75%, at least 100% of the maximal signaling resulting from Neo-2/15 and/or IL-2 stimulation.

In some embodiments, the present invention provides a polypeptide comprising a Neo-2/15 mutant, wherein the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with an EC50 of 1 nM or less, 0.5 nM or less, or 0.1 nM or less and/or stimulates STAT5 phosphorylation in Teff cells with an EC50 of 1 nM or more.

In some embodiments, the present invention provides a polypeptide comprising a Neo-2/15 mutant, wherein the Neo-2/15 mutant stimulates NK cells with an EC50 that is substantially the same or higher than a Neo-2/15 mutant consisting of the sequence set forth in SEQ ID NO:4. In some embodiments, the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates NK cells with an EC50 that is substantially the same or higher than a reference polypeptide comprising a Neo-2/15 mutant fused to the C-terminus of an antibody, wherein the reference polypeptide comprises the amino acid sequences of SEQ ID NOs: 69 and 70.

The term “identity”, as used herein in reference to polypeptide sequences, refers to the subunit sequence identity between two molecules. When a subunit position in both molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained (including gaps if necessary). Identity may be calculated, in various embodiments, using published techniques and widely available computer programs, such as the GCG program package (Devereux et al., Nucleic Acids Res. 12:387, 1984), BLASTP, BLASTN, FASTA (Atschul et al., J. Molecular Biol. 215:403, 1990). Sequence identity can be measured, for example, using sequence analysis software such as the Sequence Analysis Software Package of the Genetics Computer Group at the University of Wisconsin Biotechnology Center (1710 University Avenue, Madison, Wis. 53705), using the default parameters. Unless indicated otherwise, percent identity is determined across the length of the reference sequence.

Substitutions, deletions, insertions or any combination thereof may be used to arrive at a final derivative or variant. Generally, these changes are done on a few amino acids to minimize the alteration of the molecule, particularly specificity of the antigen binding protein. However, larger changes may be tolerated in certain circumstances.

In some aspects, when it is desired to substitute an amino acid, but it is not desired for the amino acid substitution to significantly alter the profile of the protein, conservative amino acid substitutions are made. As used herein, “conservative amino acid substitution” means a given amino acid can be replaced by an amino acid having similar physiochemical characteristics, e.g., substituting acidic residues for another (such as E or D), substituting basic amino acids for another (such as K, R, or H), substituting the two amino acids having backbone distorted sidechains for another (G or P), substituting hydrophobic amino acids for another (L, I, V, A, or M), substituting aromatic hydrophobic amino acids for another (Y, F, or W), or substituting polar amino acids having uncharged side chains for another (T, S, N, Q, C). Polypeptides comprising conservative amino acid substitutions can be tested in any one of the assays described herein to confirm that a desired activity is retained. Amino acids can also be grouped according to similarities in the properties of their side chains. Alternatively, naturally occurring residues can be divided into groups based on common side-chain properties. Non-conservative substitutions will entail exchanging a member of one of these classes for a member of another class. Particular conservative substitutions include, for example, those set forth in Table 1.

TABLE 1

| Characteristic | Conservative Substitution |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

As used herein, the natural amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

### Treg Targeting Domains

The Neo-2/15 mutants of the present invention can be coupled to a targeting agent directed to Tregs, i.e., a Treg targeting agent, in order to provide them with optimal ability to preferentially expand and activate T-regulatory cells. Accordingly, the present invention provides a polypeptide comprising a Neo-2/15 mutant as described in any of the embodiments herein and a targeting agent. In particularly preferred embodiments, the targeting agent binds an antigen on the surface of Treg cells. Binding is preferably specific binding. Exemplary antigens are CD25 or CD39.

The Treg targeted agent can be any agent capable of directing the Neo-2/15 mutant to T-regulatory cells. In other words, the targeting agent can be any agent capable of directing the cellular localization of the Neo-2/15 mutant. The target of the targeting agent is typically a surface maker that is expressed on Tregs to a significantly higher degree than on any other cell types and is accessible to targeting.

In exemplary embodiments, the targeting agent is an antibody or antibody binding fragment of an antibody. The term “antibodies” specifically covers monoclonal antibodies, polyclonal antibodies, and bispecific antibodies. An antibody is a protein generated by the immune system that is capable of recognizing and binding to a specific antigen. The antibody can be of any type (e.g. IgG, IgE, IgM, IgD, and IgA), class (e.g. lgG1, lgG2, lgG3, lgG4, lgA1 and lgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2 m1, A2m2, Km1, Km2 and Km3) of immunoglobulin molecule. The subunit structures and three-dimensional configurations of different classes of immunoglobulins are well known. Antibodies may be murine, human, humanized, chimeric, or derived from other species such as rabbit, goat, sheep, horse or camel, but are preferably, human or humanized. The term “antibody,” as used herein, also includes antibody fragments capable of binding antigen, such as Fab, Fab′, and F(ab′)₂ fragments, etc.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20 (4): 450-459).

A full length antibody as used herein comprises an antigen binding domain fused directly to a constant region. For a conventional antibody, a full length antibody comprises two light chain polypeptides (each including a light chain variable region (VL) fused to a light chain constant region) and two heavy chain polypeptides (each including a heavy chain variable region (VH) fused to a heavy chain constant region). The heavy chain constant region comprises three domains, CH1, CH2, and CH3. Certain antibodies, such as camelid antibodies, comprise a single chain, typically a heavy chain, which comprises a variable region (or antigen-binding portion) and a constant region.

“Antigen-binding fragments” comprise a portion of a full length antibody capable of binding antigen, generally the antigen binding or variable region thereof, but may also comprise a constant region or a portion thereof. Examples of antibody fragments include Fab, Fab′, F(ab′)₂ fragments; scFv fragments; antigen binding portion of a single domain antibody, Fv fragments; minibodies; diabodies; triabodies; tetrabodies; linear antibodies; fragments produced by a Fab expression library, anti-idiotypic (anti-ld) antibodies, CDR (complementary determining region), and epitope-binding fragments of any of the above which immunospecifically bind to Treg cells.

The constant domain or an antibody can be involved in various effector functions, such as mediating antibody-dependent cellular cytotoxicity (ADCC), ADCP (antibody-dependent cellular phagocytosis), CDC (complement-dependent cytotoxicity) and complement fixation, binding to Fc receptors (e.g., CD16, CD32, FcRn), greater in vivo half-life, and protein A binding. In IgG, IgA, and IgD antibody isotypes, the Fc region is composed of two identical protein fragments derived from the second and third constant domains whereas IgM and IgE Fc regions contain three heavy chain constant domains.

As used herein, the term “Fc domain(s)” includes native and mutant forms Fc regions, including truncated forms. The Fc domain can provide extended serum half-life. Herein, specific residues within the Fc domain are identified by position according to the EU numbering scheme.

The IgG subclasses vary in their ability to mediate effector functions. For example, IgG1 is superior to IgG2 and IgG4 at mediating ADCC and CDC. Thus, in embodiments wherein effector function is undesirable, an IgG2 or IgG4 Fc region would be preferred. IgG2 Fc-containing molecules, however, are known to be more difficult to manufacture and have less attractive biophysical properties, such as a shorter half-life, as compared to IgG1 Fc-containing molecules. For that reason, IgG1 Fc-containing molecules with mutations to decrease effector function may be used instead of IgG2 Fc domains.

The effector function of an antibody can be increased, or decreased, by introducing one or more mutations into the Fc. In preferred embodiments of the invention, TRAs comprise an Fc domain engineered to decrease effector function. Exemplary Fc molecules having decreased effector function include those having one or more substitutions in an Fc domain corresponding to E233P, L234V, L234A, L235A, L235E, ΔG236, G237A, E318A, K320A, K322A, A327G, A330S, or P331S according to the EU index. In some exemplary embodiments, Fc domains having the following substitutions: N297A or N297Q (IgG1); L234A/L235A (IgG1); V234A/G237A (IgG2); L235A/G237A/E318A (IgG4); H268Q/V309L/A330S/A331S (IgG2); C220S/C226S/C229S/P238S (IgG1); C226S/C229S/E233P/L234V/L235A (IgG1); L234F/L235E/P331S (IgG1); S267E/L328F (IgG1). It is known that human IgG1 has a glycosylation site at N297 (EU numbering system) and glycosylation contributes to the effector function of IgG1 antibodies. An exemplary IgG1 sequence has a mutated N297 such as glutamine (N297Q) or with alanine (N297A) or with glycine (N297G). An Fc comprising a human IgG1 Fc having the N297G mutation may also comprise further insertions, deletions, and substitutions.

In some embodiments, an Fc domain that substantially lacks effector function has reduced binding affinity to Fcγ receptors of at least about 100-fold or at least about 1000-fold. In some embodiments, the Fc domain comprises what is known at the LALA mutations, a L234A and L235A mutation (EU numbering). In some embodiments, the Fc Region comprises a G237A, P329G or P329A mutation (EU numbering). In some embodiments, using the EU numbering system, the Fc domain comprises a L234A mutation, a L235A mutation, and/or a G237A mutation. Examples of amino acids that have been reported to reduce effector function include the LALA-PG mutations (L234A, L235A, P329G), LALA-PA mutations (L234A, L235A, P329A) or LALA-GA mutations (L234A, L235A, G237A). An exemplary Fc domain is set forth in SEQ ID NO:71. In certain embodiments the human IgG1 Fc comprises the LALA-PG or LALA-GA substitution and is at least 90% identical, at least 91% identical, at least 92% identical, at least 93% identical, at least 94% identical, at least 95% identical, at least 96% identical, at least 97% identical, at least 98% identical, at least 99% identical or 100% to the amino acid sequence set forth in SEQ ID NO:71. %

In some aspects, a polypeptide of the present invention comprises a full length antibody comprising a wild-type or mutant Fc domain. In other aspect, a polypeptide of the present invention comprises an antibody fragment such as a fusion of the variable regions of the heavy and light chains of an antibody (e.g., scFv). Polypeptides of the present invention comprising antibody fragments can also optionally comprise a heterologous polypeptide capable of increasing the circulating half-life of the Treg agonist in vivo. Such heterologous polypeptide can be, for example, a wild-type or mutant Fc domain.

Methods for the preparation of antibodies are known in the art. For example, antibodies can be prepared by immunizing a suitable mammalian host using an immunogenic protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989))

In addition, naked DNA immunization techniques known in the art are used (to generate an immune response to the encoded immunogen (for review, see Donnelly et al., 1997, Ann. Rev. Immunol. 15:617-648)

The amino acid sequence of a protein to which an antibody response is desired (e.g., CD25 for anti-CD25 antibodies) can be analyzed to select specific regions of the protein for generating antibodies. For example, hydrophobicity and hydrophilicity analyses of a protein's amino acid sequence are used to identify hydrophilic regions in the protein structure. Regions of a protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Hydrophilicity profiles can be generated using the method of Hopp, T. P. and Woods, K. R., 1981, Proc. Natl. Acad. Sci. U.S.A. 78:3824-3828. Hydropathicity profiles can be generated using the method of Kyte, J. and Doolittle, R. F., 1982, J. Mol. Biol. 157:105-132. Percent (%) Accessible Residues profiles can be generated using the method of Janin J., 1979, Nature 277:491-492. Average Flexibility profiles can be generated using the method of Bhaskaran R., Ponnuswamy P. K., 1988, Int. J. Pept. Protein Res. 32:242-255. Beta-turn profiles can be generated using the method of Deleage, G., Roux B., 1987, Protein Engineering 1:289-294. Thus, each region identified by any of these programs or methods is within the scope of the present invention. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances, linking reagents such as those supplied by Pierce Chemical Co., Rockford, Ill., are effective. Administration of an immunogen is often conducted by injection over a suitable time period and with use of a suitable adjuvant, as is understood in the art. During the immunization schedule, titers of antibodies can be taken to determine adequacy of antibody formation.

Monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay. When the appropriate immortalized cell culture is identified, the cells can be expanded and antibodies produced either from in vitro cultures or from ascites fluid.

The antibodies and fragments thereof can also be produced by recombinant means. Regions that bind specifically to the desired regions of a target can also be produced in the context of chimeric or complementarity-determining region (CDR) grafted antibodies of multiple species origin. Humanized or human antibodies can also be produced, and are preferred for use in therapeutic contexts. Methods for humanizing murine and other non-human antibodies, by substituting one or more of the non-human antibody CDRs for corresponding human antibody sequences, are well known (see for example, Jones et al., 1986, Nature 321:522-525; Riechmann et al., 1988, Nature 332:323-327; Verhoeyen et al., 1988, Science 239:1534-1536). See also, Carter et al., 1993, Proc. Natl. Acad. Sci. USA 89:4285 and Sims et al., 1993, J. Immunol. 151:2296.

Antibodies for use in the present invention can be fully human antibodies. Various methods in the art provide means for producing fully human monoclonal antibodies. For example, a preferred embodiment provides for techniques using transgenic mice, inactivated for antibody production, engineered with human heavy and light chains loci referred to as Xenomouse (Amgen Fremont, Inc.). Exemplary descriptions of preparing transgenic mice that produce human antibodies can be found, for example, in Mendez, et. Al. Nature Genetics, 15:146-156 (1998); Kellerman, S. A. & Green, L. L., Curr. Opin. Biotechnol 13, 593-597 (2002).

In addition, human antibodies of the invention can be generated using the HuMAb mouse (Medarex, Inc.) which contains human immunoglobulin gene miniloci that encode unrearranged human heavy (mu and gamma) and kappa light chain immunoglobulin sequences, together with targeted mutations that inactivate the endogenous mu and kappa chain loci (see e.g., Lonberg, et al. (1994) Nature 368 (6474): 856-859).

In another embodiment, fully human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, such mice are described in Tomizuka et al. (2000) Proc. Natl. Acad. Sci. USA 97:722-727 and PCT Publication WO 02/43478 to Tomizuka, et al.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In exemplary aspects, the targeting agent is an antibody or antigen binding fragments thereof that is directed to a surface maker expressed on Tregs to a significantly higher degree than on any other cell types and that is accessible to targeting. An exemplary Treg targeting agent is an antibody or antigen binding fragments thereof that binds CD25 (i.e, anti-CD25 antibody). Exemplary human CD25 polypeptide sequences are shown in Genbank accession no. NP_000408, version no. NP_000408.1, and Uniprot/Swiss-Prot accession No. P01589.

As used herein, “binds CD25” is used to mean the antibody binds CD25 with a higher affinity than the antibody binds an unrelated antigen, such as Bovine Serum Albumin. In some embodiments, the antibody binds CD25 with an association constant (Kₐ) at least 100, 200, 500, 1000, 2000, 5000, 104, 105 or 106-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, equal to or less than about 10⁻⁷ M, equal to or less than about 10⁻⁸ M, or equal to or less than about 10⁻⁹ M. Binding can be determined by ELISA, or flow cytometry, or surface plasmon resonance (SPR) technology, for example, in a BIAcore 3000 instrument using recombinant human IL-2Rα as the ligand and the antibody as the analyte.

Anti-CD25 antibodies or antigen binding fragments thereof suitable for use in the present invention can be blocking or non-blocking antibodies. Blocking antibodies are those that bind to CD25 and inhibit or block binding of IL-2 to CD25. For example, binding can be inhibited by at least about 10%, 20%, 30%, 40%, 50%, 60%, 70%, 80%, 90%, or 100%. Non-blocking antibodies do not inhibit or block binding of IL-2 to CD25.

Anti-CD25 antibodies or antigen binding fragments thereof are known in the art and may be used in the methods disclosed herein. Method of making anti-CD25 antibodies or antigen binding fragments thereof are also known in the art and may be used to make anti-CD25 antibodies or antigen binding fragments thereof for use in the present invention.

Anti-CD25 antibodies for use in the TRAs provided herein include, but are not limited to, the anti-CD25 antibodies described in U.S. Pat. No. 7,438,907, such as for example, antibodies AB1, AB7, Ab11, or AB12; anti-CD25 antibodies described in International Application No WO2020102591 such as D17, AH04526, AH04750, AH05285, AH05256, AH04527, AH05251, AH05285, AH05259, AH04750; anti-CD25 antibodies described in U.S. Pat. No. 6,383,487; and anti-CD25 antibodies described in U.S. Patent No. 20200010554, including 7D4, MA251 or 7G7B6.

Other anti-CD25 antibodies for use in the present invention include the chimeric antibody basiliximab and humanized version thereof, and anti-CD25 antibodies BT563 (see Baan et al., Transplant. Proc. 33:224-2246, 2001) and 7G8. An exemplary human antibody of use in the methods of the invention is HUMAX-TAC®, being developed by Genmab. HUMAX-TAC® is a human monoclonal antibody of the IgG1, kappa isotype, specific for human CD25 and having VH and VL domains with the sequences specified in SEQ ID NOs. 47 and 48 disclosed herein. Also useful in the present invention are antibodies 4C9 (obtainable from Ventana Medical Systems, Inc.) and antibody RFT5 (described in U.S. Pat. No. 6,383,487). Other suitable antibodies include B489 (143-13) (obtainable from Life Technologies, catalogue number MA1-91221), SP176 (obtainable from Novus, catalogue number NBP2-21755), 1B5D12 (obtainable from Novus, catalogue number NBP2-37349), 2R12 (obtainable from Novus, catalogue number NBP2-21755), BC96 (obtainable from BioLegend, catalogue number V T-072) and M-A251 (obtainable from BioLegend, catalogue number IV A053).

Additional anti-CD25 antibodies for use in the present invention include the PC61 antibody described in U.S. patent application No. 20080025947, the murine monoclonal antibody S4B6 and the monoclonal antibody MAB602, disclosed in Boyman et al., Science, (2006), 311:1924-1927.

In some embodiments the antibody is a humanized, deimmunized or resurfaced version of an antibody disclosed herein.

The anti-CD25 antibody daclizumab is an example of an Treg targeting agent that can be used in the present invention. The anti-CD25 antibody daclizumab is a humanized anti-CD25 antibody previously marketed under the trade name ZENAPAX. An exemplary anti-CD25 antibody is one comprising the daclizumab variable domains with a reduced/no effector function Fc domain.

The daclizumab heavy chain variable region (SEQ ID NO:26) comprises three heavy chain complementarity determining regions (CDRs), referred to herein (in amino- to carboxy-terminal order) as CDR-H1, CDR-H2 and CDR-H3, and designated SEQ ID NO:27 (CDR-H1); SEQ ID NO:28 (CDR-H2); and SEQ ID NO:29 (CDR-H3). The heavy chain framework (FR) sequences of daclizumab are designated SEQ ID NO:33 (FR-H1); SEQ ID NO: 34 (FR-H2); SEQ ID NO:35 (FR-H3); and SEQ ID NO:36 (FR-H4).

In some aspects, a polypeptide of the present invention comprises the heavy chain variable domain and light chain variable domain of the daclizumab antibody or mutated forms of the heavy and light chain variable domains of the daclizumab antibody (also referred to herein as daclizumab-related antibodies) or antigen binding fragments thereof. Such polypeptides may also comprise a wild-type FC domain, a variant FC domain, such as, for example, a FC domain with reduced effector function, a truncated FC domain (whether wild-type or variant) or no FC domain.

Daclizumab-related antibodies are known in the art, see for example, International Application No. WO2014144935 and U.S. Pat. No. 8,314,213 (incorporated by reference herein in their entirety). In some such embodiments, a polypeptide of the present invention comprises a daclizumab-related antibody, or antigen binding fragments thereof, that has reduced T cell immunogenicity as compared to daclizumab. In some embodiments, a polypeptide of the present invention comprises a daclizumab or daclizumab-related antibody, or antigen binding fragments thereof, characterized by one, two, three, or four of the following properties (i)-(iv):
(i) comprising the six CDRs set forth in NOs: 27, 28, 29, 30, 31 and 32;
(ii) comprising the six CDRs set forth in NOs: 27, 28, 29, 30, 31 and 32 except with up to 1, 2, 3, 4, 5, 6, 7, 8, 9, 10, 11, 12 13, 14, 15, 16 or 17 amino acid substitutions in the CDRs provided that any individual CDR has no more than 4 or no more than 3 amino acid substitutions as compared to the corresponding CDR sequence of an antibody having CDRs of SEQ ID NOs: 27, 28, 29, 30, 31 and 32, or any individual CDR other than CDR-H2 has no more than 3 or no more than 2 amino acid substitutions as compared to the corresponding CDR sequence of an antibody having CDRs of SEQ ID NOs: 27, 28, 29, 30, 31 and 32;
(iii) comprising the individual framework regions of SEQ ID NOs: 33, 34, 35, 36, 37, 38, 39 and 40 or framework regions with from 1-10, preferably 1-10 or 1-5 amino acid substitutions in such individual framework regions
(iv) comprising a VH and VL sequence having at least 75% sequence identity (and in certain embodiments, at least 80%, at least 85%, at least 90%, at least 95%, at least 98%, or at least 99% sequence identity) to the VH and VL sequences of daclizumab (SEQ ID NO: 26 and SEQ ID NO:41), and in some instances, comprising the CDRs of (i) or (ii).

In various embodiments, the daclizumab-related antibodies, or antigen binding fragments thereof, comprise one or more specific substitutions, including the amino acid substitution 148M in FR-H2 as compared to a FR-H2 of SEQ ID NO:34; the amino acid substitutions N52K and T54R in CDR-H2 as compared to CDR-H2 of SEQ ID NO:28 and S29K in CDR-L1 as compared to CDR-L1 of SEQ ID NO:30 and N53D in CDR-L2 as compared to CDR-L2 of SEQ ID NO:31; the amino acid substitutions N52K and T54R in CDR-H2 as compared to CDR-H2 of SEQ ID NO:28 and N53E in CDR-L2 as compared to CDR-L2 of SEQ ID NO:31; the amino acid substitutions N52S, S53R and T54K in CDR-H2 as compared to CDR-H2 of SEQ ID NO:28; the amino acid substitution T54S in CDR-H2 as compared to a CDR-H2 of SEQ ID NO:28; the amino acid substitutions S29K in CDR-L1 as compared to CDR-L1 of SEQ ID NO:30 and N53D in CDR-L2 as compared to CDR-L2 of SEQ ID NO:31; the amino acid substitutions S53R and T54K in CDR-H2 as compared to CDR-H2 of SEQ ID NO:28; the amino acid substitutions S29K in CDR-L1 as compared to CDR-L1 of SEQ ID NO:30 and N53D in CDR-L2 as compared to CDR-L2 of SEQ ID NO:31; and combinations thereof.

In various embodiments, the daclizumab-related antibodies, or antigen binding fragments thereof, comprise the amino acid substitutions (i) N52S, N52K, N52R or N52V and (ii) T54R, T54S or T54K in CDR-H2 as compared to CDR-H2 of SEQ ID NO:28, and, optionally, one or more of: (iii) S53R, S53K or S53N in CDR-H2 as compared to CDR-H2 of SEQ ID NO:28, (iv) Y56R in CDR-H2 as compared to CDR-H2 of SEQ ID NO:28, (v) E58Q in CDR-H2 as compared to CDR-H2 of SEQ ID NO: 28, (vi) E73K in CDR-H2 as compared to CDR-H2 of SEQ ID NO:28, (vii) S29K in CDR-L1 as compared to CDR-L1 of SEQ ID NO:30 and (viii) N53D or N53E in CDR-L2 as compared to CDR-L2 of SEQ ID NO: 31.

In various embodiments, the daclizumab-related antibodies, or antigen binding fragments thereof, comprise the amino acid substitutions (i) N52S, N52K, N52R or N52V, (ii) S53K, S53R or S53N and (iii) T54R, T54S or T54K in CDR-H2 as compared to CDR-H2 of SEQ ID NO: 28, (iv) S29K in CDR-L1 as compared to CDR-L1 of SEQ ID NO:30 and (v) N53D or N53E in CDR-L2 as compared to CDR-L2 of SEQ ID NO:31, and, optionally, further comprises the amino acid substitution (iv) Y56R in CDR-H2 as compared to CDR-H2 of SEQ ID NO:28.

In various embodiments, the daclizumab-related antibodies, or antigen binding fragments thereof, comprise the amino acid substitutions (i) N52S, (ii) S53R or S53K, (iii) T54S or T54K, and (iv) Y56R in CDR-H2 as compared to CDR-H2 of SEQ ID NO:28, (v) S29K in CDR-L1 as compared to CDR-L1 of SEQ ID NO:30 and (vi) N53D in CDR-L2 as compared to CDR-L2 of SEQ ID NO:31.

In various embodiments, the daclizumab-related antibodies, or antigen binding fragments thereof, comprise the amino acid substitutions (i) N52S, N52K, N52R or N52V and (ii) T54R, T54S or T54K in CDR-H2 as compared to CDR-H2 of SEQ ID NO:28, and, optionally, one or more of: (iii) S53R, S53K or S53N in CDR-H2 as compared to CDR-H2 of SEQ ID NO:28, (iv) Y56R in CDR-H2 as compared to CDR-H2 of SEQ ID NO:28, (v) E58Q in CDR-H2 as compared to CDR-H2 of SEQ ID NO:28, (vi) E73K in CDR-H2 as compared to CDR-H2 of SEQ ID NO:28, (vii) S29K in CDR-L1 as compared to CDR-L1 of SEQ ID NO:30 and (viii) N53D or N53E in CDR-L2 as compared to CDR-L2 of SEQ ID NO: 31. In some such embodiments, the framework regions have up to 4 amino acid substitutions as compared to frameworks of SEQ ID NO:3 (FR-H1), SEQ ID NO:5 (FR-H2), SEQ ID NO:7 (FR-H3), SEQ ID NO:9 (FR-H4), SEQ ID NO: 10 (FR-L1), SEQ ID NO: 12 (FR-L2), SEQ ID NO: 14 (FR-L3) and SEQ ID NO: 16 (FR-L4). In specific embodiments, the framework region comprises the amino acid substitution 148M in FR-H2 as compared to a FR-H2 of SEQ ID NO:5. In specific embodiments, the framework region does not comprise a substitution at 148 in FR-H2 as compared to a FR-H2 of SEQ ID NO:5.

In various embodiments, the daclizumab-related antibodies, or antigen binding fragments thereof, comprise the amino acid substitutions (i) N52S, N52K, N52R or N52V, (ii) S53K, S53R or S53N and (iii) T54R, T54S or T54K in CDR-H2 as compared to CDR-H2 of SEQ ID NO:28, (iv) S29K in CDR-L1 as compared to CDR-L1 of SEQ ID NO:30 and (v) N53D or N53E in CDR-L2 as compared to CDR-L2 of SEQ ID NO:31, and, optionally, further comprises the amino acid substitution (vi) Y56R in CDR-H2 as compared to CDR-H2 of SEQ ID NO:28. In some such embodiments, the framework regions have up to 4 amino acid substitutions as compared to frameworks of SEQ ID NO:33 (FR-H1), SEQ ID NO:34 (FR-H2), SEQ ID NO:35 (FR-H3), SEQ ID NO:36 (FR-H4), SEQ ID NO: 37 (FR-L1), SEQ ID NO:38 (FR-L2), SEQ ID NO:39 (FR-L3) and SEQ ID NO:40 (FR-L4). In specific embodiments, the framework region comprises the amino acid substitution 148M in FR-H2 as compared to a FR-H2 of SEQ ID NO:34. In specific embodiments, the framework region does not comprise a substitution at 148 in FR-H2 as compared to a FR-H2 of SEQ ID NO:34. In various embodiments, the anti-CD25 antibody or anti-CD25 binding fragment comprises the heavy and light chain variable domains of daclizumab with the substitutions N52S, S53R, T54K, in the heavy chain variable domain and the substitution N53E in the light chain variable domain; or with the substitutions N52K, T54R, in the heavy chain variable domain and the substitution N53E in the light chain variable domain. Numbering of the heavy chain and light chain variable regions is via Kabat numbering (see Tables 1 and 2 from WO2014/144935)

In various embodiments, the daclizumab-related antibodies, or antigen binding fragments, thereof have a heavy chain CDR1 having an amino acid sequence selected from the group consisting of SEQ ID NO:42, SEQ ID NO:43 and SEQ ID NO:27: a CDR2 having the amino acid sequence of SEQ ID NO:44; and a CDR3 having the amino acid sequence of SEQ ID NO:29; and a light chain comprising a CDR1 having the amino acid sequence of SEQ ID NO:45; a CDR2 having the amino acid sequence of SEQ ID NO:46; and a CDR3 having the amino acid sequence of SEQ ID NO:32. (See for example, U.S. Pat. No. 8,314,213, incorporated herein in its entirety and for all purposes.)

In some embodiments, a polypeptide of the present invention comprises an antibody or antigen-binding fragment thereof comprising a CDR-H1 comprising the amino acid sequence of SEQ ID NO: 27, 42, or 43; a CDR-H2 comprising the amino acid sequence of SEQ ID NO: 28 or 44; a CDR-H3 comprising the amino acid sequence of SEQ ID NO: 29; a CDR-L1 comprising the amino acid sequence of SEQ ID NO: 30 or 45; a CDR-L2 comprising the amino acid sequence of SEQ ID NO: 31 or 46; and a CDR-L3 comprising the amino acid sequence of SEQ ID NO: 32. In some such embodiments, the antibody or antigen-binding fragment thereof comprises a heavy chain variable region comprising the amino acid sequence of SEQ ID NO: 26 and a light chain variable region comprising the amino acid sequence of SEQ ID NO: 41.

In addition to or alternative to simply binding CD25, anti-CD25 antibodies, or antigen binding fragments thereof, may be selected for their retention of other functional properties, such as high affinity binding to CD25; and/or inhibition or blocking of CD25 binding to IL-2. In some embodiments, the anti-CD25 antibody, or antigen binding fragments thereof, to be selected will be one that, administered on its own, does not induce elimination of T cells expressing CD25 or inhibit the proliferation of T cells expressing CD25. In some aspects, the anti-CD25 antibody, or antigen binding fragments thereof, will bind to CD25 with the same or greater affinity as does daclizumab.

### Exemplary TRA Configurations

In some aspects, a TRA of the present invention comprises a Neo-2/15 mutant linked (e.g., via translational fusion or chemical/enzymatic conjugation) to an antibody or antigen binding fragment thereof. The TRA can optionally comprise amino acid linkers between one or more of the components (e.g., between Neo-2/15 mutant and antibody or antibody binding fragment) that make up the TRA. Non-limiting examples of linkers that connect different components of the TRA are glycine/serine linkers. For example, a glycine/serine linker can comprise a sequence of GGGGSGGGGSGGGGSGGGGS (SEQ ID NO: 49) or comprise a sequence of GGGGSGGGGSGGGGS (SEQ ID NO:50). This is simply a non-limiting example and the linker can have varying number of GGGGS (SEQ ID NO: 51) repeats. In some embodiments, the linker comprises 1, 2, 3, 4, 5, 6, 7, 8, 9, or 10 of the GGGGS (SEQ ID NO:51) repeats. In some embodiments, the linker comprises multiple glycine repeats such as GGGGGG (SEQ ID NO:52) or GGGGGGGG (SEQ ID NO:53). The linker to be used to connect different components of the TRA can be a flexible linker or a rigid linker. Non-limiting examples of rigid linkers are EAAK (SEQ ID NO:54); (EAAK)₂ (SEQ ID NO:55): (EAAK)₃ (SEQ ID NO:56): (EAAAK)₃ (SEQ ID NO:57): A(EAAAK)₄ALEA(EAAAK)₄A (SEQ ID NO:58); AEAAAKEAAAKA (SEQ ID NO:59); PAPAP (SEQ ID NO:60); and (ALA-PRO)₁₀₋₃₄ (SEQ ID NO: 83).

In some aspects, a polypeptide of the present invention comprises an antibody or antigen binding fragment of an antibody that targets a surface marker that is expressed on Tregs to a significantly higher degree than on any other cell types and is accessible to targeting wherein Neo-2/15 mutant is linked to either the C terminus or the N terminus of the antibody, optionally via an amino acid linker. In some aspects, a polypeptide of the present invention comprises a Neo-2/15 mutant and a scFv, wherein the Neo-2/15 mutant and scFv are both placed on opposite termini of the Fc. In some such aspects, the polypeptide comprises a homodimer comprising two Neo-2/15 mutants, each linked via their C terminus to the N terminus of a Fc domain which is linked via its C terminus to the variable heavy chain of a scFv. In some aspects, a polypeptide of the present invention comprises a Neo-2/15 mutant and an antibody, wherein the Neo-2/15 mutant is linked to the N terminus of a heavy chain of an antibody. In some such aspects, the polypeptide comprises a heterotetramer comprising two heavy chains (VH+constant region) and two light chains (VL+constant region), each heavy chain variable region attached via its N terminus to the C terminus of a Neo-2/15 mutant. In some aspects, a polypeptide of the present invention comprises a Neo-2/15 mutant and an antibody, wherein the Neo-2/15 mutant is linked to the C-terminus of the heavy chain of the antibody. In some such aspects, the polypeptide comprises a heterotetramer comprising two heavy chains (VH+constant region) and two light chains (VL+constant region), each heavy chain constant region attached via its C terminus to the N terminus of a Neo-2/15 mutant. Attachment can be, for example, via a linker. The linker can be, for example, a (GGGGS) n linker wherein n is 1-10.

In some embodiments, the polypeptides of the present invention comprise a Neo-2/15 mutant; a Neo-2/15 mutant linked to an anti-CD25 antibody as described herein; a Neo-2/15 mutant linked to an antigen binding fragment of an anti-CD25 antibody as described herein; or a Neo-2/15 mutant linked to an antigen binding fragment of an anti-CD25 antibody as described herein and a Fc domain as described herein. In some embodiments, the polypeptides of the present invention consist of or consist essentially of a Neo-2/15 mutant; a Neo-2/15 mutant linked to an anti-CD25 antibody as described herein; a Neo-2/15 mutant linked to an antigen binding fragment of an anti-CD25 antibody as described herein; or a Neo-2/15 mutant linked to an antigen binding fragment of an anti-CD25 antibody as described herein and a Fc domain as described herein. In some embodiments, a polypeptide of the present invention comprises the amino acid sequence of SEQ ID NO: 69 and the amino acid sequence of SEQ ID NO: 70. In some embodiments, a polypeptide of the present invention comprises the amino acid sequence of SEQ ID NO: 74 and the amino acid sequence of SEQ ID NO: 70. In some embodiments, a polypeptide of the present invention comprises a Neo-2/15 mutant linked to the C-terminus of the targeting agent or Fc domain, wherein the polypeptide comprises the amino acid sequence of SEQ ID NO: 69 and the amino acid sequence of SEQ ID NO: 70. In some embodiments, a polypeptide of the present invention comprises a Neo-2/15 mutant linked to the N-terminus of the targeting agent or Fc domain, wherein the polypeptide comprises the amino acid sequence of SEQ ID NO: 74 and the amino acid sequence of SEQ ID NO: 70.

In some embodiments, a polypeptide of the present invention does not substantially activate Teff cells, or the polypeptide activates Teff cells with an EC50 that is more than 5-fold, more than 10-fold, more than 50-fold, more than 100-fold, more than 500-fold, or more than 1000-fold higher than the EC50 for activating Treg cells.

In some embodiments, a polypeptide of the present invention does not substantially induce proliferation of Teff cells, or the polypeptide induces proliferation of Teff cells with an EC50 that is more than 5-fold, more than 10-fold, more than 50-fold, more than 100-fold, more than 500-fold, or more than 1000-fold higher than the EC50 for inducing proliferation of Treg cells.

In some embodiments, the maximal signaling by a polypeptide of the present invention in non-Treg cells is less than 30% or less than 20% of the maximal signaling of the non-Tregs by IL-2 (e.g., at a concentration of 10 nM or less of the polypeptide). In some embodiments, the non-Treg cells are Teff cells. In some embodiments, the non-Treg cells are NK cells.

In some embodiments, the maximal signaling by a polypeptide of the present invention in Tregs is at least 50%, at least 60%, or at least 70% of the maximal signaling in Tregs by IL-2 (e.g., at a concentration of 10 nM or less of the polypeptide).

In various embodiments, signaling in Tregs and non-Tregs is measured as % pSTAT5+ compared to IL-2. The EC50 may be determined from a % pSTAT5+ curve obtained using a titration of the test article.

In some embodiments, a polypeptide of the present invention induces STAT5 phosphorylation in Treg cells with an EC50 below 1 nM, below 500 pM, or below 100 pM and/or the polypeptide induces STAT5 phosphorylation in Teff cells with an EC50 above 1000 pM (i.e., 1 nM).

In some embodiments, a polypeptide of the present invention does not substantially activate or stimulate NK cells, or the polypeptide activates NK cells with an EC50 that is more than 5-fold, more than 10-fold, more than 50-fold, more than 100-fold, more than 500-fold, or more than 1000-fold higher than the EC50 for activating Treg cells. In some embodiments, the polypeptide does not substantially induce proliferation of NK cells, or the polypeptide induces proliferation of NK cells with an EC50 that is more than 5-fold, more than 10-fold, more than 50-fold, more than 100-fold, more than 500-fold, or more than 1000-fold higher than the EC50 for inducing proliferation of Treg cells. In some embodiments, the polypeptide induces STAT5 phosphorylation in Treg cells with an EC50 below 1 nM, below 500 pM, or below 100 pM and/or the polypeptide induces STAT5 phosphorylation in NK cells with an EC50 above 1000 pM.

### Nucleic Acids

Provided herein are nucleic acids comprising a nucleotide sequence encoding a polypeptide of the present invention, whether it be, for example, a Neo-2/15 mutant or a Neo-2/15 mutant fused to an antibody, a Neo-2/15 mutant fused to an antigen binding fragment of an antibody, a Neo-2/15 mutant fused to an antigen binding fragment of an antibody and a Fc domain, or another polypeptide of the present invention. The antibody or antibody fragment thereof may be, for example, an anti-CD25 antibody or antibody fragment thereof or an anti-CD39 antibody or antibody binding fragment thereof or another antibody capable of targeting Treg cells.

In some aspects, a nucleic acid of the present invention will encode a polypeptide comprising any one or more of sequences 1-82.

The nucleic acids and polypeptides of the present invention can be produced using any suitable method known in the art.

In exemplary aspects, the nucleic acid molecule comprises a nucleotide sequence encoding a polypeptide of the present disclosure. By “nucleic acid” as used herein includes %“polynucleotide”, “oligonucleotide”, and “nucleic acid molecule”, and generally means a polymer of DNA or RNA, or modified forms thereof, which can be single-stranded or double-stranded. The nucleic acid can comprise any nucleotide sequence which encodes any of the antigen-binding proteins or polypeptides of the present disclosure.

In some aspects, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.%

The nucleic acids in some aspects are constructed based on chemical synthesis and/or enzymatic ligation reactions using procedures known in the art. The nucleic acids of the present disclosure in some aspects are incorporated into a vector. In this regard, the present disclosure provides vectors comprising any of the presently disclosed nucleic acids. In exemplary aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell.

The vector of the present disclosure can be any suitable vector and can be used to transform or transfect any suitable host. Suitable vectors include those designed for propagation and expansion or for expression or both, such as plasmids and viruses. The vectors of the present disclosure can be prepared using standard recombinant DNA techniques. Constructs of expression vectors, which are circular or linear, can be prepared to contain a replication system functional in a prokaryotic or eukaryotic host cell. Replication systems can be derived, e.g., from CoIEl, 2μ plasmid, λ, SV40, bovine papilloma virus, and the like.% In some aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. The vector can include one or more marker genes, which allow for selection of transformed or transfected hosts. The vector can comprise a native or normative promoter operably linked to the nucleotide sequence encoding the polypeptide (including functional portions and functional variants thereof), or to the nucleotide sequence which is complementary to or which hybridizes to the nucleotide sequence encoding the TRA conjugate or fusion protein. The selection of promoters, e.g., strong, weak, inducible, tissue-specific and developmental-specific, is within the ordinary skill of the artisan. Similarly, the combining of a nucleotide sequence with a promoter is also within the skill of the artisan.%

Provided herein are host cells comprising a nucleic acid or vector of the present disclosure. As used herein, the term “host cell” refers to any type of cell that can contain the presently disclosed vector and is capable of producing an expression product encoded by the nucleic acid (e.g., mRNA, protein). The host cell in some aspects is an adherent cell or a suspended cell, i.e., a cell that grows in suspension. The host cell in exemplary aspects is a cultured cell or a primary cell, i.e., isolated directly from an organism, e.g., a human. The host cell can be of any cell type, can originate from any type of tissue, and can be of any developmental stage. The host cell can be a prokaryotic cell, such as a bacterial cell (e.g., E. coli) or a eukaryotic cell, such as a yeast cell or a mammalian cell. Whereas the unfused attenuated IL-2/1L-15 mimics can be expressed in prokaryotic or eurkaryotic cells, the TRAs are preferably expressed in mammalian cells.

Methods of making a polypeptide of the present invention are provided herein. The method, in exemplary embodiments, comprises culturing a host cell of the present disclosure to express the polypeptide and harvesting the expressed polypeptide. The host cell can be any of the host cells described herein. In exemplary aspects, the host cell is selected from the group consisting of: CHO cells, NS0 cells, COS cells, VERO cells, and BHK cells. In exemplary aspects, the step of culturing a host cell comprises culturing the host cell in a growth medium to support the growth and expansion of the host cell. In exemplary aspects, the growth medium increases cell density, culture viability and productivity in a timely manner. In exemplary aspects, the growth medium comprises amino acids, vitamins, inorganic salts, glucose, and serum as a source of growth factors, hormones, and attachment factors. In exemplary aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several growth media are commercially available and are described in the art. See, e.g., Arora, “Cell Culture Media: A Review” MATER METHODS 3:175 (2013).

### Pharmaceutical Compositions

Pharmaceutical compositions comprising a polypeptide of the present disclosure are provided herein. In exemplary aspects, the composition comprises agents which enhance the chemico-physico features of the polypeptide, e.g., via stabilizing, for example, the TRA at certain temperatures (e.g., room temperature), increasing shelf life, reducing degradation, e.g., oxidation protease mediated degradation, increasing half-life of, for example, the TRA.

In exemplary aspects of the present disclosure, the composition additionally comprises a pharmaceutically acceptable carrier, diluents, or excipient. In some embodiments, the polypeptide is formulated into a pharmaceutical composition comprising the active agent, along with a pharmaceutically acceptable carrier, diluent, or excipient. In this regard, the present disclosure further provides pharmaceutical compositions comprising an active agent which pharmaceutical composition is intended for administration to a subject, e.g., a mammal (such as a human).

In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia (USP) for use in animals, including humans. The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington 's Pharmaceutical Sciences, Sixteenth Edition, E. W. Martin (Mack Publishing Co., Easton, Pa., 1980), which is incorporated by reference in its entirety.

It will be evident to those of ordinary skill in the art that the optimal dosage of the active ingredient(s) in the pharmaceutical composition will depend on a variety of factors. Relevant factors include, without limitation, the type of animal (e.g., human), the particular form of polypeptides of the present invention, the manner of administration, and the composition employed.

In order to treat disease, the polypeptides of the present invention are provided in a therapeutically effective amount. This refers to an amount of the polypeptide effective for treating the disease or having the desired effect, i.e., the amount that will elicit the biological or medical response of a cell, tissue, system, or animal, such as a human, that is being sought by the researcher, veterinarian, medical doctor or other treatment provider. The data obtained from the cell culture assays and animal studies can be used in formulating a range of dosage for use in humans. Dosage regimens can be adjusted by clinicians to provide the optimum desired response. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a polypeptide of the present invention can include a single treatment or can include a series of treatments. The subject can be a mammal, including a human.

### Methods of Use

Exemplary polypeptides of the present invention may be used to expand Tregs within a subject or sample. Provided herein are methods of increasing the ratio of Tregs to non-Tregs. In some embodiments, provided herein are methods of increasing the ratio of Tregs to Teff cells and/or the ratio of Tregs to NK cells. In some aspects, the method comprises contacting a population of T cells with an effective amount of a polypeptide of the present invention. In some aspects, the population of T cells is within the peripheral blood of a subject. The typical Treg frequency in human blood is 5-10% of total CD4+CD3+ T cells, however, in diseases treatable by the present methods may be lower or higher. In preferred embodiments, the percentage of Treg increases by at least 10%, at least 20%, at least 30%, at least 40%, at least 50%, at least 60%, at least 70%, at least 80%, at least 90%, or by at least 100%. In certain embodiments, a polypeptide of the present invention is administered to a subject and the ratio of regulatory Tregs to Teffs within peripheral blood of a subject increases.

Because exemplary Treg agonists of the present invention will preferentially expand Tregs over non Tregs, exemplary TRAs are useful for increasing the ratio of regulatory T cells (Tregs) to natural killer (NK) cells within the peripheral blood of a subject. The ratio may be measured, for example, by determining the ratio of Tregs to CD16+ and/or CD56+ lymphocytes that are CD19− and CD3−.

Methods of the present invention include methods of activating Treg cells and/or methods of inducing proliferation of Treg cells comprising the step of contacting the Treg cell with a polypeptide of the present invention.

It is contemplated that a polypeptide of the present invention may have a therapeutic effect on a disease or disorder within a patient without significantly expanding the ratio of Tregs to non-regulatory T cells or NK cells within the peripheral blood of the patient. The therapeutic effect may be due to localized activity of the polypeptide at the site of inflammation or autoimmunity.

Exemplary Treg agonists of the present invention can be used, for example, to treat diseases, disorders, or conditions that will benefit from an expansion of Tregs without a corresponding expansion of non-regulatory T cells or NK cells within the peripheral blood of the patient.

Exemplary Treg agonists of the present invention can be used, for example, to treat diseases, disorders, or conditions associated with Teff cell activity.

Exemplary Treg agonists of the present invention can be used, for example, to treat diseases, disorders, or conditions associated with B cell activity.

Diseases, disorders, or conditions that are particularly amenable to treatment with a TRA include, but are not limited to, inflammation, autoimmune disease, atopic diseases, paraneoplastic autoimmune diseases, cartilage inflammation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reiter's Syndrome, SEA Syndrome (Seronegativity, Enthesopathy, Arthropathy Syndrome), dermatomyositis, psoriatic arthritis, scleroderma, vasculitis, myolitis, polymyolitis, dermatomyolitis, polyarteritis nodossa, Wegener's granulomatosis, arteritis, ploymyalgia rheumatica, sarcoidosis, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple sclerosis (MS), asthma, COPD, rhinosinusitis, rhinosinusitis with polyps, eosinophilic esophogitis, eosinophilic bronchitis, Guillain-Barre disease, Type I diabetes mellitus, thyroiditis (e.g., Graves' disease), Addison's disease, Raynaud's phenomenon, autoimmune hepatitis, GVHD, transplantation rejection, kidney damage, hepatitis C-induced vasculitis, spontaneous loss of pregnancy, and the like.

In preferred embodiments, the autoimmune or inflammatory disorder is lupus, graft-versus-host disease, hepatitis C-induced vasculitis, Type I diabetes, multiple sclerosis, spontaneous loss of pregnancy, atopic diseases, and inflammatory bowel diseases.

In another embodiment, a patient having or at risk for developing an autoimmune or inflammatory disorder or having such a disorder is treated with a TRA of the present invention and the patient's response to the treatment is monitored. The patient's response that is monitored can be any detectable or measurable response of the patient to the treatment, or any combination of such responses.

In some exemplary embodiments, a polypeptide of the present invention will be administered in combination with one or more additional agents. The additional agent may be an additional therapeutic agent used to treat the disorder or disease or it may be an agent that is administered in order to reduce side effects associated with treatment with the polypeptide.

In some embodiments, a subject or patient to be treated by the present methods is a mammal. In some aspects, the subject or patient is human.

### Kits

The present disclosure additionally provides kits comprising a polypeptide of the present invention. The kit in exemplary aspects comprises a polypeptide in a container. In exemplary aspects, the polypeptide is provided in the kit as a unit dose. For purposes herein “unit dose” refers to a discrete amount dispersed in a suitable carrier. In exemplary aspects, the unit dose is the amount sufficient to provide a subject with a desired effect, e.g., treatment of any one of the conditions or disorders described herein. In exemplary aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the polypeptide is pre-packaged in a ready to use form. In exemplary aspects, the ready to use form is for a single use. In exemplary aspects, the kit comprises multiple single use, ready to use forms of the polypeptide. In some aspects, the kit further comprises other therapeutic or diagnostic agents or pharmaceutically acceptable carriers (e.g., solvents, buffers, diluents, etc.), including any of those described herein.

### EXAMPLES

In all of the examples that reference anti-CD25 antibodies, the anti-CD25 antibody comprised the daclizumab heavy chain and light chain variable region binding domains. Fc domains as well as full length antibodies were IgG1 with reduced effector function imparted by the LALA-GA mutation (L234A, L235A, and G237A, numbering according to EU system) and optionally included a K447S modification (numbered according to the EU system) to prevent potential clipping of the C-terminal fusion domain, unless otherwise noted.

In all examples with pSTAT5 data, the following pSTAT5 titration protocol was used: Peripheral blood mononuclear cells (PBMCs) were separated from heparinized human whole blood using Lymphoprep density gradient medium and SepMate tubes. Pan CD3+ T cells were isolated from PBMCs using StemCell EasySep Human T Cell Isolation Kit and plated in each well of a 96-well round bottom plate with X-VIVO 15 media. Serial dilutions of test articles were diluted in X-VIVO 15 media and added to each well, and cells were stimulated for 30 minutes at 37° C. Cells were fixed by addition of paraformaldehyde to 1.5% and incubated for 10 minutes at room temperature. Cells were permeabilized in ice-cold 100% methanol for 30 minutes at 4° C. Cells were then washed twice with FACS buffer (phosphate-buffered saline with 2% fetal bovine serum and 1 mM EDTA). Cells were stained using CD4, CD8, CD25, CD127, and pSTAT5 antibodies conjugated to fluorophores for 1 hour at 4° C. Cells were washed with FACS buffer and fixed again with 1.5% paraformaldehyde. To assess NK cell signaling, the same procedure was performed with PBMCs that have not undergone CD3+ T cell isolation, with the addition of a CD56 antibody during the staining step. Flow cytometry analysis of cells was performed on the Cytek Aurora and analyzed using OMIQ cytometry analysis platform. Percentage of pSTAT5+ cells in different cell populations was determined by gating on unstimulated cell controls. Dose-response curves of % pSTAT5+ cells were fitted to a logistic model and half-maximal effective concentration (EC50 values) were calculated using GraphPad Prism data analysis software. Dose response curves were performed in the range of 0.01 pM to 100 nM. IL-2 control curves were included to determine responsiveness of donor cells, measured by % pSTAT5+ gating. Titrations were repeated at least three times per candidate to confirm consistent performance. To compare two different test articles or test articles with Neo-2/15 and/or IL-2, testing should be performed using cells from the same blood draw. There is significant signaling variability in patient blood draws. Tregs are defined as those T cells that are CD4+, CD25+ and CD127 (low/−). The notation all T cells, includes all CD3+ cells, including CD8+, CD4+, and Tregs.

When selecting a particular fusion protein for use as a TRA, the following parameters should be considered. First, candidates will preferably have low maximal signaling in non-Treg cells, such as Teff cells and/or NK cells (i.e., less than 30%, less than 20%, or less than 10% of maximal signaling resulting from IL-2 stimulation, or even lower) at, for example, concentrations up to 10 nM. In some embodiments, lower signaling in non-Tregs correlates with higher Treg selectivity. Second, maximal signaling in Tregs should preferably be at least 40%, at least 50%, or at least 60% of maximal signaling resulting from IL-2 stimulation, with higher maximal Treg signaling indicating a more potent TRA. Third, EC50 of the Treg % pSTAT5+ curve should preferably be below 1 nM, preferably even lower, such as below 500 pM, or below about 100 pM, with a lower Treg EC50 also indicating a more potent TRA. In addition to these three parameters, it is helpful to consider the Treg pSTAT5 MFI (mean fluorescence intensity) when selecting TRAs. A higher MFI corresponds with more potent signaling and downstream events, such as cell proliferation and activation. In some embodiments, when evaluating Emax of non-Tregs, if a high Emax (e.g., more than 20% of maximal signaling resulting from IL-2 stimulation) is only achieved with a high EC50, the test article may still be considered to have a Treg selective profile; precise evaluation will rely on assessment of the entire pSTAT5 titration curve between cell subsets.

### Example 1—Tuning Neo-2/15 Potency to Obtain Attenuated Neo-2/15 Mutants that Retain Binding to IL-2RBG and can be Used as TRAs when Fused to Treg Targeting Agents

To identify residues that were implicated in binding interactions between huIL2Rβ and/or IL2Rβγ and Neo-2/15, PDB structure 6DG5 was inspected in PyMol to select Neo-2/15 residues within 5.5 angstroms of either receptor. 27 positions were identified and Neo-2/15 mutant protein samples with single alanine mutations at those positions were cloned using Gibson Assembly into pET28 vector, expressed in Lemo21 cells, and purified by NTA-Ni and size-exclusion chromatography. Neo-2/15 mutants with single alanine mutations at each receptor interface position, had binding affinities to huIL2Rβ, and huIL2Rβγ analyzed by Octet Biolayer interferometry. Binding data was collected in the Octet RED96 instrument and processed using the integrated Octet software. Binding to huIL2Rβ was collected using Human IL-2 R beta/CD122 Protein, Fc Tag (MALS & SPR verified) (ILB-H5253), and binding to huIL2Rβγ was collected using Human IL-2 R gamma/CD132 Protein, Fc Tag (ILG-H5256) with Human IL-2 R beta/CD122 Protein, His Tag (SPR verified) (CD2-H5221) at concentrations equimolar or greater to the analyte concentration. Anti-Human Fc Capture (AHC) sensors were equilibrated for 90s in 1× Cytiva HBS-EP+ Buffer 10 binding buffer. Sensors were loaded with receptor by mixing with wells containing 0.5 ug/ml aforementioned receptors until a threshold response reached 1.0 nm. Sensors were then baselined in binding buffer for 90s. After baseline measurement, the association kinetics (kon) were monitored by dipping the biosensors in wells containing analyte for 400s. Analyte concentrations decreased as 3-fold dilutions from 90, 30, 10, 3.3, 1.1, and 0.4 nM for huIL2Rβ and 600, 200, 66.6, 22.2, 7.4, 2.5 nM for huIL2Rβγ. Dissociation kinetics (koff) were monitored by dipping the biosensors in separate wells of binding buffer for 800s. The dissociation constant (Kd) was calculated using a 1:1 steady-state binding model with the Octet software.

After calculating the binding affinities for 27 positions to huIL2Rβ and huIL2Rβγ with single alanine mutation screening, four positions were identified for tuning. Each variant had folding and Tm characterized to confirm reduced affinity was not a result of misfolding, and SEC traces were tracked to confirm no aggregation in the samples tested. The 27 positions identified within 5.5 angstroms of either receptor were positions Q6, L7, H8, E10, H11, L13, Y14, D15, L17, M18, K33, D36, Y37, F39, N40, L43, 144, E47, E84, E85, N88, 191, T92, Q95, S96, 198, F99. Out of the 27 positions, 4 positions were identified for tuning in a first round of attenuation. Table 2 shows the Kds for Neo-2/15 mutants with the noted single alanine mutations.

TABLE 2

| Protein | IL-2RB Kd (nM) | IL-2RBG Kd (nM) |
|---|---|---|
| Neo-2/15 | 1.6 | 6 |
| Neo-2/15_D15A | 140 | 230 |
| Neo-2/15_N40A | 60 | 44 |
| Neo-2/15_I44A | 84 | 58 |
| Neo-2/15_Q95A | 3.0 | 190 |

To identify combinations of alanine mutations on Neo-2/15 to further attenuate its binding to huIL2Rβ and huIL2Rβγ, the 4 identified positions were combined in different possibilities. Low attenuation was measured as 1-10× reduced Kd relative to Neo-2/15; moderate attenuation was measured as 10-100× reduced Kd relative to Neo-2/15; high attenuation was measured as 100×+ reduced Kd relative to Neo-2/15 (typically between 100× and 1000× reduced Kd relative to Neo-2/15).

The mutation at position 95 was to attenuate binding to IL-2G whereas the mutations at position 15, 40, and 44 were to attenuate binding to IL-2B.

Attenuation to the gamma domain is difficult to measure on its own because Neo-2/15 binding to IL2RG alone is weak, in the low uM range, so affinity needs to be measured in the presence of IL2Rβ. Further, in embodiments, where the mutant has moderate to high attenuation, binding via octet is typically measured under high avidity conditions.

For high avidity conditions, the attenuated Neo-2/15 mutants are fused to a full length antibody. The antibody is a control antibody (i.e., it is not an anti-IL-2Rβ or anti-IL-2RG binding antibody). For the attenuated Neo-2/15 mutants, a reduced effector function anti-CD25 antibody was used and for the Neo-2/15 control, trastuzumab was used. Biotinylated Human IL-2Rb or heterodimeric biotinylated human IL-2Rb+IL-2Rg-Fc (Acro Biosystems, ILG-H82F3) was immobilized to Anti-Streptavidin sensors at 2 μg/mL in binding buffer (10 mM HEPES, pH 7.4, 150 mM NaCl, 3 mM EDTA, 0.05% surfactant P20, 0.5% non-fat dry milk). After baseline measurement in the binding buffer alone, the binding kinetics were monitored by dipping the biosensors in wells containing defined concentrations of Neo-2/15 mutant (333-3000 nM) to measure association and then dipping the sensors back into baseline wells to measure dissociation. Dissociation constants were calculated using a steady-state binding model with the Octet software. The results demonstrated that for the anti-CD25 fusion protein comprising the Neo-2/15 D15S_Q95E mutant, the fusion protein bound to IL-2RBG but with weaker affinity than Neo-2/15 or Neo-2/15 bound to trastuzumab.

### Example 2—Neo-2/15 Mutant Demonstrated Attenuated pSTAT5 Activity

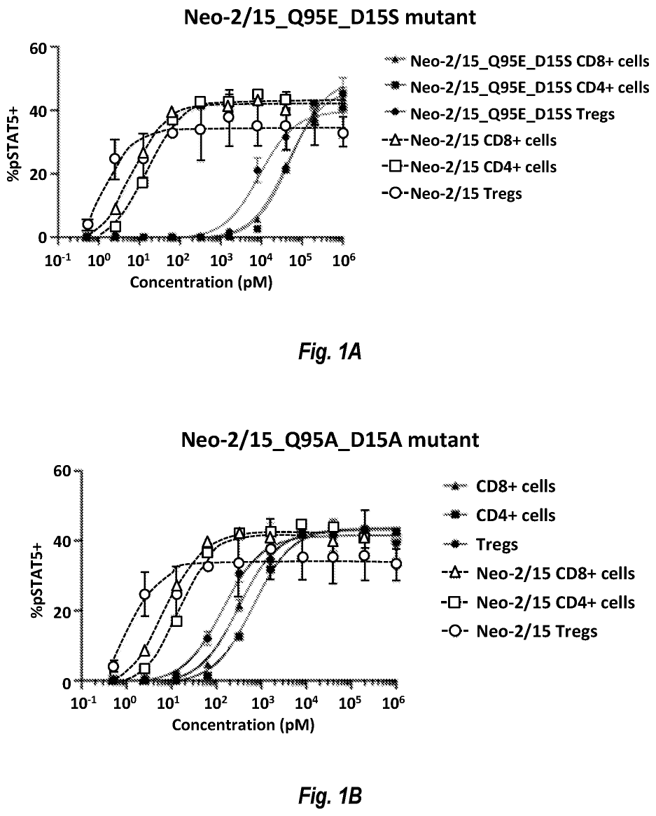

Neo-2/15 mutants are recombinantly expressed and purified from E coli using methods described in U.S. Pat. No. 10,703,791. A Neo-2/15 mutants having the Q95E and D15S mutations (Neo-2/15_D15S_Q95E) or the Q95A D15A (Neo-2/15_D15A_Q95A) mutations (Neo-2/15_D15A_Q95A) was tested for its ability to stimulate STAT5 phosphorylation in T cells in vitro. Both mutants demonstrated attenuated pSTAT5 activity on all cell types tested, CD8+ cell types, CD4+ cell types, and T-regs, with Neo-2/15_D15S_Q95E displaying more attenuation than Neo-2/15_D15A_Q95A. Notably, Neo-2/15_D15S_Q95E still retained a Treg EC50 of approximately 9 nM and Treg maximal pSTAT5 signaling of about 40%. See FIGS. 1A and 1B; Table 3. The higher level of attenuation of Neo-2/15_D15S_Q95E translated into better TRA activity (see following examples).

TABLE 3

| Payload | CD8+ | | CD4+ | | Treg | |
|---|---|---|---|---|---|---|
| only | EC50 in pM (Emax) | | | | | |
| Neo-2/15 | 6.4 | (41.8%) | 15.1 | (43%) | 0.9012 | (34%) |
| Neo-2/15_{—} | 42746 | (46%) | 52498 | (49.8%) | 9083 | (39.7%) |
| D15S_Q95E | | | | | | |
| Neo-2/15_{—} | 315.1 | (43%) | 663.6 | (43.6%) | 139.2 | (41.5%) |
| D15A_Q95A | | | | | | |

### Example 3—Neo-2/15 Mutants can be Fused in Different Configurations to Treg Targeting Agents and Fc Domains

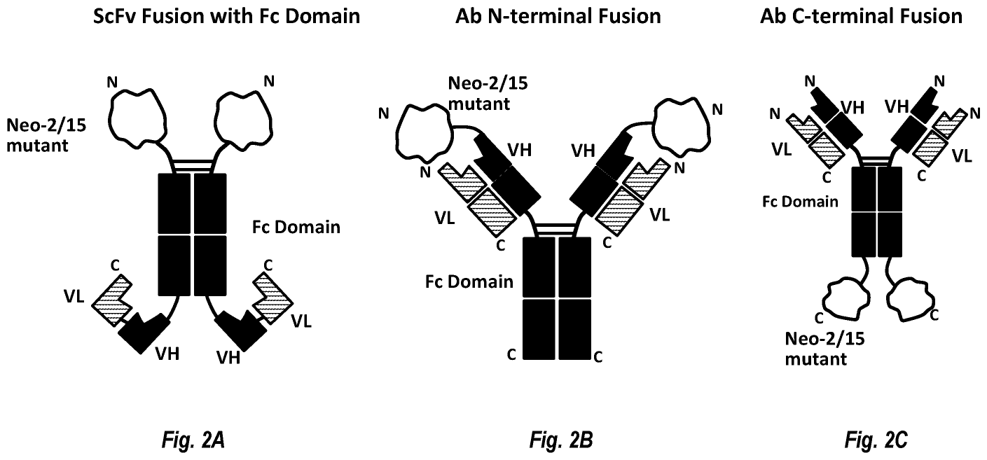

Neo-2/15 mutants were cloned into pcDNA3.4 in a variety of daclizumab binding domain fusion protein formats and expressed in Expi293 cells. The designs differed in terms of the format of the binding domain (scFv or full-length mAb with reduced/no effector function) as well as the site of the Neo-2/15 mutant and scFv fusion. Exemplary configurations are provided in FIGS. 2A-C.

In FIG. 2A, a Neo-2/15 mutant and scFv are both placed on opposite termini of the Fc. The TRA is a fusion protein comprising a homodimer comprising two Neo-2/15 mutants, each linked via their C terminus to the N terminus of a Fc domain which is linked via its C terminus to the variable heavy chain of a ScFv.

In FIG. 2B, a Neo-2/15 mutant is fused to the N terminus of a heavy chain variable region. The TRA is a fusion protein comprising a heterotetramer comprising two heavy chains (VH+constant region) and two light chains (VL+constant region), each heavy chain variable region attached via its N terminus to the C terminus of a Neo-2/15 mutant.

In FIG. 2C, a Neo-2/15 mutant is fused to the C-terminus of the heavy chain. The TRA is a fusion protein comprising a heterotetramer comprising two heavy chains (VH+constant region) and two light chains (VL+constant region), each heavy chain constant region attached via its C terminus to the N terminus of a Neo-2/15 mutant. The linker (GGGGSGGGGSGGGGS) was used.

### Example 4—Neo-2/15 Mutant-ScFv Fusion Proteins Demonstrated TRA Activity in a pSTAT5 Signaling Assay

Neo-2/15 mutants were fused to anti-CD25 ScFv antibody fragments (no Fc domains) and tested in a pSTAT5 assay to identify fusion proteins that are able to achieve a reduction of Teff signaling while maintaining Treg signaling.

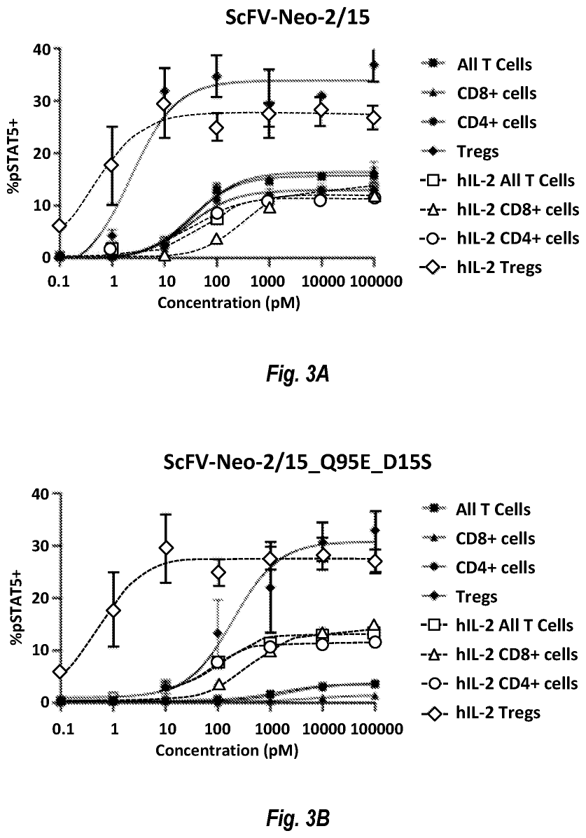

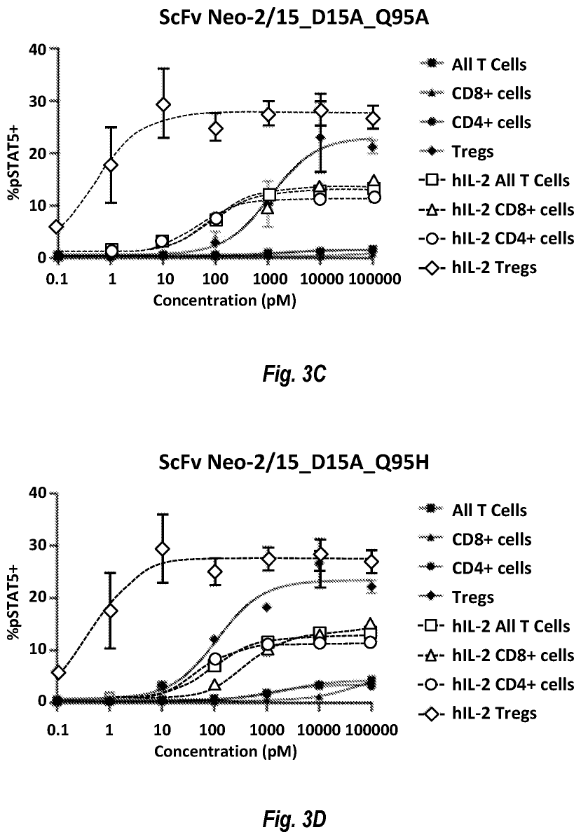

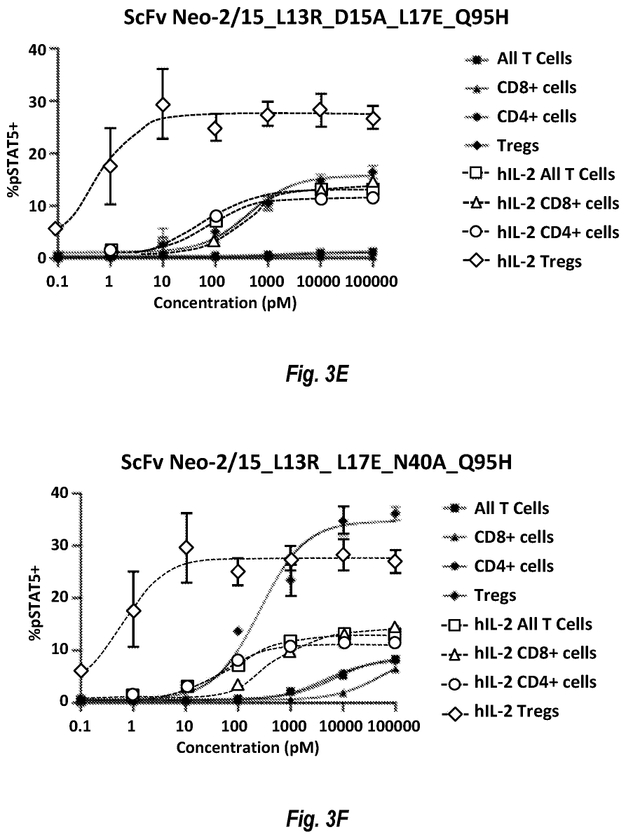

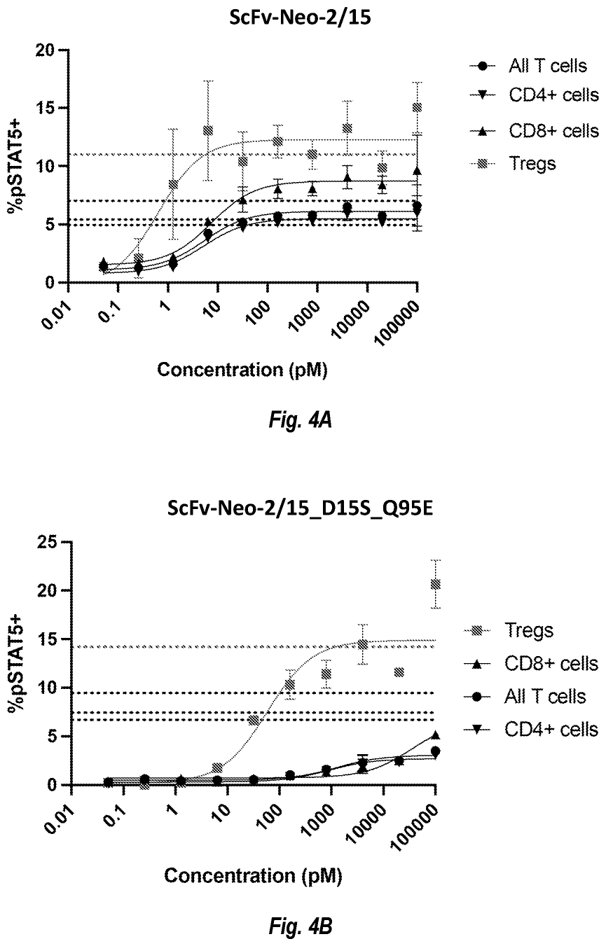

In a panel of ScFv fusion proteins shown in Table 4, Neo-2/15 mutant_D15S_Q95E fusion protein demonstrated the best reduction of Teff signaling while maintaining Treg signaling. See also FIGS. 3A-F. For FIGS. 4A-B, IL-2 control was added at 1 nM constant concentration, resulting in average % pSTAT5+ signaling of 7.44% for All T cells, 9.45% for CD8+ T cells, 6.71% for CD4+ non-Tregs, and 14.2% for Tregs, as represented by dotted lines.

TABLE 4

| ScFv Fusion | | | | |
|---|---|---|---|---|
| Proteins | All T cells | CD8+ | CD4+ | Treg |
| (no Fc domain) | Ec50 in pM (Emax) | | | |
| Neo-2/15 3A | 32.22 | 36.29 | 29.45 | 2.288 |
| | (15.71.1%) | (16.4%) | (12.97%) | (33.88%) |
| Neo-2/15_D15S_Q95E | 1475 | 6982 | 2253 | 197.8 |
| | (3.622) | (1.5%) | (3.7%) | (30.8%) |
| Neo-2/15_D15A_Q95A | 1910 | — | 2007 | 1128 |
| | (1.5%) | | (1.5%) | (23%) |
| Neo-2/15_D15A_Q95H | 2325 | 52177 | 962 | 113 |
| | (4.3%) | (5.9%) | (3.5%) | (23.4%) |
| Neo-2/15_L13R_{—} | 2167 | 178.5 | 2827 | 424.2 |
| D15A_L17E_Q95H | (1.2%) | (0.3%) | (1%) | (15.8%) |
| Neo-2/15_L13R_{—} | 7027 | 53623 | 4498 | 260.5 |
| L17E_N40A_Q95H | (8.8%) | (9.7%) | (8.35) | (34.7%) |
| hIL-2 unfused | 86.1 | 390 | 42 | 0.5627 |
| control | (12.9%) | (13.8%) | (11.3%) | (27.6) |

| ScFv Fusion | All T cells | CD8+ | CD4+ | Treg |
|---|---|---|---|---|
| Proteins | Ec50 in pM (Emax) | | | |
| Neo-2/15 | 5.417 | 7.463 | 5.244 | 0.6442 |
| | (6.1%) | (8.7%) | (5.5%) | (12.2%) |
| Neo- | 1253 | 35337 | 892.7 | 58.73 |
| 2/15_D15S_Q95E | (3%) | (6.7%) | (2.7%) | (14.9%) |
| Neo-2/15_N40A | 39.84 | 231.8 | 31.16 | 1.928 |
| | (5.7%) | (8.3%) | (5.2%) | (12.33%) |
| Neo- | 121.5 | 687.7 | 89.78 | 3.157 |
| 2/15_N40A_Q95E | (6.4%) | (9.8%) | (5.8%) | (15%) |
| Neo- | 599.2 | 3854 | 407.7 | 25.65 |
| 2/15_N40A_I44A | (5.5%) | (8.3%) | (4.9%) | (16.8%) |
| Neo-2/15_D15S | 427.6 | 1740 | 330.2 | 8.907 |
| | (4.8%) | (6.5%) | (4.4%) | (12.6%) |

### Example 5—Fusion Proteins of the Present Invention can be Optimized for Higher Affinity to CD25 than IL-2

Binding to human CD25 was determined by Octet. Unlike IL-2 and IL-2 mutant Treg agonist, the Neo-2/15 mutants of the present invention have no binding site for CD25. Affinity to CD25 can be modulated via selection of the anti-CD25 binding agent. TRAs comprising Neo-2/15 mutants fused to anti-CD25 antibody demonstrate higher affinity to CD25 than IL-2. A ScFv Neo-2/15_D15S_Q95E fusion protein (no Fc domain) demonstrated about 25 times higher binding than hIL-2 to human CD25 with a Kd of 0.5 nM as compared to 13 nM (data not shown).

### Example 6—Fusing Neo-2/15 Mutants to an Anti-CD25 Treg Targeting Agent Enhances Treg Selectivity

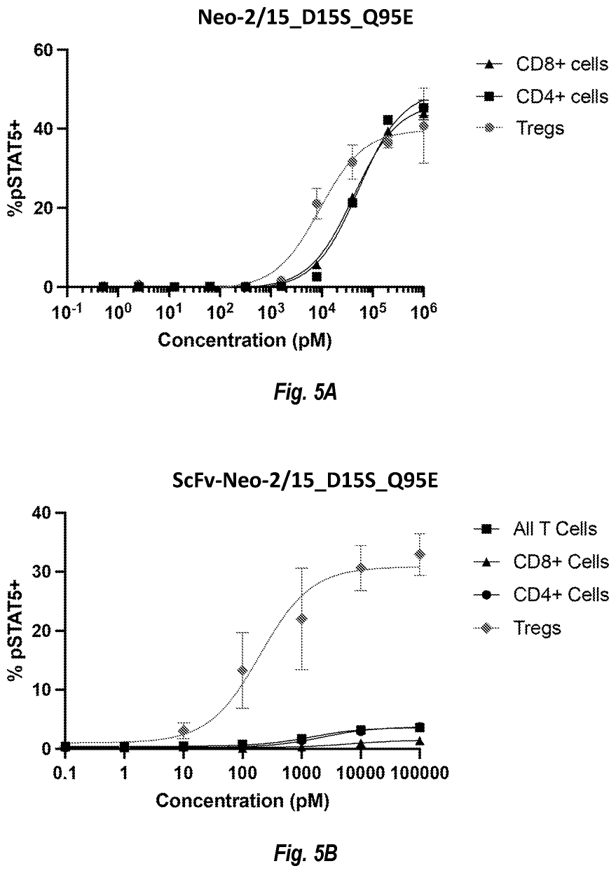

pSTAT signaling of Treg cells and Teff cells for the unfused Neo-2/15 Neo-2/15 mutant_D15S-Q95E, an anti-CD25 Neo-2/15 mutant fusion protein comprising Neo-2/15_D15S-Q95E, and IL-2 was evaluated. Fusing the Neo-2/15_D15S-Q95E mutant to the anti-CD25 ScFv enhanced potency on Treg cells while reducing potency on CD8+ and CD4+ T cells. The fusion protein demonstrated TRA activity. This was an unexpected result as it was not known that fusing a T-reg targeting agent to an attenuated Neo-2/15 mutant would cause a differential reduction in Teff signaling as compared to Treg signaling. See FIGS. 5A-B and Table 5.

TABLE 5

| | CD8+ | CD4+ | Treg |
|---|---|---|---|
| Test Article* | Ec50 in pM (% Emax) | | |
| Neo-2/15_D15S_Q95E | 47000 (47%) | 52000 (50%) | 9000 (40%) |
| (payload only) | | | |
| Neo_2/15_D15S_Q95E | 7000 (1%) | 2000 (4%) | 198 (31%) |
| ScFv fusion protein | | | |

*Note that these experiments are with different donors.

### Example 7—Fusion Proteins of the Present Invention Comprising Neo-2/15_D15S_Q95E Demonstrated Treg Selectivity as Measured in a pSTAT5 Assay in Different Antibody Configurations

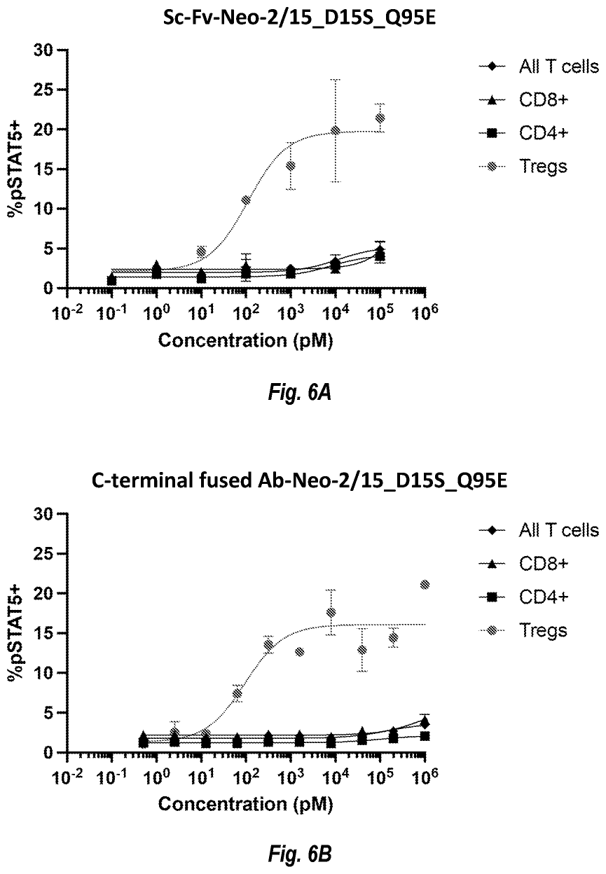

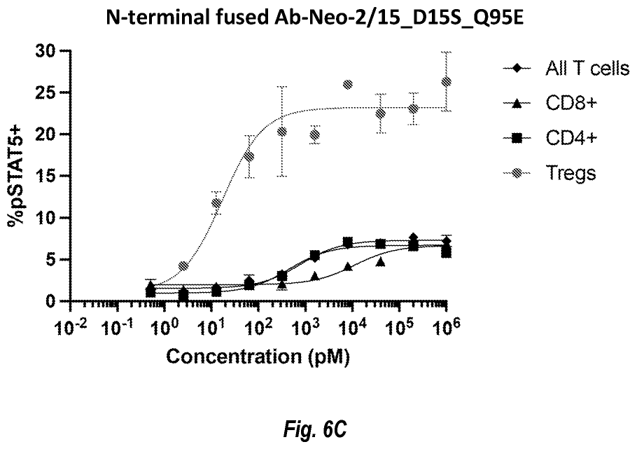

The Neo-2/15_D15S_Q95E mutant was fused to anti-CD25 antibody in different configurations as shown in FIG. 2. The fusion proteins that demonstrated the best TRA activity are shown in FIGS. 6A-C and Table 6. For the Neo-2/15_D15S_Q95E fusion protein, the mutant bound to the C terminal of a full length antibody is the preferred configuration

TABLE 6

| Neo- | CD8+ | | CD4+ | | Treg | |
|---|---|---|---|---|---|---|
| 2/15_D15S_Q95E | | | | | | |
| Fusions | Ec50 in pM (% Emax) | | | | | |
| ScFv fusion | — | | 8000 | (4%) | 112 | (20%) |
| Fusion to the C- | 778000 | (1%) | 101000 | (2%) | 95 | (16%) |
| terminus of heavy | | | | | | |
| chain of full length | | | | | | |
| anti-CD25 Ab | | | | | | |
| Fusion to the N- | 11000 | (7%) | 447 | (7%) | 17 | (23%) |
| terminus of heavy | | | | | | |
| chain variable | | | | | | |
| domain of full | | | | | | |
| length anti-CD25 | | | | | | |
| Ab | | | | | | |

### Example 8—Additional Tuning of Neo-2/15 Mutant Component of TRAs

Another panel of mutations were made in order to identify whether mutations at additional positions involved in binding to Il-2RB or IL-2RBG could be added to the Neo_2/15_D15S_Q95E mutant while still maintaining its pSTAT activity profile and whether other combinations could lead to alternative TRAs. The additional positions that were identified were positions 8, 11, and 14. Table 7 shows Kds of mutants with the one noted mutation from single alanine mutation scanning. The combinations of mutations that were made for testing were D15S+N40S; D15S+N40S+I44S; H8R+D15S; H8R+D15S+N40S+I44S; H8R+N40S+I44S; H11F+D15S; H11F+D15S+N40S+I44S; H11F+N40S+I44S; Y14K+D15S+N40S+I44S; Y14K+D15S; H11F+N40S+I44S; D15S+N40S+Q95E; D15S+N40S+I44S+Q95E; H8R+D15S+Q95E; H8R+D15S+N40S+I44S+Q95E; H8R+N40S+I44S+Q95E; H11F+D15S+Q95E; H11F+D15S+N40S+I44S+Q95E; H11F+N40S+I44S+Q95E; N40S+I44S+Q95E; N40S+Q95E; Y14K+D15S+Q95E; Y14K+D15S+N40S+I44S+Q95E; and Y14K+N40S+I44S+Q95E.

TABLE 7

| Protein | IL-2RB Kd (nM) | IL-2RBG Kd (nM) |
|---|---|---|
| Neo-2/15 | 1.6 | 6 |
| Neo-2/15_H8A | 1.4 | 11 |
| Neo-2/15_H11A | 3.3 | 15 |
| Neo-2/15_Y14A | 0.7 | 10-20* |

Fusion proteins were made with each mutant expressed as C- or N-terminal fusions to the full length anti-CD25 antibody in order to determine whether the position of fusion had an effect on activity. When fused to the C terminus, the Neo-2/15 mutant is distal from the IL-2A receptors, whereas when fused at the N terminus, the Neo-2/15 mutant is proximal to the receptors. Certain fusion were effective as TRAs when fused to one terminal, but not as effective at the other. Generally, when fused at the N-terminus, the fusion proteins demonstrated higher Teff activity than at the C-terminus.

When fused to the C terminus, if the D15S and Q95E mutations were present, all additional mutations made had the effect of reducing the Treg STAT5 phosphorylation activity to an undesirable level. Without being bound by theory, it is believed that the level of attenuation for the D15S_Q95E mutant is at an optimal level for C-terminal fusion and any additional mutations made to this mutant are preferably made at positions not involved in binding to IL-2Rβ or IL-2RBG provided that the intent is a C terminal fusion to a full length antibody.

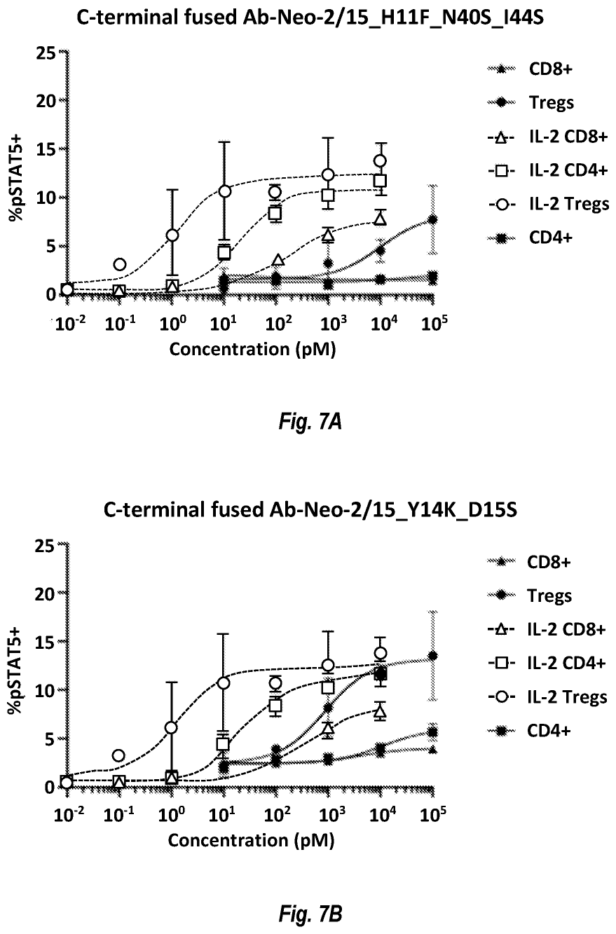

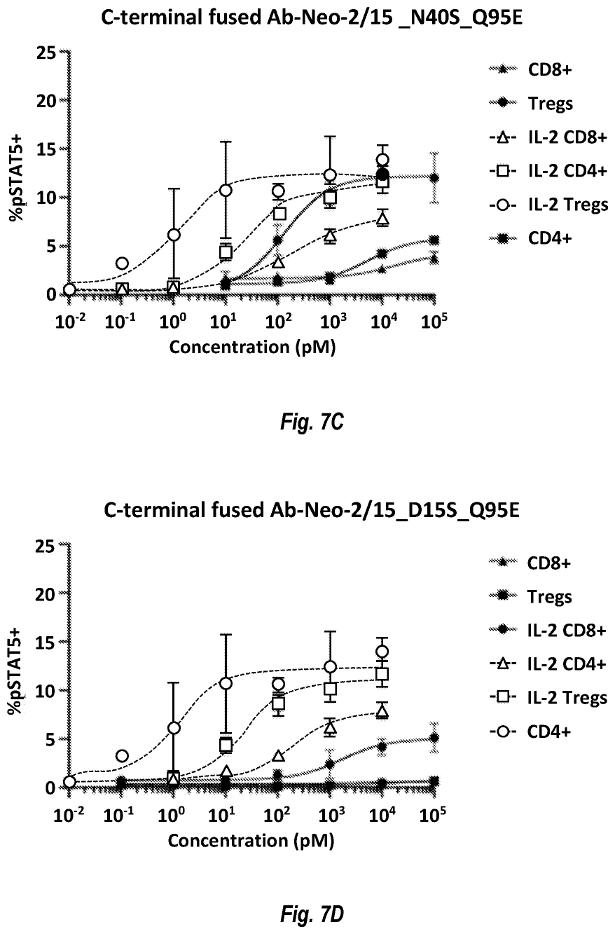

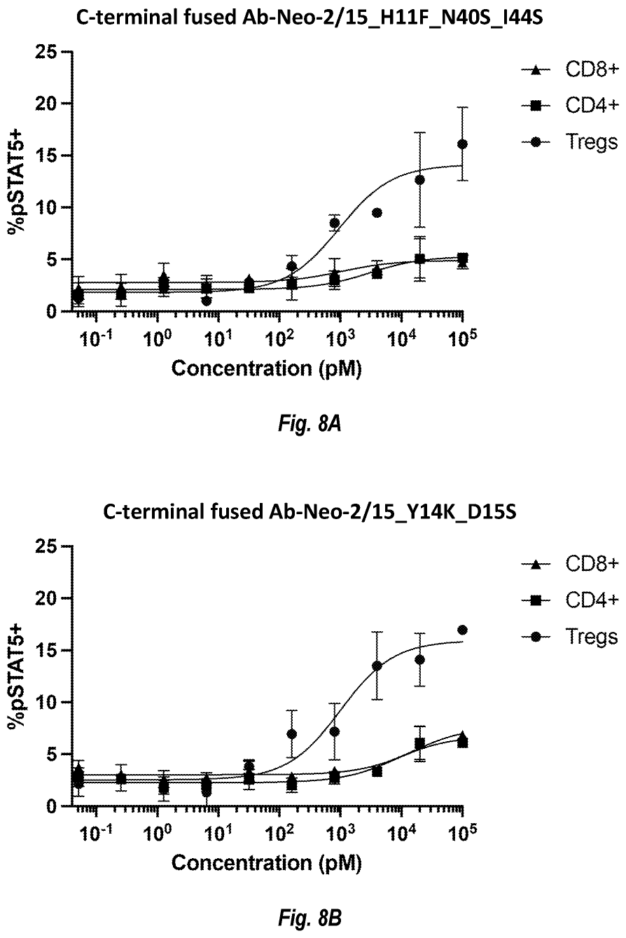

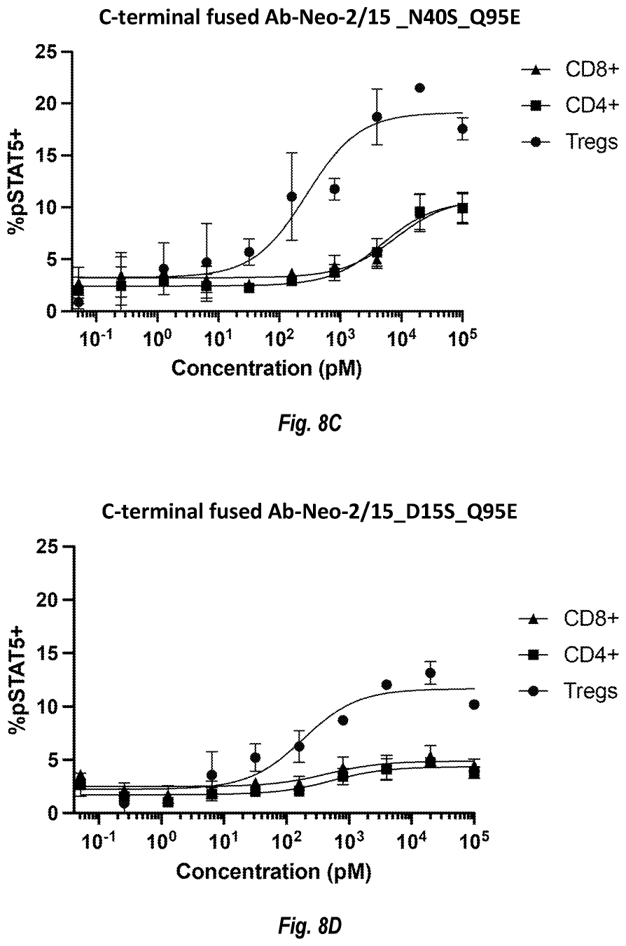

The top C-terminus fusions comprised the following mutations D15S_Q95E; H11F_N40S_I44S; Y14K_D15S; and N40S_Q95E. The fusion protein comprising Neo-2/15_D15S_Q95E demonstrated the best pSTAT5 signaling profile for C-terminus fused TRAs. See FIGS. 7A-D and 8A-D and Table 8 and 9 below. Tables 8 and 9 are values from two different donor samples.

Note that for the fusion protein with the N40S and Q95E mutations at the C terminus, additional mutations of I44S, I44S+H11F, I44S+8 HR, I44S+Y14K had the effect of reducing the Treg STAT5 phosphorylation activity to an undesirable level, if fused at the C terminus.

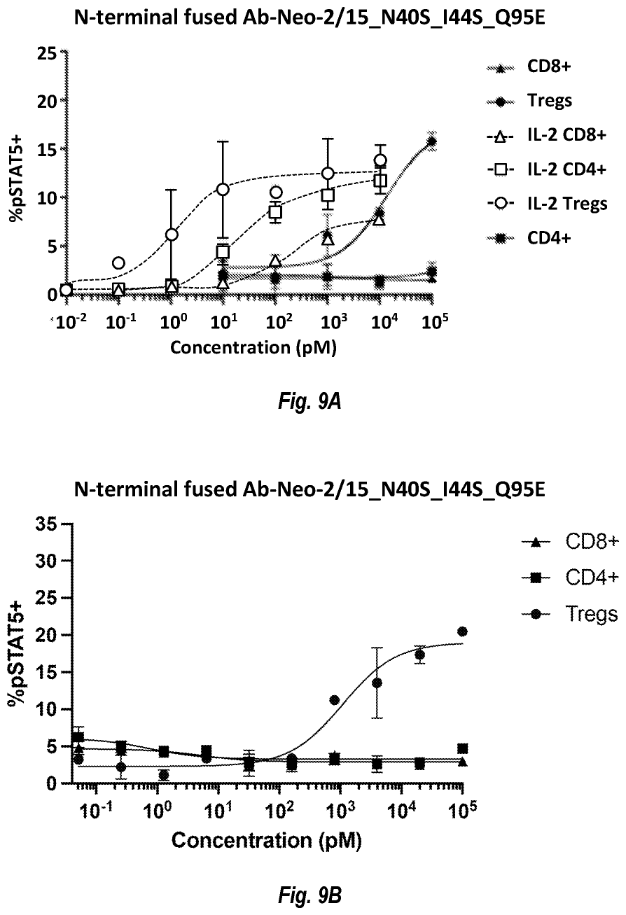

When fused to the N terminus, the top ten fusion proteins comprised the following mutations (i) D15S_N40S; (ii) D15S_N40S_I44S; (iii) H8R_D15S; (iv) H11F_D15S; (v) H11F_N40S_I44S; (vi) H11F_D15S_Q95E; (vii) H11F_N40S_I44S_Q95E; (viii) N40S_I44S_Q95E; (ix) Y14K_D15S_Q95E; and (x) Y14K_N40S_I44S_Q95E. See FIGS. 9A-B and Table 8 and 9 below. Tables 8 and 9 are values from two different donor samples.

TABLE 8

| | CD8+ | | CD4+ | | Treg | |
|---|---|---|---|---|---|---|
| | Ec50 in pM (% Emax) | | | | | |
| C-terminal Fusion | | | | | | |
| Proteins** | | | | | | |
| H11F_N40S_I44S | 91.8 | (1.5%) | 28779 | (2.2%) | 10024 | (8.2%) |
| Y14K_D15S | 3892 | (4%) | 10950 | (6%) | 881 | (13%) |
| N40S_Q95E | 16200 | (4.2%) | 5165 | (5.8%) | 134.6 | (12.2%) |
| D15S_Q95E | 5575 | (0.6%) | 18191 | (0.75) | 1685 | (5%) |
| N-terminal Fusions | | | | | | |
| D15S_N40S | 0.01072 | (3.4%) | 2409 | (5.6%) | 381 | (15.3%) |
| D15S_N40S_I44S | 1066 | (2.8%) | 9065 | (4.5%) | 1418 | (16.7%) |
| H8R_D15S | 155466 | (4.3%) | — | (—) | 13949 | (23.6%) |
| H11F_D15S | 167 | (2.1%) | 1992 | (3.4%) | 332.6 | (12%) |
| H11F-N40S_I44S | — | | 2677 | (3.8%) | 791 | (12.2%) |
| H11F_D15S_Q95E | 4446 | (3.2%) | 7080 | (5.1%) | 7252 | (17.3%) |
| H11F_N40S_I44S_Q95E | 686.3 | (3.1%) | 1303 | (5.1%) | 425 | (18.5%) |
| N40S_I44S_Q95E | 1965 | (1.5%) | — | | 13532 | (17.36%) |
| Y14K_D15S_Q95E | 10203 | (2.9%) | 10674 | (6.2%) | 1262 | (12.7%) |
| Y14K_N40S_I44S_Q95E | 4988 | (2.2%) | 15203 | (2.7%) | 4063 | (11.2%) |
| D15S-Q95E | 5485 | (5.2%) | 953.0 | (8.1%) | 1593 | (13.7%) |

**This donor showed low response compared to previous donors.

TABLE 9

| | CD8+ | | CD4+ | | Treg | |
|---|---|---|---|---|---|---|
| | Ec50 in pM (% Emax) | | | | | |
| C-terminal Fusion | | | | | | |
| Proteins | | | | | | |
| H11F_N40S_I44S | 1030 | (4.9%) | 3019 | (5.3%) | 957.9 | (14.1%) |
| Y14K_D15S | 15849 | (7.6%) | 7833 | (6.7%) | 1006 | (15.9%) |
| N40S_Q95E | 7976 | (10.8%) | 4987 | (10.6%) | 278.1 | (19.15) |
| D15S_Q95E | 446.3 | (4.8%) | 527.2 | (4.4%) | 185.6 | (11.7%) |
| N-terminal Fusion | | | | | | |
| Proteins | | | | | | |
| D15S_N40S | 15.7 | (2.5%) | 26303 | (7%) | 512.8 | (18.25) |
| D15S_N40S_I44S | 20076 | (3.6%) | 20191 | (8%) | 289.1 | (18.4%) |
| H11F_D15S | 5.637 | (2.5%) | 0.6369 | (3.1%) | 4640 | (26%) |
| H11F-N40S_I44S | 5373 | (3.1%) | 7117 | (6.1%) | 529.9 | (17.7%) |
| H11F_N40S_I44S_Q95E | 0.1052 | (4.1%) | 11487 | (9.2%) | 872.2 | (25%) |
| N40S_I44S_Q95E | 5.059 | (2.9%) | 0.7485 | (3.35) | 1093 | (19%) |
| D15S-Q95E | 26377 | (10.5%) | 1335 | (13.8%) | 4.429 | (22.1%) |
| IL-2 | 150 | (10.82%) | 11.49 | (13.79%) | 96.64 | (15%) |

C-terminal fusion with the Neo-2/15_D15S_Q95E mutant showed consistently strong preferential Treg pSTAT5 signaling profile across donors, with low levels of Teff signaling whereas N-terminal fusions showed less consistent profiles across donors, with the exception of protein fusions with the Neo-2/15_Q95E_N40S_I44S mutant. See FIGS. 9A-B.

### Example 9—Treg Expansion in Mice

Fusion proteins comprising Neo-2/15_D15S_Q95E fused to a full length anti-CD25 antibody at either the C and N termini were tested for their ability to expand Treg cells and NK cells in vivo.

Humanized NSG mice were purchased from Jackson Labs. These mice were engrafted with human CD34+ hematopoietic stem cells and have >25% human CD45+ cells in peripheral blood. At Days −1 and 13, mice were injected intraperitoneally with 5 mg of human IgG. On Days 0, 7, 14, and 21, mice were injected intraperitoneally with 15-45 ug of test articles. On Days −1, 6, 13, and 20, 100 μL of whole blood was collected by retro-orbital bleed in K2EDTA. Red blood cells were lysed using BioLegend RBC Lysis Buffer and remaining PBMCs were washed with PBS and then stained with viability stain and human Fc blocking antibodies. Cells were then fixed using eBioscience Foxp3/Transcription Factor Staining Buffer Set. Cells were washed with FACS buffer and then stained using CD3, CD4, CD8, CD25, CD56, and Foxp3 antibodies. Flow cytometry analysis of cells was performed on the Cytek Aurora and analyzed using OMIQ cytometry analysis platform.

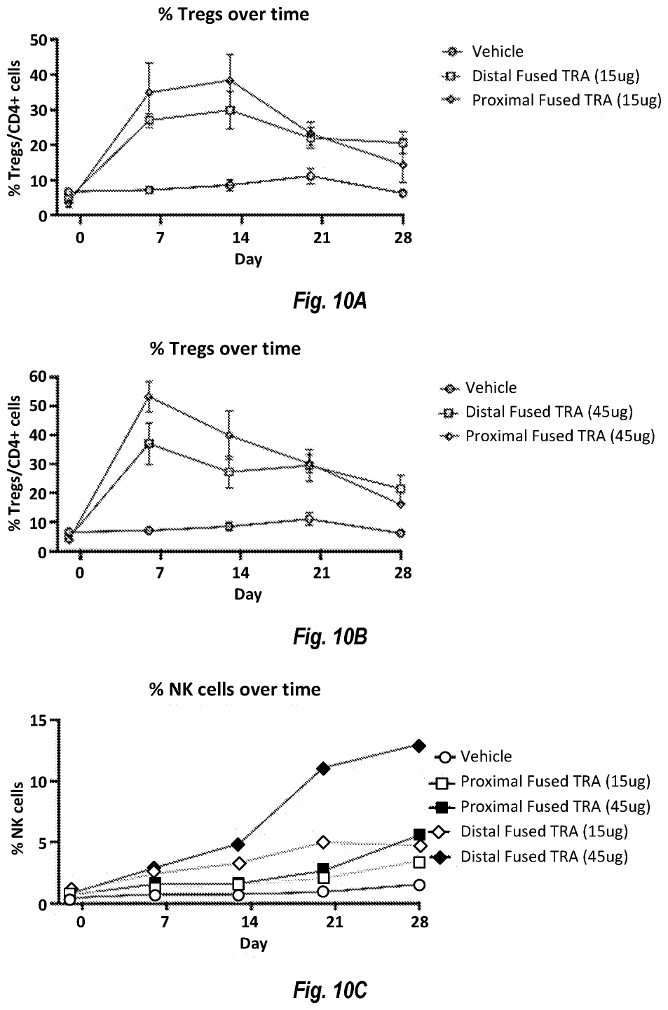

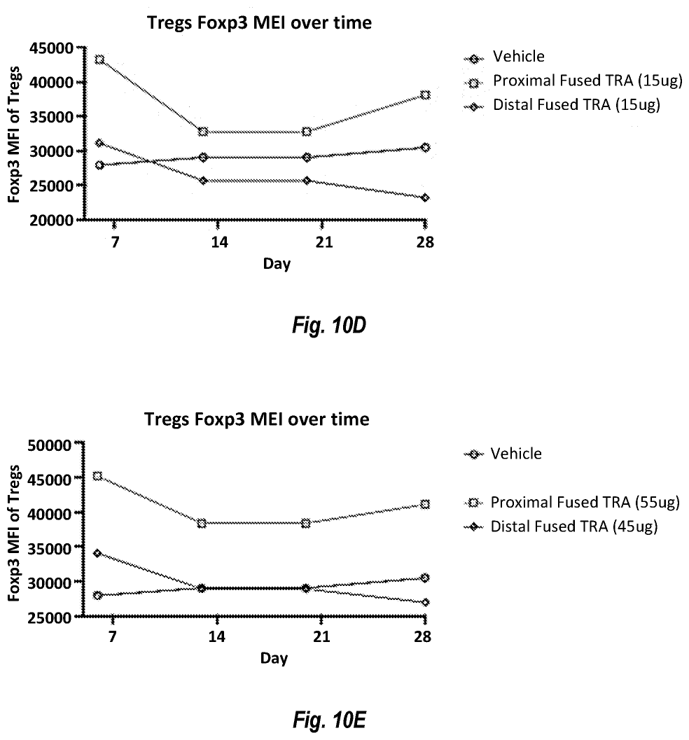

Treg expansion was determined by gating viable cells for CD3 expression, followed by gating for CD4 expression. CD4+ cells were then gated for Tregs by CD25+Foxp3+ stain. Potent Treg expansion was defined as at least 2× higher percentages of Tregs in CD4+ cells when compared to control mice and the Day-1 timepoint. Treg CD25 and Foxp3 MFIs were also compared between groups, with higher MFIs corresponding with a more stable and potent Treg phenotype. NK cells were defined as CD56+ cells when gated on viable cells. Increases in % CD56+ cells were considered an undesirable effect of potential Treg agonists. High Treg expansion was seen with both fusion proteins comprising a full length antibody, however, the N-terminal fusion showed significant NK cell expansion at high dose. See FIGS. 10A-E

An anti-drug antibody (ADA) analysis was performed to determine if the TRAs raise anti-drug antibodies. For this experiment, A ScFv fusion protein with Neo-2/15_D15S_Q95E was used with no Fc domain. No anti-drug antibodies were detected. (Data not shown).

### Example 10—Alternative Treg Targeting Agents

Neo-2/15_D15S_Q95E was fused to an anti-CD39 antibody to test whether a fusion protein targeting to a T-reg marker other than CD25 would also result in a PSTAT5 profile characterized by low Teff signaling with preferential Treg signaling. Although the Treg signal wasn't as high as for the CD25 fusions, preferential Treg signaling was observed. (Data not shown).

### Example 11—IL-2/IL-15 Antagonists are not TRAS

Protein P5 from International Application No. WO2021/188374 was fused to anti-CD25 ScFV and tested for pSTAT5 Treg signaling activity. There was no pSTAT5 signaling for all T cell types tested, CD8+ cells, CD4+ cells and Tregs. (Data not shown).

### Example 12—Generation of Additional TRAs

Another panel of mutations were made in order to identify additional substitutions at positions 95, 40 and 44 that could result in active Treg agonists. Mutants were identified that demonstrate a Treg selective pSTAT5 profile, including Neo-2/15_D15S_Q95K; Neo-2/15_D15S_Q95T; Neo-2/15_D15S_Q95Y; Neo-2/15_N40G_I44S_Q95E; Neo-2/15_N40S_I44T_Q95E; Neo-2/15_N40S_I44Y_Q95E; and Neo-2/15_N40S_I44N_Q95E.

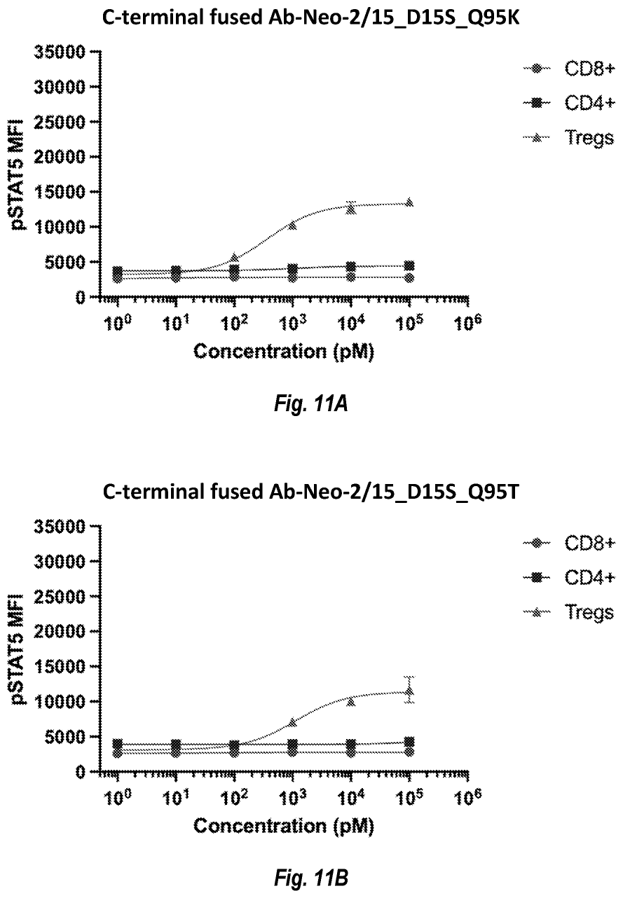

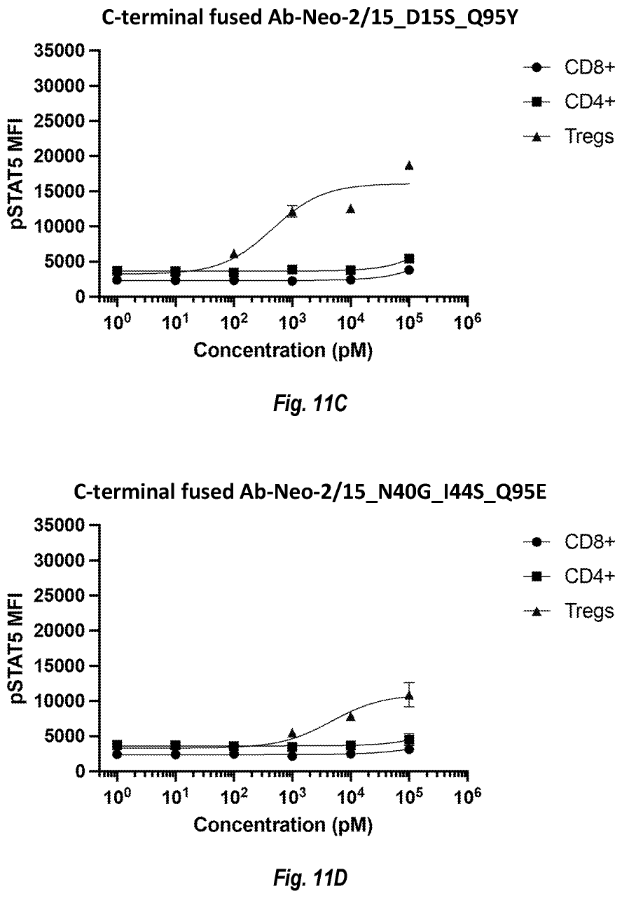

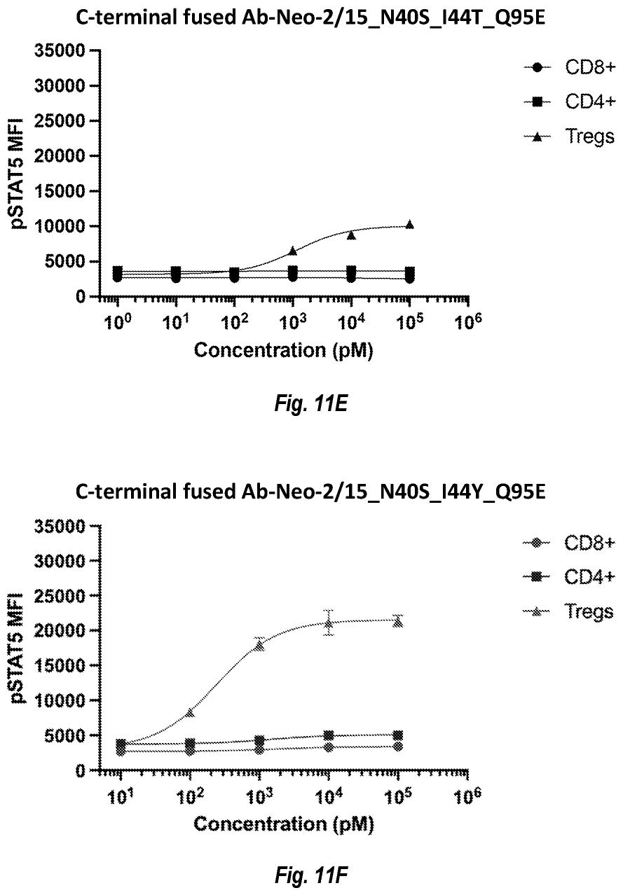

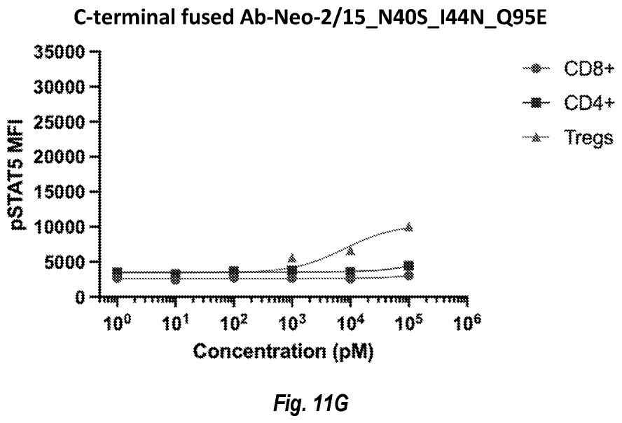

Fusion proteins were made with each mutant expressed as C-terminal fusions to the full length anti-CD25 antibody as previously described. pSTAT5 signaling was plotted using the pSTAT5 mean fluorescence index of each cell subset. See FIGS. 11A-G.

### Example 13—TRAs of the Present Invention Demonstrate Attenuated Binding to IL-2RBG in Cell Binding Assays

Saturation binding studies were done using a HEK293 cell line expressing human CD122 and CD132. Cells were collected with Versene (Gibco, cat #15040-066), washed twice in PBS (Gibco, cat #10010-23), and blocked in 2% BSA (Sigma-Aldrich, cat #A2153-100G) in PBS on ice for 20 minutes. Following blocking, the cells were aliquoted at 2×105 cells per well into a 96-well U-bottom plate (Corning, cat #3799). Antibody fusions were added to the cells at concentrations ranging from 0.5 mM to ˜150 pM or 0.05 mM to ˜15 pM in 1% BSA/PBS. Cells were incubated for 30 minutes on ice, then pelleted and washed twice with cold PBS. The cells were pelleted and resuspended in a 200 ng/ml solution of an APC-conjugated mouse anti-human IgG Fc secondary antibody (Jackson ImmunoResearch, cat #709-605-149) prediluted into 2% BSA/PBS. After a 30-minute incubation on ice, the cells were pelleted and washed twice in cold PBS. Upon resuspension in a 2% paraformaldehyde solution (Acros, cat #41073-0010) to 1×106 cells/mL, fluorescence was detected using a CytoFLEX flow cytometer (Beckman-Coulter). The apparent dissociation constant (KD) was calculated using GraphPad Prism (GraphPad, San Diego, CA).

TABLE 10

| Test Article | Kd (μM) |
|---|---|
| Anti-CD25_Neo-2/15 control | 0.002 |
| C terminal anti-CD25 full-length Ab fusion | 0.131 |
| with Neo-2/15_D15S_Q95E | |
| (TRA comprising SEQ ID NO: 69 and 70) | |
| N terminal anti-CD25 full-length Ab fusion | 0.082 |
| with Neo-2/15_D15S_Q95E | |
| N terminal anti-CD25 full-length Ab fusion | 0.131 |
| with Neo-2/15_N40S_I44S_Q95E | |
| (TRA comprising SEQ ID NO: 74 and 70) | |

TABLE of
Exemplary Sequences

| SEQ | | |
|---|---|---|
| ID | | |
| NO | Description | Sequence |
| 1 | Neo-2/15 (without 3 | KIQLHAEHALYDALMILNIVKTNSPPAEEKLEDYAFNFELILEEIAR |
| | N-terminal amino | LFESGDQKDEAEKAKRMKEWMKRIKTTASEDEQEEMANAIITILQSW |
| | acids; “−3 AA”) | IFS |
| 2 | Neo-2/15 | PKKKIQLHAEHALYDALMILNIVKTNSPPAEEKLEDYAFNFELILEE |
| | | IARLFESGDQKDEAEKAKRMKEWMKRIKTTASEDEQEEMANAIITIL |
| | | QSWIFS |
| 3 | Neo_2/15_ | KIQLHAEHALYSALMILNIVKTNSPPAEEKLEDYAFNFELILEEIAR |
| | D15S_Q95E (−3 AA) | LFESGDQKDEAEKAKRMKEWMKRIKTTASEDEQEEMANAIITILESW |
| | | IFS |
| 4 | Neo_2/15_D15S_ | PKKKIQLHAEHALYSALMILNIVKTNSPPAEEKLEDYAFNFELILEE |
| | Q95E | IARLFESGDQKDEAEKAKRMKEWMKRIKTTASEDEQEEMANAIITIL |
| | | ESWIES |
| 5 | Neo_2/15_D15S_ | PKKKIQLHAEFALYSALMILNIVKTNSPPAEEKLEDYAFNFELILEE |
| | Q95E_H11F | IARLFESGDQKDEAEKAKRMKEWMKRIKTTASEDEQEEMANAIITIL |
| | | ESWIES |
| 6 | Neo_2/15_Q95E_ | KIQLHAEHALYDALMILNIVKTNSPPAEEKLEDYAFSFELSLEEIAR |
| | N40S_144S (−3AA) | LFESGDQKDEAEKAKRMKEWMKRIKTTASEDEQEEMANAIITILESW |
| | | IFS |
| 7 | Neo_2/15_Q95E_ | PKKKIQLHAEHALYDALMILNIVKTNSPPAEEKLEDYAFSFELSLEE |
| | N40S_I44S | IARLFESGDQKDEAEKAKRMKEWMKRIKTTASEDEQEEMANAIITIL |
| | | ESWIFS |
| 8 | Neo_2/15_Q95E_ | PKKKIQLHAEHALYDALMILNIVKTNSPPAEEKLEDYAFAFELALEE |
| | N40A_144A | IARLFESGDQKDEAEKAKRMKEWMKRIKTTASEDEQEEMANAIITIL |
| | | ESWIFS |
| 9 | Neo_2/15_Q95E_ | PKKKIQLHAEHALYDALMILNIVKTNSPPAEEKLEDYAFSFELALEE |
| | N40S_I44A | IARLFESGDQKDEAEKAKRMKEWMKRIKTTASEDEQEEMANAIITIL |
| | | ESWIFS |
| 10 | Neo_2/15_Q95E_ | PKKKIQLHAEHALYDALMILNIVKTNSPPAEEKLEDYAFAFELSLEE |
| | N40A_144S | IARLFESGDQKDEAEKAKRMKEWMKRIKTTASEDEQEEMANAIITIL |
| | | ESWIES |
| 11 | Neo_2/15_ | PKKKIQLHAEFALYDALMILNIVKTNSPPAEEKLEDYAFSFELSLEE |
| | Q95E_N40S_I44S_ | IARLFESGDQKDEAEKAKRMKEWMKRIKTTASEDEQEEMANAIITIL |
| | H11F | ESWIFS |
| 12 | Neo_2/15_ | PKKKIQLHAEHALKDALMILNIVKTNSPPAEEKLEDYAFSFELSLEE |
| | Q95E_N40S_144S_ | IARLFESGDQKDEAEKAKRMKEWMKRIKTTASEDEQEEMANAIITIL |
| | Y14K | ESWIES |
| 13 | Neo_2/15_D15S_ | PKKKIQLHAEHALKSALMILNIVKINSPPAEEKLEDYAFNFELILEE |
| | Q95E_Y14K | IARLFESGDQKDEAEKAKRMKEWMKRIKTTASEDEQEEMANAIITIL |
| | | ESWIFS |
| 14 | Neo_2/15_Q95H_ | KIQLHAEHALYAALMILNIVKTNSPPAEEKLEDYAFNFELILEEIAR |
| | D15A (−3 AA) | LFESGDQKDEAEKAKRMKEWMKRIKTTASEDEQEEMANAIITILHSW |
| | | IFS |
| 15 | Neo_2/15_Q95H_ | PKKKIQLHAEHALYAALMILNIVKINSPPAEEKLEDYAFNFELILEE |
| | D15A | IARLFESGDQKDEAEKAKRMKEWMKRIKTTASEDEQEEMANAIITIL |
| | | HSWIFS |
| 16 | Neo_2/15_D15A_ | KIQLHAEHALYAALMILNIVKTNSPPAEEKLEDYAFNFELILEEIAR |
| | Q95A (−3 AA) | LFESGDQKDEAEKAKRMKEWMKRIKTTASEDEQEEMANAIITILASW |
| | | IFS |
| 17 | Neo_2/15_D15A_ | PKKKIQLHAEHALYAALMILNIVKTNSPPAEEKLEDYAFNFELILEE |
| | Q95A | IARLFESGDQKDEAEKAKRMKEWMKRIKTTASEDEQEEMANAIITIL |
| | | ASWIFS |
| 18 | Neo_2/15_D15S_ | PKKKIQLHAEHALKSALMILNIVKTNSPPAEEKLEDYAFNFELILEE |
| | Y14K | IARLFESGDQKDEAEKAKRMKEWMKRIKTTASEDEQEEMANAIITIL |
| | | QSWIFS |
| 19 | Neo_2/15_ | PKKKIQLHAEFALYDALMILNIVKTNSPPAEEKLEDYAFSFELSLEE |
| | N40S_I44S_H11F | IARLFESGDQKDEAEKAKRMKEWMKRIKTTASEDEQEEMANAIITIL |
| | | QSWIFS |
| 20 | Neo_2/15_ | KIQLHAEFALYDALMILNIVKTNSPPAEEKLEDYAFSFELSLEEIAR |
| | N40S_I44S_H11F | LFESGDQKDEAEKAKRMKEWMKRIKTTASEDEQEEMANAIITILQSW |
| | (−3 AA) | IFS |
| 21 | Neo_2/15_ | PKKKIQLHAEHALYSALMILNIVKTNSPPAEEKLEDYAFSFELSLEE |
| | N40S_I44S_D15S | IARLFESGDQKDEAEKAKRMKEWMKRIKTTASEDEQEEMANAIITIL |
| | | QSWIES |
| 22 | Neo_2/15_ | PKKKIQLHAEHALKSALMILNIVKTNSPPAEEKLEDYAFSFELSLEE |
| | N40S_I44S_Y14K | IARLFESGDQKDEAEKAKRMKEWMKRIKTTASEDEQEEMANAIITIL |
| | D15S | 2SWIFS |
| 23 | Neo_2/15_ | PKKKIQLHAEHALYSALMILNIVKTNSPPAEEKLEDYAFSFELILEE |
| | N40S_D15S | IARLFESGDQKDEAEKAKRMKEWMKRIKTTASEDEQEEMANAIITIL |
| | | QSWIFS |
| 24 | Neo_2/15_ | PKKKIQLRAEHALYSALMILNIVKTNSPPAEEKLEDYAFNFELILEE |
| | H8R_D15S | IARLFESGDQKDEAEKAKRMKEWMKRIKTTASEDEQEEMANAIITIL |
| | | QSWIFS |
| 25 | Neo_2/15_ | PKKKIQLHAEFALYSALMILNIVKINSPPAEEKLEDYAFNFELILEE |
| | H11F_D15S | IARLFESGDQKDEAEKAKRMKEWMKRIKTTASEDEQEEMANAIITIL |
| | | QSWIFS |
| 26 | Daclizumab heavy | QVQLVQSGAEVKKPGSSVKVSCKASGYTFTSYRMHWVRQAPGQGLEW |
| | chain variable region | IGYINPSTGYTEY |
| | (VH) | NQKFKDKATITADESTNTAYMELSSLRSEDTAVYYCARGGGVEDYWG |
| | | QGTLVTVSS |
| 27 | Daclizumab CDR- | SYRMH |
| | H1 | |
| 28 | Daclizumab CDR- | YINPSTGYTEYNQKFKD |
| | H2 | |
| 29 | Daclizumab CDR- | GGGVFDY |
| | H3 | |
| 30 | Daclizumab CDR- | SASSSISYMH |
| | L1 | |
| 31 | Daclizumab CDR- | TTSNLAS |
| | L2 | |
| 32 | Daclizumab CDR- | HQRSTYPLT |
| | L3 | |
| 33 | Daclizumab FR-H1 | QVQLVQSGAEVKKPGSSVKVSCKASGYTFT |
| 34 | Daclizumab FR-H2 | WVRQAPGQGLEWIG |
| 35 | Daclizumab FR-H3 | YINPSTGYTEYNQKEKD |
| 36 | Daclizumab FR-H4 | WGQGTLVTVSS |
| 37 | Daclizumab FR-L1 | DIQMTQSPSTLSAVGDRVTITC |
| 38 | Daclizumab FR-L2 | WYQQKPGKAPKLLIY |
| 39 | Daclizumab FR-L3 | GVPARFSGSGSGTEFTLTISSLQPDDFATYYC |
| 40 | Daclizumab FR-L4 | FGQGTKVEVK |
| 41 | Daclizumab light | DIQMTQSPSTLSASVGDRVTITCSASSSISYMHWYQQKPGKAPKLLI |
| | chain variable region | YTTSNLASGVPAR |
| | (VL) | FSGSGSGTEFTLTISSLQPDDFATYYCHQRSTYPLTFGQGTKVEVK |
| 42 | Daclizumab CDR- | GYRMH |
| | H1 variant 1 | |
| 43 | Daclizumab CDR- | SYRMH |
| | H1 variant 2 | |
| 44 | Daclizumab CDR- | WINPNSGGTNYAQKFQG |
| | H2 variant | |
| 45 | Daclizumab CDR- | RASQSVSYLH |
| | L1 variant | |
| 46 | Daclizumab CDR- | TTSNRAT |
| | L2 variant | |
| 47 | HUMAX-TAC- | QVQLVQSGAEVKKPGSSVKVSCKASGGTFSRYIINWVRQAPGQGLEW |
| | heavy chain variable | MGRIIPILGVENY |
| | region (VH) | AQKFQGRVTITADKSTSTAYMELSSLRSEDTAVYYCARKDWFDYWGQ |
| | | GTLVTVSSASTKG |
| | | PSVFPLA |
| 48 | HUMAX-TAC- | EIVLTQSPGTLSLSPGERATLSCRASQSVSSYLAWYQQKPGQAPRLL |
| | light chain variable | IYGASSRATGIPD |
| | region (VL) | RFSGSGSGTDFTLTISRLEPEDFAVYYCQQYGSSPLTFGGGTKVEIK |
| | | RTVAAPSVFIFP |
| 49 | GS linker | GGGGSGGGGSGGGGSGGGGS |
| 50 | GS linker | GGGGSGGGGSGGGGS |
| 51 | GS linker | GGGGS |
| 52 | GS linker | GGGGGG |
| 53 | GS linker | GGGGGGGG |
| 54 | Rigid linker | EAAK |
| 55 | Rigid linker | EAAKEAAK |
| 56 | Rigid linker | EAAKEAAKEAAK |
| 57 | Rigid linker | EAAAKEAAAKEAAAK |
| 58 | Rigid linker | A(EAAAK)₄ALEA(EAAAK)₄A; |
| 59 | Rigid linker | AEAAAKEAAAKA; |
| 60 | Rigid linker | PAPAP |
| 61 | D1 | KIQLHAEHALYX15ALMILNI |
| | | X15 is serine or alanine |
| 62 | D1 | KIQLHAEHALYDALMILNI |
| 63 | D2 | KDEAEKAKRMKEWMKRIKT |
| 64 | D3 | LEDYAFNFELILEEIARLFESG |
| 65 | D3 | LEDYAFX 40FELX 44 LEEIARL FESG |
| | | X40 is serine or alanine |
| | | x44 is serine or alanine |
| 66 | D4 | EDEQEEMANAIITILX95SWIFS |
| | | X95 is glutamic acid, alanine, or histidine |
| 67 | Linker 1 | VKTNSPPAEE |
| 68 | Linker 3 | TAS |
| 69 | Daclizumab (anti- | QVQLVQSGAEVKKPGSSVKVSCKASGYTFTSYRMHWVRQAPGQGLEW |
| | CD25) VH x huIgG1 | IGYINPSTGYTEYNQKFKDKATITADESTNTAYMELSSLRSEDTAVY |
| | (G1m17 LALA-GA) | YCARGGGVFDYWGQGTLVTVSSASTKGPSVFPLAPSSKSTSGGTAAL |
| | x (G4S)3 #1 x | GCLVKDYFPEPVTVSWNSGALTSGVHTFPAVLQSSGLYSLSSVVTVP |
| | Neo215_D15S_Q95E | SSSLGTQTYICNVNHKPSNTKVDKKVEPKSCDKTHTCPPCPAPEAAG |
| | | APSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFS |
| | | CSVMHEALHNHYTQKSLSLSPGKGGGGSGGGGSGGGGSPKKKIQLHA |
| | | EHALYSALMILNIVKTNSPPAEEKLEDYAFNFELILEEIARLFESGD |
| | | QKDEAEKAKRMKEWMKRIKTTASEDEQEEMANAIITILESWIFS |
| 70 | Daclizumab (anti- | DIQMTQSPSTLSASVGDRVTITCSASSSISYMHWYQQKPGKAPKLLI |
| | CD25) VL x kappa | YTTSNLASGVPARFSGSGSGTEFTLTISSLQPDDFATYYCHQRSTYP |
| | (huIGKC) | LTFGQGTKVEVKRTVAAPSVFIFPPSDEQLKSGTASVVCLLNNFYPR |
| | | EAKVQWKVDNALQSGNSQESVTEQDSKDSTYSLSSTLTLSKADYEKH |
| | | KVYACEVTHQGLSSPVTKSFNRGEC |
| 71 | Human IgG1 | ASTKGPSVFPLAPSSKSTSGGTAALGCLVKDYFPEPVTVSWNSGALT |
| | (LALAGA) | SGVHTFPAVLQSSGLYSLSSVVTVPSSSLGTQTYICNVNHKPSNTKV |
| | | DKKVEPKSCDKTHTCPPCPAPEAAGAPSVFLFPPKPKDTLMISRTPE |
| | | VTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLP |
| | | PSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPG |
| | | K |
| 72 | Human Igκ | RTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGL |
| | | SSPVTKSFNRGEC |
| 73 | Reduced/no effector | QVQLVQSGAEVKKPGSSVKVSCKASGYTFTSYRMHWVRQAPGQGLEW |
| | function Daclizumab | IGYINPSTGYTEYNQKFKDKATITADESTNTAYMELSSLRSEDTAVY |
| | heavy chain (IgG1 | YCARGGGVFDYWGQGTLVTVSSASTKGPSVFPLAPSSKSTSGGTAAL |
| | LALAGA) | GCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAG |
| | | APSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFS |
| | | CSVMHEALHNHYTQKSLSLSPGK |
| 74 | Neo215_N40S_I44S_ | GSPKKKIQLHAEHALYDALMILNIVKTNSPPAEEKLEDYAFSFELSL |
| | Q95E x (G4S)3 #1 | EEIARLFESGDQKDEAEKAKRMKEWMKRIKTTASEDEQEEMANAIIT |
| | x reduced/no | ILESWIFSGGGGSGGGGSGGGGSQVQLVQSGAEVKKPGSSVKVSCKA |
| | effector function | SGYTFTSYRMHWVRQAPGQGLEWIGYINPSTGYTEYNQKEKDKATIT |
| | daclizumab (anti- | ADESTNTAYMELSSLRSEDTAVYYCARGGGVEDYWGQGTLVTVSSAS |
| | CD25) VH x huIgG1 | TKGPSVFPLAPSSKSTSGGTAALGCLVKDYFPEPVTVSWNSGALTSG |
| | (G1m17 LALA-GA) | VHTFPAVLQSSGLYSLSSVVTVPSSSLGTQTYICNVNHKPSNTKVDK |
| | | KVEPKSCDKTHTCPPCPAPEAAGAPSVFLFPPKPKDTLMISRTPEVT |
| | | CVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 75 | Neo_2/15_D15S_Q95K | PKKKIQLHAEHALYSALMILNIVKTNSPPAEEKLEDYAFNFELILEE |
| | | IARLFESGDQKDEAEKAKRMKEWMKRIKTTASEDEQEEMANAIITIL |
| | | KSWIFS |
| 76 | Neo_2/15_D15S_Q95T | PKKKIQLHAEHALYSALMILNIVKTNSPPAEEKLEDYAFNFELILEE |
| | | IARLFESGDQKDEAEKAKRMKEWMKRIKTTASEDEQEEMANAIITIL |
| | | TSWIES |
| 77 | Neo_2/15_D15S_Q95Y | PKKKIQLHAEHALYSALMILNIVKTNSPPAEEKLEDYAFNFELILEE |
| | | IARLFESGDQKDEAEKAKRMKEWMKRIKTTASEDEQEEMANAIITIL |
| | | YSWIFS |
| 78 | Neo_2/15_ | PKKKIQLHAEHALYDALMILNIVKINSPPAEEKLEDYAFGFELSLEE |
| | N40G_I44S_Q95E | IARLFESGDQKDEAEKAKRMKEWMKRIKTTASEDEQEEMANAIITIL |
| | | ESWIES |
| 79 | Neo_2/15_ | PKKKIQLHAEHALYDALMILNIVKTNSPPAEEKLEDYAFSFELTLEE |
| | N40S_I44T_Q95E | IARLFESGDQKDEAEKAKRMKEWMKRIKTTASEDEQEEMANAIITIL |
| | | ESWIES |
| 80 | Neo_2/15_ | PKKKIQLHAEHALYDALMILNIVKINSPPAEEKLEDYAFSFELYLEE |
| | N40S_I44Y_Q95E | IARLFESGDQKDEAEKAKRMKEWMKRIKTTASEDEQEEMANAIITIL |
| | | ESWIES |
| 81 | Neo_2/15_ | KIQLHAEHALYDALMILNIVKINSPPAEEKLEDYAFSFELYLEEIAR |
| | N40S_I44Y_Q95E | LFESGDQKDEAEKAKRMKEWMKRIKTTASEDEQEEMANAIITILESW |
| | (−3AA) | IFS |
| 82 | Neo_2/15_ | PKKKIQLHAEHALYDALMILNIVKTNSPPAEEKLEDYAFSFELNLEE |
| | N40S_I44N_Q95E | IARLFESGDQKDEAEKAKRMKEWMKRIKTTASEDEQEEMANAIITIL |
| | | ESWIES |
| 83 | Rigid linker | APAPAPAPAPAPAPAPAPAPAPAPAPAPAPAPAPAPAPAPAPAPAPA |
| | | PAPAPAPAPAPAPAPAPAPAP |

We claim:

1. A polypeptide comprising a Neo-2/15 mutant, wherein the Neo-2/15 mutant comprises an amino acid sequence at least 80%, at least 85%, or at least 90% identical to SEQ ID NO: 1 or SEQ ID NO: 2, and wherein the Neo-2/15 mutant comprises a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H), and at least one additional substitution selected from:
(a) a serine or alanine in place of aspartic acid at position 15 (D15S or D15A);
(b) an alanine or serine or glycine in place of asparagine at position 40 (N40A or N40S or N40G); and
(c) an alanine or serine or asparagine or threonine or tyrosine in place of isoleucine at position 44 (I44A or I44S or I44N or I44T or I44Y);
wherein the first amino acid of SEQ ID NO:2 is designated position 1, and the first amino acid of SEQ ID NO: 1 is designated position 4.

2. The polypeptide of claim 1, wherein the Neo-2/15 mutant comprises a lysine, a threonine, a tyrosine, a glutamic acid, an alanine, or a histidine in place of glutamine at position 95 (Q95K, Q95T or Q95Y or Q95E or Q95A or Q95H).

3. The polypeptide of claim 1, wherein the at least one additional substituent is selected from:
(a) a serine or alanine in place of aspartic acid at position 15 (D15S or D15A);
(b) an alanine or serine or glycine in place of asparagine at position 40 (N40A or N40S); and
(c) an alanine or serine or asparagine or threonine or serine or tyrosine in place of isoleucine at position 44 (I44A or I44S or I44Y).

4. The polypeptide of claim 1 wherein the Neo-2/15 mutant comprises a glutamic acid, an alanine, or a histidine in place of glutamine at position 95 (Q95E or Q95A or Q95H), and at least one additional substitution selected from:
a) a serine or alanine in place of aspartic acid at position 15 (D15S or D15A);
b) an alanine or serine in place of asparagine at position 40 (N40A or N40S); and
c) an alanine or serine in place of isoleucine at position 44 (I44A or I44S).

5. The polypeptide of any one of claims 1-3, wherein the Neo-2/15 mutant comprises a tyrosine in place of glutamine at position 95 (Q95Y).

6. The polypeptide of any one of claims 1-4, wherein the Neo-2/15 mutant comprises a glutamic acid in place of glutamine at position 95 (Q95E).

7. The polypeptide of any one of claims 1 to 6, wherein the Neo-2/15 mutant comprises a serine or alanine in place of aspartic acid at position 15 (D15S or D15A).

8. The polypeptide of any one of claims 1-7, wherein the Neo-2/15 mutant comprises an alanine in place of aspartic acid at position 15 (D15A).

9. The polypeptide of claim 8, wherein the Neo-2/15 mutant comprises an arginine in place of leucine at position 13.

10. The polypeptide of claim 8 or claim 9, wherein the Neo-2/15 mutant comprises a glutamic acid in place of leucine at position 17.

11. The polypeptide of any one of claims 1-7, wherein the Neo-2/15 mutant comprises a serine in place of aspartic acid at position 15 (D15S).

12. The polypeptide of any one of claims 1-11, wherein the Neo-2/15 mutant comprises a phenylalanine in place of histidine at position 11 (H11F).

13. The polypeptide of any one of claims 1-11, wherein the Neo-2/15 mutant comprises a lysine in place of tyrosine at position 14 (Y14K).

14. The polypeptide of any one of claims 1-12, wherein the Neo-2/15 mutant does not comprise a substitution at any of positions 8, 14, 40, and 44.

15. The polypeptide of claim 14, wherein the Neo-2/15 mutant does not comprise a substitution at any of positions 8, 11, 14, 40, and 44.

16. The polypeptide of any one of claims 1-15, wherein if the Neo-2/15 mutant comprises a serine or alanine in place of aspartic acid at position 15, it does not comprise (i) a serine or alanine or glycine substitution in place of asparagine at position 40 and/or it does not comprise (ii) a serine or alanine or asparagine or threonine or tyrosine substitution in place of isoleucine at position 44.

17. The polypeptide of any one of claims 1-15, wherein if the Neo-2/15 mutant comprises a serine or alanine in place of aspartic acid at position 15, it does not comprise (i) a serine or alanine substitution in place of asparagine at position 40 and/or it does not comprise (ii) a serine or alanine substitution in place of isoleucine at position 44.

18. The polypeptide of any one of claims 1-15, wherein if the Neo-2/15 mutant comprises a serine or alanine in place of aspartic acid at position 15, it does not comprise (i) a substitution at position 40 and/or (ii) a substitution at position 44.

19. The polypeptide of any one of claims 16-18, wherein (i) and (ii) are both true.

20. The polypeptide of any one of claims 1 to 6 wherein the Neo-2/15 mutant comprise an acidic amino acid at position 15.

21. The polypeptide of any one of claims 1 to 6 wherein the Neo-2/15 mutant does not comprise a substitution at position 15.

22. The polypeptide of claim 20 or 21, wherein the Neo-2/15 mutant comprises an alanine or serine or glycine in place of asparagine at position 40 (N40A or N40S or N40G).

23. The polypeptide of claim 22, wherein the Neo-2/15 mutant comprises an alanine or serine in place of asparagine at position 40 (N40A or N40S).

24. The polypeptide of any one of claims 20-23, wherein the Neo-2/15 mutant comprises an alanine or serine or asparagine or threonine or tyrosine in place of isoleucine at position 44 (I44A or I44S or I44N or I44T or I44Y).

25. The polypeptide of claim 24, wherein the Neo-2/15 mutant comprises an alanine or serine in place of isoleucine at position 44 (I44A or I44S).

26. The polypeptide of claim 24, wherein the Neo-2/15 mutant comprises a tyrosine in place of isoleucine at position 44 (I44Y).

27. The polypeptide of any one of claims 20-26, wherein the Neo-2/15 mutant comprises a phenylalanine in place of histidine at position 11 (H11F).

28. The polypeptide of any one of claims 20-27, wherein the Neo-2/15 mutant comprises a lysine in place of tyrosine at position 11 (Y14K).

29. The polypeptide of any one of claims 1-28, wherein the Neo-2/15 mutant does not comprise a substitution at position 8 (8 HR)

30. The polypeptide of any one of claims 1-29, wherein the Neo-2/15 mutant does not comprise a substitution at one or both of positions 17 and 99.

31. The polypeptide of any one of the preceding claims, wherein the Neo-2/15 mutant comprises a set of substitutions selected from:
a) Q95E and D15S;
b) Q95E, D15S, and H11F;
c) Q95E, N40S, and I44S;
d) Q95E, N40S, I44S, and H11F;
e) Q95E, N40S, I44S, and Y14K;
f) Q95E, D15S, and Y14K;
g) Q95H, D15A, L17E, and L13R;
h) Q95H and D15A; and
i) Q95A and D15A.

32. The polypeptide of any one of claims 1 to 30, wherein the Neo-2/15 mutant comprises a set of substitutions selected from:
(a) D15S and Q95K;
(b) D15S and Q95T;
(c) D15S and Q95Y;
(d) N40G, I44S, and Q95E;
(e) N40S, I44T, and Q95E;
(f) N40S, I44Y, and Q95E; and
(g) N40S, I44N, and Q95E.

33. The polypeptide of claim 31, wherein the Neo-2/15 mutant comprises substitutions Q95E and D15S.

34. The polypeptide of any one of claims 31-33, wherein the Neo-2/15 mutant does not comprise any additional substitutions.

35. A polypeptide comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises a serine or an alanine in place of aspartic acid at position 15 (D15S or D15A), and at least one additional substitution selected from:
(a) a lysine, an arginine, a threonine, a serine, a tyrosine, a glutamic acid, an alanine, or a histidine in place of glutamine at position 95 (Q95K or Q95R or Q95T or Q95S or Q95Y or Q95E or Q95A or Q95H);
(b) an arginine in place of histidine at position 8 (H8R);
(c) a phenylalanine in place of histidine at position 11 (H11F);
(d) a lysine in place of tyrosine at position 14 (Y14K);
(e) an alanine or serine in place of asparagine at position 40 (N40A or N40S); and
(f) an alanine or serine or tyrosine in place of isoleucine at position 44 (I44A or I44S or I44Y),
wherein the first amino acid of SEQ ID NO: 1 is designated position 4 and the first amino acid of SEQ ID NO:2 is designated position 1.

36. The polypeptide of claim 35, wherein the at least one additional substitution is selected from:
(a) a glutamic acid, alanine or histidine in place of glutamine at position 95 (Q95E, Q95A or Q95H);
(b) an arginine in place of histidine at position 8 (H8R);
(c) a phenylalanine in place of histidine at position 11 (H11F);
(d) a lysine in place of tyrosine at position 14 (Y14K);
(e) an alanine or serine in place of asparagine at position 40 (N40A or N40S); and
(f) an alanine or serine in place of isoleucine at position 44 (I44A or I44S).

37. The polypeptide of claim 35 or 36, wherein if the Neo-2/15 mutant comprises a substitution at position 95, it does not comprise a substitution at position 40 and/or 44.

38. The polypeptide of any one of claims 35-37, wherein if the Neo-2/15 mutant comprises a substitution at position 40 or 44, it does not comprise a substitution at position 95, 11, or 8.

39. The polypeptide of any one of claims 35-38, wherein if the Neo-2/15 mutant comprises a substitution at position 40 or 44, it does not comprise a substitution at position 14.

40. The polypeptide of any one of claims 35-39, wherein if the Neo-2/15 mutant comprises a serine or alanine in place of aspartic acid at position 15, it does not comprise a substitution at position 14.

41. The polypeptide of any one of claims 1-40, wherein if the Neo-2/15 mutant comprises a serine or alanine in place of aspartic acid at position 15, it does not comprise a lysine substitution in place of tyrosine at position 14.

42. The polypeptide of any one of claims 1-41, wherein if the Neo-2/15 mutant comprises a substitution at position 95, it does not comprise a substitution at position 8.

43. The polypeptide of any one of claims 1-42, wherein if the Neo-2/15 mutant comprises a glutamic acid in place of glutamine at position 95, it does not comprise an arginine substitution in place of histidine at position 8.

44. A polypeptide comprising a Neo-2/15 mutant, wherein the Neo-2/15 mutant comprises an amino acid sequence at least 80%, at least 85%, or at least 90% identical to SEQ ID NO: 1 or SEQ ID NO: 2, wherein the Neo-2/15 mutant comprises:
(a) a phenylalanine in place of histidine at position 11 (H11F);
(b) an alanine or serine in place of asparagine at position 40 (N40A or N40S); and
(c) an alanine or serine or tyrosine in place of isoleucine at position 44 (I44A or I44S or I44Y),
wherein the first amino acid of SEQ ID NO: 1 is designated position 4 and the first amino acid of SEQ ID NO:2 is designated position 1.

45. The polypeptide of claim 44, wherein the Neo-2/15 mutant comprises:
(a) a phenylalanine in place of histidine at position 11 (H11F);
(b) an alanine or serine in place of asparagine at position 40 (N40A or N40S); and
(c) an alanine or serine in place of isoleucine at position 44 (I44A or I44S).

46. The polypeptide of claim 44 or claim 45, wherein the Neo-2/15 mutant does not comprise a substitution at position 15.

47. The polypeptide of any one of claims 44-46, wherein the Neo-2/15 mutant does not comprise a substitution at position 95.

48. The polypeptide of any one of claims 44-47, wherein the Neo-2/15 mutant does not comprise a substitution at position 14 and/or position 8.

49. The polypeptide of any one of claims 35-38, wherein the Neo-2/15 mutant comprises a set of substitutions selected from:
D15S and Y14K;
N40S, I44S, and H11F;
N40S, I44S, and D15S;
N40S, I44S, Y14K, and D15S;
N40S and D15S;
H8R and D15S; and
H11F and D15S.

50. The polypeptide of claim 49, wherein the Neo-2/15 mutant comprises substitutions N40S, I44S, and D15S.

51. The polypeptide of claim 49 or 50 wherein the Neo-2/15 mutant does not comprise any additional substitutions.

52. The polypeptide of any one of the preceding claims, wherein the Neo-2/15 mutant comprises an amino acid sequence that is at least 90%, at least 91%, at least 92%, at least 93%, at least 94%, at least 95%, at least 96% or at least 97% or 98% identical to SEQ ID NO: 1 or SEQ ID NO:2.

53. The polypeptide of any one of the preceding claims, wherein the Neo-2/15 mutant comprises an amino acid sequence that is at least 90%, at least 91%, at least 92%, at least 93%, at least 94% at least 95%, at least 96% or at least 97% at least 98%, at least 99%, or 100% identical to an amino acid sequence selected from SEQ ID NO:3, 6, 14, 16, or 20.

54. The polypeptide of any one of the preceding claims, wherein the Neo-2/15 mutant comprises an amino acid sequence that is at least 90%, at least 91%, at least 92%, at least 93%, at least 94% at least 95%, at least 96% or at least 97% at least 98%, at least 99%, or 100% identical to an amino acid sequence selected from SEQ ID NOs: 2-25 and SEQ ID Nos: 75-82.

55. The polypeptide of any one of claims 1-41, wherein the Neo-2/15 mutant comprises three amino acids N-terminal and attached to the amino acid at position 4, wherein the three amino acids are proline-lysine-lysine, wherein the position numbering is according to SEQ ID NO: 1.

56. The polypeptide of any one of claims 1-55, wherein the Neo-2/15 mutant does not comprise a substitution at 1, 2, 3, or all 4 of positions 91, 92, 96, and 99.

57. The polypeptide of claim 56, wherein the Neo-2/15 mutant does not comprise substitutions at 1, 2, 3, or all 4 of positions 91, 92, 96, or 99.

58. The polypeptide of any one of claims 1-57, wherein the Neo-2/15 mutant does not comprise a substitution at 1, 2, 3, 4, 5, 6, 7, 8, 9, 10, 11, 12, 13, or 14 of positions 6, 7, 10, 18, 33, 36, 37, 39, 43, 47, 84, 85, 88, or 98.

59. The polypeptide of any one of claims 1-58, wherein the Neo-2/15 mutant does not comprise a substitution at positions 91 or 92.

60. The polypeptide of any one of claims 1-59, wherein the Neo-2/15 mutant does not comprise a substitution at positions 8, 11, or 14.

61. The polypeptide of any one of claims 1-60, wherein the Neo-2/15 mutant does not comprise a substitution at positions 13 or 17.

62. The polypeptide of any one of claims 1-61, wherein to the extent the Neo-2/15 mutant has a substitution at a position other than positions 8, 11, 14, 15, 40, 44, or 95, it is a substitution that does not substantially interfere with binding of the Neo-2/15 mutant to IL-2Rβγ.

63. A polypeptide comprising a Neo-2/15 mutant, wherein the Neo-2/15 mutant comprises domains D1, D2, D3, and D4; wherein:
(c) D1 comprises the amino acid sequence: KIQLHAEHALYX₁₅ALMILNI (SEQ ID NO: 61) and D3 comprises the amino acid sequence LEDYAFNFELILEEIARLFESG (SEQ ID NO:64); or
(d) D1 comprises the amino acid sequence: KIQLHAEHALYDALMILNI (SEQ ID NO: 62 and D3 comprises the amino acid sequence LEDYAFX₄₀FELX₄₄LEEIARLFESG (SEQ ID NO:65); and
D2 comprises an amino acid sequence at least 8 amino acids in length;
D4 comprises the amino acid sequence EDEQEEMANAIITILX₉₅SWIFS (SEQ ID NO: 66); wherein:
(v) D1, D2, D3 and D4 may be in any order in the Neo-2/15 mutant;
(vi) amino acid linkers may be present between any of the domains (“domain linkers”);
(vii) X₁₅ is serine or alanine; X₉₅ is glutamic acid, lysine, arginine, threonine, serine, tyrosine, alanine, or histidine; X₄₀ is serine or alanine or glycine; and X₄₄ is serine or alanine or asparagine or threonine or tyrosine;
(viii) wherein the Neo-2/15 mutant contains a total of no more than ten, no more than nine, no more than eight, no more than seven, no more than six, no more than five, no more than four, no more than three, no more than two, no more than one, or zero substitutions at amino acid positions not designated as X.

64. The polypeptide of claim 63 wherein X₄₀ is serine or alanine; and X₄₄ is serine or alanine or tyrosine.

65. The polypeptide of claim 63 wherein X₉₅ is glutamic acid, alanine, or histidine; X₄₀ is serine or alanine; and X₄₄ is serine or alanine.

66. The polypeptide of any one of claims 63-65, wherein D1 comprises the amino acid sequence: KIQLHAEHALYX₁₅ALMILNI (SEQ ID NO:61) and D3 comprises the amino acid sequence LEDYAFNFELILEEIARLFESG (SEQ ID NO:64).

67. The polypeptide of any one of claims 63-66, wherein X₁₅ is serine.

68. The polypeptide of any one of claims 63-67, wherein the asparagine at position 7 and the isoleucine at position 11 of SEQ ID NO:64 are not substituted.

69. The polypeptide of any one of claims 63-65, wherein D1 comprises the amino acid sequence: KIQLHAEHALYDALMILNI (SEQ ID NO:62) and D3 comprises the amino acid sequence LEDYAFX₄₀FELX₄₄LEEIARLFESG (SEQ ID NO:65).

70. The polypeptide of claim 69, wherein X₄₀ is serine.

71. The polypeptide of claim 69 or claim 70, wherein X₄₄ is serine.

72. The polypeptide of any one of claims 69-71, wherein the aspartic acid at position 12 of SEQ ID NO:62 is not substituted.

73. The polypeptide of any one of claims 69-72, wherein the tyrosine at position 11 of SEQ ID NO:62 is substituted.

74. The polypeptide of claim 73, wherein the tyrosine at position 11 of SEQ ID NO: 62 is substituted with lysine.

75. The polypeptide of any one of claims 63-74, wherein the histidine at position 8 of SEQ ID NO: 61 or SEQ ID NO: 62 is substituted.

76. The polypeptide of claim 75, wherein the substituent is phenylalanine (H11F).

77. The polypeptide of any one of claims 63-76, wherein the histidine of position 5, the histidine of position 8, and the tyrosine at position 11 of SEQ ID NO: 61 or SEQ ID NO:62 are not substituted.

78. The polypeptide of any one of claims 63-77, wherein X₉₅ is glutamic acid.

79. The polypeptide of any one of claims 63-78, wherein
(ii) D1 comprises the amino acid sequence set forth in SEQ ID NO:61 and D3 comprises the amino acid sequence set forth in SEQ ID NO:64, wherein the glutamine at position 3, the leucine at position 4, the glutamic acid at position 7, the leucine at position 10, and the methionine at position 15 of SEQ ID NO:61; and the aspartic acid at position 3, the tyrosine at position 4, the phenylalanine at position 6, the leucine at position 10, and the glutamic acid at position 14 of SEQ ID NO:64 are not substituted; or
(ii) D1 comprises the amino acid sequence set forth in SEQ ID NO:62 and D3 comprises the amino acid sequence set forth in SEQ ID NO:65 wherein the glutamine of position 3, the leucine at position 4, the glutamic acid at position 7, the leucine at position 10, and the methionine at position 15 of SEQ ID NO:62; and the aspartic acid at position 3, the tyrosine at position 4, the phenylalanine at position 6, the leucine at position 10, and the glutamic acid at position 14 of SEQ ID NO:65 are not substituted.

80. The polypeptide of any one of claims 63-79, wherein the glutamic acid at position 5, the glutamic acid at position 6, the asparagine at position 9, the isoleucine at position 12, the threonine at position 13, the serine at position 17, the isoleucine at 19 and the phenylalanine at position 20 of SEQ ID NO: 66 are not substituted.

81. The polypeptide of any one of claims 63-80, wherein the leucine at position 14 of SEQ ID NO:61 or SEQ ID NO:62 and at the phenylalanine at position 20 of SEQ ID NO:66 are not substituted.

82. The polypeptide of any one of claims 63-81, wherein the leucine at position 14 of SEQ ID NO:61 or SEQ ID NO:62 and the isoleucine at position 12, the threonine at position 13, the serine at position 17, the isoleucine at position 19 and the phenylalanine at position 20 of SEQ ID NO: 66 are not substituted.

83. The polypeptide of any one of claims 63-82, wherein D3 comprises a lysine at the N-terminus and attached to the amino acid at position 1.

84. The polypeptide of any one of claims 63-83, wherein D2 comprises an amino acid sequence having at least 80% identity to KDEAEKAKRMKEWMKRIKT (SEQ ID NO: 63).

85. The polypeptide of claim 84, wherein D2 comprises the amino acid sequence KDEAEKAKRMKEWMKRIKT (SEQ ID NO:63).

86. The polypeptide of any one of claims 63-85 wherein each amino acid linker is, independently, 1-100, 1-90, 1-80, 1-70, 1-60, 1-50, 1-40, 1-30, 1-20, 1-10, or 2-10 amino acids in length.

87. The polypeptide of any one of claims 63-86, wherein the order of the four domains is D1-D3-D2-D4.

88. The polypeptide of any one of claims 63-86, wherein the order of the four domains is D1-D3-D2-D4, D4-D1-D3-D2, D2-D4-D1-D3, or D3-D2-D4-D1.

89. The polypeptide of any one of claims 63-88, wherein the Neo-2/15 mutant comprises an amino acid sequence that is at least 90%, at least 91%, at least 92%, at least 93%, at least 94% at least 95%, at least 96% or at least 97% at least 98%, at least 99%, or 100% identical to an amino acid sequence selected from SEQ ID NO:3, 6, 14, 16, or 20.

90. The polypeptide of any one of claims 63-89, wherein D1 comprises three amino acids N-terminal and attached to the amino acid at position 1 of SEQ ID NO:61 or SEQ ID NO: 62.

91. The polypeptide of claim 90, wherein the three amino acids are proline-lysine-lysine.

92. The polypeptide of any one of claims 1-91, wherein the Neo-2/15 mutant comprises an amino acid sequence that is at least 90%, at least 95%, or 100% identical to an amino acid sequence selected from SEQ ID NOs: 1-25 and SEQ ID Nos: 75-82.

93. The polypeptide of any one of claims 1-92, wherein the Neo-2/15 mutant binds to IL-2Rβγ with an affinity that is at least 10 fold, or at least 100 fold, or at least 500 fold, or at least 1000 fold, or at least 10,000 fold attenuated as compared to Neo-2/15 and/or IL-2.

94. The polypeptide of any one of claims 1-92, wherein the polypeptide binds to IL-2Rβγ with an affinity that is at least 10 fold, or at least 100 fold, or at least 500 fold, or at least 1000 fold, or at least 10,000 fold attenuated as compared to Neo-2/15 and/or IL-2.

95. The polypeptide of claim 93 or claim 94, wherein the affinity is not more than 500 fold attenuated as compared to Neo-2/15 and/or IL-2.

96. The polypeptide of claim 93 or claim 94, wherein the affinity is not more than 1000 fold or not more than 10,000 fold attenuated as compared to Neo-2/15 and/or IL-2

97. The polypeptide of any one of claims 1-96, wherein the Neo-2/15 mutant binds IL-2Rβγ with an affinity that is within 2-fold, 3-fold, 4-fold, or 5-fold of the affinity of a Neo-2/15 mutant consisting of the sequence set forth in SEQ ID NO:4 for IL-2Rβγ.

98. The polypeptide of any one of claims 1-97, wherein the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, binds IL-2Rβγ with an affinity that is within 2-fold, 3-fold, 4-fold or 5-fold of the affinity of a reference polypeptide for IL-2Rβγ, wherein the reference polypeptide comprises a Neo-2/15 mutant fused to the C-terminus of an antibody, wherein the reference polypeptide comprises the amino acid sequences of SEQ ID NOs: 69 and 70.

99. The polypeptide of any one of claims 1-98, wherein the Neo-2/15 mutant stimulates STAT5 phosphorylation in Treg cells with an EC50 that is substantially the same or less than a Neo-2/15 mutant consisting of the sequence set forth in SEQ ID NO:4

100. The polypeptide of any one of claims 1-99, wherein the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with an EC50 that is substantially the same or less than a reference polypeptide comprising a Neo-2/15 mutant fused to the C-terminus of an antibody, wherein the reference polypeptide comprises the amino acid sequences of SEQ ID NOs: 69 and 70.

101. The polypeptide of any one of claims 1-100, wherein the Neo-2/15 mutant stimulates STAT5 phosphorylation in Treg cells with a maximal signaling value that is substantially the same or greater than a Neo-2/15 mutant consisting of the sequence set forth in SEQ ID NO:4.

102. The polypeptide of any one of claims 1-101, wherein the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with a maximal signaling value that is substantially the same or greater than a reference polypeptide comprising a Neo-2/15 mutant fused to the C-terminus of an antibody, wherein the reference polypeptide comprises the amino acid sequences of SEQ ID NOs: 69 and 70.

103. The polypeptide of any one of claims 1-102, wherein the Neo-2/15 mutant stimulates STAT5 phosphorylation in Treg cells with a maximal signaling value that is at least 50%, at least 75%, at least 100% of the maximal signaling resulting from Neo-2/15 and/or IL-2 stimulation.

104. The polypeptide of any one of claims 1-103, wherein the Neo-2/15 mutant, when fused to the N-terminus and/or the C-terminus of an antibody comprising a heavy chain comprising the amino acid sequence of SEQ ID NO: 73 and a light chain comprising the amino acid sequence of SEQ ID NO: 70, stimulates STAT5 phosphorylation in Treg cells with an EC50 of 1 nM or less, 0.5 nM or less, or 0.1 nM or less and/or stimulates STAT5 phosphorylation in Teff cells with an EC50 of 1 nM or more.

105. The polypeptide of any one of claims 1-104, wherein the polypeptide consists of the Neo-2/15 mutant.

106. The polypeptide of any one of claims 1-104 wherein the polypeptide comprises a Fc domain.

107. The polypeptide of any one of claim 1-104 or 106, wherein the polypeptide comprises a targeting agent.

108. The polypeptide of claim 107, wherein the targeting agent binds an antigen on the surface of Treg cells.

109. The polypeptide of claim 108, wherein the antigen is CD25 or CD39.

110. The polypeptide of any one of claims 107-109, wherein the targeting agent is an antibody or antigen-binding fragment thereof.

111. The polypeptide of claim 110, wherein the targeting agent is an antigen-binding fragment of an antibody.

112. The polypeptide of claim 111, wherein the antigen-binding fragment is a Fab, a (Fab′)2, a scFv, or the antigen-binding portion of a single-domain antibody.

113. The polypeptide of claim 112, wherein the antigen-binding fragment is a scFv.

114. The polypeptide of claim 110, wherein the targeting agent is full length antibody.

115. The polypeptide of any one of claims 110-114, wherein the antibody or antigen-binding fragment thereof comprises a CDR-H1 comprising the amino acid sequence of SEQ ID NO: 27, 42, or 43; a CDR-H2 comprising the amino acid sequence of SEQ ID NO: 28 or 44; a CDR-H3 comprising the amino acid sequence of SEQ ID NO: 29; a CDR-L1 comprising the amino acid sequence of SEQ ID NO: 30 or 45; a CDR-L2 comprising the amino acid sequence of SEQ ID NO: 31 or 46; and a CDR-L3 comprising the amino acid sequence of SEQ ID NO: 32.

116. The polypeptide of any one of claims 110-115, wherein the antibody or antigen-binding fragment thereof comprises a heavy chain variable region comprising the amino acid sequence of SEQ ID NO: 26 and a light chain variable region comprising the amino acid sequence of SEQ ID NO: 41.

117. The polypeptide of any one of claims 106-116, wherein the polypeptide comprises an Fc domain that substantially lacks effector function.

118. The polypeptide of claim 117, wherein the Fc domain is an IgG1 Fc domain comprising substitutions L234A and L235A.

119. The polypeptide of claim 118, wherein the Fc domain comprises substitution G237A.

120. The polypeptide of any one of claims 117-119, wherein the Fc domain comprises substitution P329G or P329A.

121. The polypeptide of any one of claims 106-120, wherein the polypeptide comprises a heavy chain IgG1 constant region comprising the amino acid sequence of SEQ ID NO: 71.

122. The polypeptide of any one of claims 106-120, wherein the Neo-2/15 mutant is linked to the N-terminus of the targeting agent or Fc domain.

123. The polypeptide of any one of claims 106-120, wherein the Neo-2/15 mutant is linked to the C-terminus of the targeting agent or Fc domain.

124. The polypeptide of claim 122 or claim 123, wherein the Neo-2/15 mutant is fused to the targeting agent or Fc domain.

125. The polypeptide of any one of claims 122-124 wherein the Neo-2/15 mutant is linked to the targeting agent or Fc domain via an amino acid linker.

126. The polypeptide of claim 125 wherein the amino acid linker comprises the sequence (GGGGS) n wherein n is from 1-10.

127. The polypeptide of claim 126 wherein n is 1, 2, 3 or 4.

128. The polypeptide of claim 123, wherein the polypeptide comprises the amino acid sequence of SEQ ID NO: 69 and the amino acid sequence of SEQ ID NO: 70.

129. The polypeptide of claim 122, wherein the polypeptide comprises the amino acid sequence of SEQ ID NO: 74 and the amino acid sequence of SEQ ID NO: 70.

130. The polypeptide of any one of claims 1-129, wherein the polypeptide does not substantially activate Teff cells, or wherein the polypeptide activates Teff cells with an EC50 that is more than 5-fold, more than 10-fold, more than 50-fold, more than 100-fold, more than 500-fold, or more than 1000-fold higher than the EC50 for activating Treg cells.

131. The polypeptide of any one of claims 1-130, wherein the polypeptide does not substantially induce proliferation of Teff cells, or wherein the polypeptide induces proliferation of Teff cells with an EC50 that is more than 5-fold, more than 10-fold, more than 50-fold, more than 100-fold, more than 500-fold, or more than 1000-fold higher than the EC50 for inducing proliferation of Treg cells.

132. The polypeptide of any one of claims 1-131 wherein the maximal signaling by the polypeptide in non-Treg cells is less than 30% or less than 20% of the maximal signaling of the non-Treg cells by IL-2 at a concentration of 10 nM or less of the polypeptide.

133. The polypeptide of claim 132, wherein the non-Treg cells are Teff cells.

134. The polypeptide of claim 132, wherein the non-Treg cells are NK cells.

135. The polypeptide of any one of claims 1-134, wherein the maximal signaling by the polypeptide in Tregs is at least 50% of the maximal signaling in Tregs by IL-2 at a concentration of 10 nM or less of the polypeptide.

136. The polypeptide of any one of claims 1-135, wherein the polypeptide induces STAT5 phosphorylation in Treg cells with an EC50 below 1 nM, below 500 pM, or below 100 pM and/or the polypeptide induces STAT5 phosphorylation in Teff cells with an EC50 above 1000 pM.

137. The polypeptide of any one of claims 1-136, wherein the polypeptide does not substantially activate NK cells, or wherein the polypeptide activates NK cells with an EC50 that is more than 5-fold, more than 10-fold, more than 50-fold, more than 100-fold, more than 500-fold, or more than 1000-fold higher than the EC50 for activating Treg cells.

138. The polypeptide of any one of claims 1-137 wherein the polypeptide does not substantially induce proliferation of NK cells, or wherein the polypeptide induces proliferation of NK cells with an EC50 that is more than 5-fold, more than 10-fold, more than 50-fold, more than 100-fold, more than 500-fold, or more than 1000-fold higher than the EC50 for inducing proliferation of Treg cells.

139. The polypeptide of any one of claims 1-138, wherein the polypeptide induces STAT5 phosphorylation in Treg cells with an EC50 below 1 nM, below 500 pM, or below 100 pM and/or the polypeptide induces STAT5 phosphorylation in NK cells with an EC50 above 1000 pM.

140. A pharmaceutical composition comprising the polypeptide of any one of claims 1-139 and a pharmaceutically acceptable carrier or diluent.

141. An isolated polynucleotide comprising a polynucleotide sequence that encodes the polypeptide of any one of claims 1-139.

142. A vector comprising the polynucleotide of claim 141.

143. An isolated host cell comprising the polynucleotide of claim 141 or the vector of claim 142.

144. An isolated host cell that expresses the polypeptide of any one of claims 1-139.

145. A method of producing a polypeptide, comprising incubating the host cell of claim 143 or claim 144 under conditions suitable for expressing the polypeptide.

146. The method of claim 145, further comprising isolating the polypeptide.

147. A method of inducing proliferation of Treg cells, comprising contacting the Treg cells with a polypeptide of any one of claims 1-139.

148. A method of activating Treg cells, comprising contacting the Treg cells with a polypeptide of any one of claims 1-139.

149. The method of claim 147 or claim 148, wherein the Treg cells are in vitro or in vivo.

150. A method of treating a disease associated with Teff cell activity and/or B cell activity, comprising administering to a subject in need thereof a polypeptide of any one of claims 1-139 or the pharmaceutical composition of claim 140.

151. The method of claim 150, wherein the subject is suffering from an autoimmune disease.

152. A method of treating an autoimmune disease, comprising administering to a subject in need thereof a polypeptide of any one of claims 1-139 or the pharmaceutical composition of claim 140.

153. The method of claim 151 or claim 152, wherein the autoimmune disease is selected from the group consisting of a rheumatic disease, including, but not limited to, rheumatoid arthritis, systemic lupus erythematosus, Sjogren's syndrome, scleroderma, mixed connective tissue disease, dermatomyositis, polymyositis, Reiter's syndrome or Behcet's disease; type II diabetes; an autoimmune disease of the thyroid, including, but not limited, Hashimoto's thyroiditis or Graves' Disease; an autoimmune disease of the central nervous system, including, but not limited to, multiple sclerosis, myasthenia gravis, or encephalomyelitis; (5) phemphigus, including but not limited to, phemphigus vulgaris, phemphigus vegetans, phemphigus foliaceus, Senear-Usher syndrome, or Brazilian phemphigus; psoriasis; inflammatory bowel disease, including, but not limited to ulcerative colitis or Crohn's Disease; and celiac disease.

154. A method for treating a subject who has received a transplant of biological materials, such as an organ, tissue, or cell transplant, comprising administering to the subject the polypeptide of any one of claims 1-139 or the pharmaceutical composition of claim 140.

155. A method of treating Graft vs. Host Disease in a subject, comprising administering to a subject in need thereof a polypeptide of any one of claims 1-139 or the pharmaceutical composition of claim 140.

156. The method of any one of claims 140-155, wherein the subject is human.
